(19) United States
(12) Patent Application Publication
(10) Pub. No.: US 2010/0323990 A1
(43) Pub. Date: Dec. 23, 2010

(54) SHIP 1 MODULATOR PRODRUGS

(75) Inventors: Raymond Andersen, Vancouver (CA); Matthew Nodwell, Vancouver (CA); Alice Mui, Burnaby (CA)

Correspondence Address: SEED INTELLECTUAL PROPERTY LAW GROUP PLLC, 701 FIFTH AVE, SUITE 5400, SEATTLE, WA, 98104, US

(73) Assignee: THE UNIVERSITY OF BRITISH COLUMBIA, Vancouver, BC (CA)

(21) Appl. No.: 12/305,459

(22) PCT Filed: Jun. 21, 2007
(86) PCT No.: PCT/CA2007/001106

§ 371 Date: Mar. 12, 2009

Related U.S. Application Data
(60) Provisional application No. 60/815,258, filed on Jun. 21, 2006.

(51) Int. Cl. A61K 31/66; A61K 31/415; A61K 31/22; C07D 233/00; C07C 381/00; C07C 229/28; C07F 9/02; A61P 1/00; A61P 29/00; A61P 35/00; A61P 35/02; A61P 37/08; A61P 19/02

(52) U.S. Cl. 514/106; 514/143; 514/400; 514/550; 514/551; 548/339.1; 560/147; 560/169; 568/14

(57) ABSTRACT

The present invention provides the use of prodrugs of pelorol and homopelorol, related compounds and pharmaceutical compositions thereof as modulators of SHIP1 activity. A compound or a pharmaceutical composition of the present invention may be used for the treatment or prophylaxis of an inflammatory, neoplastic, hematopoetic or immune disorder or condition in addition to other disorders and conditions.

### TECHNICAL FIELD

The present invention relates to SHIP 1, a negative regulator of cell proliferation and survival and immune cell activation.

### BACKGROUND OF THE INVENTION

SH₂-containing inositol 5-phosphatase (SHIP 1), selectively hydrolyzes the 5-phosphate from inositol 1,3,4,5-tetraphosphate (IP4) and phosphatalidylinositol 3,4,5-triphosphate (PIP3). U.S. Pat. No. 6,238,903 discloses that SHIP 1 is an enzyme regulator of signaling pathways that control gene expression, cell proliferation, differentiation, activation, and metabolism, particularly of the Ras and phospholipid signaling pathways. SHIP 1 plays an important role in cytokine and immune receptor signal tansduction. SHIP 1 disrupted (SHIP 1^{-/-}) mice exhibit a myeloproliferative phenotype characterized by overproduction of granulocytes and macrophages. (Huber, M. et al. (1999) Prog Biophys Mol Biol 71:423) SHIP 1^{-/-} mast cells are more prone to IgE and Steel factor induced degranulation, while SHIP 1^{-/-} B cells are resistant to negative regulation by Fc RIIB. SHIP 1 is also involved in the pathogenesis of chronic myelogenous leukemia. (Sattler, M. et al. (1999) Mol Cell Biol 19:7473)

SHIP 1 is expressed only in blood cells and is an important negative regulator of hemopoietic cell growth/survival and immune cell activation. The specialized function of SHIP 1 has been studied in mouse and man.

Various agonists of SHIP 1 activity are known from WO 2004/035601. An example of an agonist is the sesquiterpene compound pelorol, which was first obtained from marine sponge species. Its synthesis is described in WO 2004/035601. The precise structure of pelorol is as follows, with Me representing a methyl group and relative configuration of chiral atoms (C-5, 8, 9 and 10) shown.

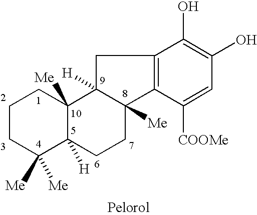

### SUMMARY

This invention is based on the discovery that the efficacy of pelorol and related compounds as modulators of SHIP 1 activity may be improved by adding solubilizing moieties to the compounds.

In illustrative embodiments of the present invention, there is provided a compound of Formula II or a salt thereof:

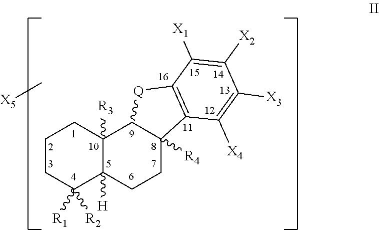

wherein;
R₁ and R₂ are independently selected from the group consisting of: H, —CH₃, —CH₂CH₃, —CH₂OH, —CH₂OR₁′, —CHO, —CO₂H, and —CO₂R₂′;
R₃ and R₄ are independently selected from the group consisting of: H, —CH₃, —CH₂CH₃, —CH₂OH, —CH₂OR₃′, —CHO, —CO₂H, and —CO₂R₄′;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂—, and —CY₁Y₂CY₃Y₄CY₅Y₆—; where Y₁, Y₂, Y₃, Y₄, Y₅, and Y₆ are independently selected from the group consisting of: H, F, Br, Cl, I, OH, OR₅′, SH, any one group of Y₁/Y₂, Y₃/Y₄, and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: H, X₅, R₆′, OH, —O—(C₁-C₁₀ alkyl), —CO₂H, —CO₂R₇′, F, Br, Cl, I, —CN, —SO₃H, —OSO₃H, NO₂, NH₂, —NHR₈′, and —N(R₉′)₂; where R₆′, R₈′ and R₉′ are independently X₅, or a linear, branched, or cyclic, saturated or unsaturated one to ten carbon alkyl group that is unsubstituted or is substituted with one or more of: X₅, OH, ═O, SH, F, Br, Cl, I, NH₂, —NHR₁₀′, —N(R₁₁′)₂, NO₂, —CO₂H, —CO₂R₁₂′, and epoxide;
R₁′, R₂′, R₃′, R₄′, R₅′, R₇′, R₁₀′, R₁₁′, and R₁₂′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group; and
X₅ is a prodrug moiety and at least one of R₁, R₂, R₃, R₄, X₁, X₂, X₃ and X₄ are X₅, comprise X₅ as a substituent or X₅ is a substituent on any carbon atom in Q or in positions 1, 2, 3, 4, 5, 6, 7, 8, 9 and/or 10 of Formula II.

In other illustrative embodiments of the present invention, there is provided a compound of Formula III or a salt thereof:

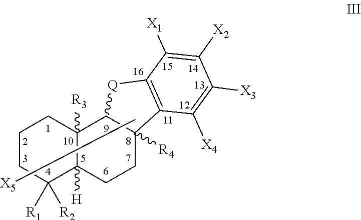

wherein;
R₁ and R₂ are independently selected from the group consisting of: H, —CH₃, —CH₂CH₃, —CH₂OH, —CH₂OR₁′, —CHO, —CO₂H, and —CO₂R₂′;
R₃ and R₄ are independently selected from the group consisting of: H, —CH₃, —CH₂CH₃, —CH₂OH, —CH₂OR₃′, —CHO, —CO₂H, and —CO₂R₄′;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂—, and —CY₁Y₂CY₃Y₄CY₅Y₆—; where Y₁, Y₂, Y₃, Y₄, Y₅, and Y₆ are independently selected from the group consisting of: H, F, Br, Cl, I, OH, OR₅′, SH, any one group of Y₁/Y₂, Y₃/Y₄, and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: H, R₆′, OH, —O—(C₁-C₁₀ alkyl), —CO₂H, —CO₂R₇′, F, Br, Cl, I, —CN, —SO₃H, —OSO₃H, NO₂, NH₂, —NHR₈′, and —N(R₉′)₂; where R₆′, R₈′ and R₉′ are independently a linear, branched, or cyclic, saturated or unsaturated one to ten carbon alkyl group that is unsubstituted or is substituted with one or more of: OH, ═O, SH, F, Br, Cl, I, NH₂, —NHR₁₀′, —N(R₁₁′)₂, NO₂, —CO₂H, —CO₂R₁₂′, and epoxide;
R₁′, R₂′, R₃′, R₄′, R₅′, R₇′, R₁₀′, R₁₁′, and R₁₂′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group; and
X₅ is a prodrug moiety and at least one of R₁, R₂, R₃, R₄, are X₅, comprise X₅ as a substituent or X₅ is a substituent on any carbon atom in Q or in positions 1, 2, 3, 4, 5, 6, 7, 8, 9 and/or 10 of Formula III.

In other illustrative embodiments of the present invention, there is provided a compound of Formula IV or a salt thereof:

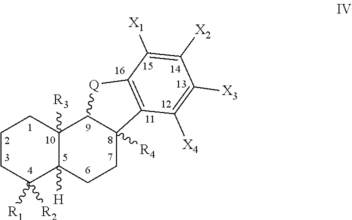

wherein;
R₁ and R₂ are independently selected from the group consisting of: H, —CH₃, —CH₂CH₃, —CH₂OH, —CH₂OR₁′, —CHO, —CO₂H, and —CO₂R₂′;
R₃ and R₄ are independently selected from the group consisting of: H, —CH₃, —CH₂CH₃, —CH₂OH, —CH₂OR₃′, —CHO, —CO₂H, and —CO₂R₄′;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂—, and —CY₁Y₂CY₃Y₄CY₅Y₆—; where Y₁, Y₂, Y₃, Y₄, Y₅, and Y₆ are independently selected from the group consisting of: H, F, Br, Cl, I, OH, OR₅′, SH, any one group of Y₁/Y₂, Y₃/Y₄, and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: H, X₅, R₆′, OH, —O—(C₁-C₁₀ alkyl), —CO₂H, —CO₂R₇′, F, Br, Cl, I, —CN, —SO₃H, —OSO₃H, NO₂, NH₂, —NHR₈′, and —N(R₉′)₂; where R₆′, R₈′ and R₉′ are independently X₅, or a linear, branched, or cyclic, saturated or unsaturated one to ten carbon alkyl group that is unsubstituted or is substituted with one or more of: X₅, OH, ═O, SH, F, Br, Cl, I, NH₂, —NHR₁₀′, —N(R₁₁′)₂, NO₂, —CO₂H, —CO₂R₁₂′, and epoxide;
R₁′, R₂′, R₃′, R₄′, R₅′, R₇′, R₁₀′, R₁₁′, and R₁₂′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group; and
X₅ is a prodrug moiety and at least one of X₁, X₂, X₃ and X₄ are X₅ or comprise X₅ as a substituent.

In other illustrative embodiments of the present invention, there is provided a compound of any formula described herein wherein R₁ and R₂ are independently selected from the group consisting of: —CH₃, —CH₂CH₃, —CH₂OH, —CH₂OR′, —CHO, —CO₂H, and —CO₂R′;

In other illustrative embodiments of the present invention, there is provided a compound of any formula described herein wherein R₁ is selected from the group consisting of: methyl, ethyl, —CH₂OH and —CH₂OR₁′.

In other illustrative embodiments of the present invention, there is provided a compound of any formula described herein wherein R₂ is selected from the group consisting of: methyl, ethyl, —CH₂OH and —CH₂OR₁′.

In other illustrative embodiments of the present invention, there is provided a compound of any formula described herein wherein R₁′ and R₂′ in at least one of R₁ and R₂ is selected from the group consisting of: methyl, ethyl, propyl and butyl.

In other illustrative embodiments of the present invention, there is provided a compound of any formula described herein wherein R₁ is methyl or ethyl.

In other illustrative embodiments of the present invention, there is provided a compound of any formula described herein wherein R₂ is methyl or ethyl.

In other illustrative embodiments of the present invention, there is provided a compound of any formula described herein wherein R₁ is methyl.

In other illustrative embodiments of the present invention, there is provided a compound of any formula described herein wherein R₂ is methyl.

In other illustrative embodiments of the present invention, there is provided a compound of Formula V or a salt thereof:

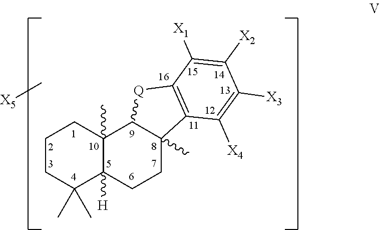

wherein;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂—, and —CY₁Y₂CY₃Y₄CY₅Y₆—; where Y₁, Y₂, Y₃, Y₄, Y₅, and Y₆ are independently selected from the group consisting of: H, F, Br, Cl, I, OH, OR₅′, SH, any one group of Y₁/Y₂, Y₃/Y₄, and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: H, X₅, R₆′, OH, —O—(C₁-C₁₀ alkyl), —CO₂H, —CO₂R₇′, F, Br, Cl, I, —CN, —SO₃H, —OSO₃H, NO₂, NH₂, —NHR₈′, and —N(R₉′)₂; where R₆′, R₈′ and R₉′ are independently X₅, or a linear, branched, or cyclic, saturated or unsaturated one to ten carbon alkyl group that is unsubstituted or is substituted with one or more of: X₅, OH, ═O, SH, F, Br, Cl, I, NH₂, —NHR₁₀′, —N(R₁₁′)₂, NO₂, —CO₂H, —CO₂R₁₂′, and epoxide;
R₅′, R₇′, R₁₀′, R₁₁′, and R₁₂′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group; and
X₅ is a prodrug moiety and at least one of X₁, X₂, X₃ and X₄ are X₅, comprise X₅ as a substituent or X₅ is a substituent on any carbon atom in Q or in positions 1, 2, 3, 4, 5, 6, 7, 8, 9 and/or 10 of Formula V.

In other illustrative embodiments of the present invention, there is provided a compound of Formula VI or a salt thereof:

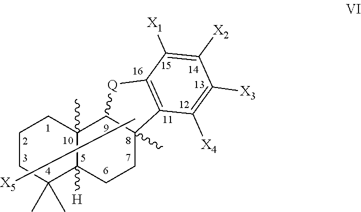

wherein;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂—, and —CY₁Y₂CY₃Y₄CY₅Y₆—; where Y₁, Y₂, Y₃, Y₄, Y₅, and Y₆ are independently selected from the group consisting of: H, F, Br, Cl, I, OH, OR₅′, SH, any one group of Y₁/Y₂, Y₃/Y₄, and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: H, R₆′, OH, —O—(C₁-C₁₀ alkyl), —CO₂H, —CO₂R₇′, F, Br, Cl, I, —CN, —SO₃H, —OSO₃H, NO₂, NH₂, —NHR₈′, and —N(R₉′)₂; where R₆′, R₈′ and R₉′ are independently a linear, branched, or cyclic, saturated or unsaturated one to ten carbon alkyl group that is unsubstituted or is substituted with one or more of: OH, ═O, SH, F, Br, Cl, I, NH₂, —NHR₁₀′, —N(R₁₁′)₂, NO₂, —CO₂H, —CO₂R₁₂′, and epoxide;
R₅′, R₇′, R₁₀′, R₁₁′, and R₁₂′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group; and
X₅ is a prodrug moiety and at least one of R₁, R₂, R₃, and R₄, are X₅, comprise X₅ as a substituent or X₅ is a substituent on any carbon atom in Q or in positions 1, 2, 3, 4, 5, 6, 7, 8, 9 and/or 10 of Formula VI.

In other illustrative embodiments of the present invention, there is provided a compound of any formula described herein wherein Q is selected from the group consisting of —CH₂—, —CY₁Y₂—, —CH₂CH₂—, —CY₁Y₂CY₃Y₄—, —CH₂CH₂CH₂—, and —CY₁Y₂CY₃Y₄CY₅Y₆—; where Y₁, Y₂, Y₃, Y₄, Y₅, and Y₆ are independently selected from the group consisting of: H, F, Br, Cl, I, OH, OR₅′, SH, any one group of Y₁/Y₂, Y₃/Y₄, and Y₅/Y₆ are ═O, and Y₁/Y₃ is an epoxide; and, at least one of Y₁, Y₂, Y₃, Y₄, Y₅ and Y₆ when present, is not H; and
R₅′ is a linear, branched, or cyclic, saturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated one to ten carbon alkyl group.

In other illustrative embodiments of the present invention, there is provided a compound of any formula described herein wherein Y₁, Y₂, Y₃, Y₄, Y₅ and Y₆, are H or halogen.

In other illustrative embodiments of the present invention, there is provided a compound of any formula described herein wherein Q is selected from the group consisting of: —CH₂—, —CH₂—CH₂—, —CH═CH, —CH₂—CH₂—CH₂— and —CH═CH—CH₂—.

In other illustrative embodiments of the present invention, there is provided a compound of any formula described herein wherein Q is selected from the group consisting of: —CH₂—, —CY₁Y₂—, —CH₂CH₂—, —CH═CH—, and —CY₁Y₂CY₃Y₄—.

In other illustrative embodiments of the present invention, there is provided a compound of Formula VII or a salt thereof:

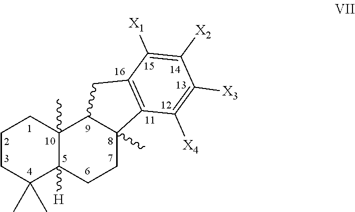

wherein;
X₁, X₂, X₃, and X₄ are independently selected from the group consisting of: H, X₅, R₆′, OH, —O—(C₁-C₁₀ alkyl), —CO₂H, —CO₂R₇′, F, Br, Cl, I, —CN, —SO₃H, —OSO₃H, NO₂, NH₂, —NHR₈′, and —N(R₉′)₂; where R₆′, R₈′ and R₉′ are independently X₅, or a linear, branched, or cyclic, saturated or unsaturated one to ten carbon alkyl group that is unsubstituted or is substituted with one or more of: X₅, OH, ═O, SH, F, Br, Cl, I, NH₂, —NHR₁₀′, —N(R₁₁′)₂, NO₂, —CO₂H, —CO₂R₁₂′, and epoxide;
R₇′, R₁₀′, R₁₁′, and R₁₂′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group; and
X₅ is a prodrug moiety and at least one of X₁, X₂, X₃ and X₄ are X₅, or comprise X₅.

In other illustrative embodiments of the present invention, there is provided a compound of Formula VIII or a salt thereof:

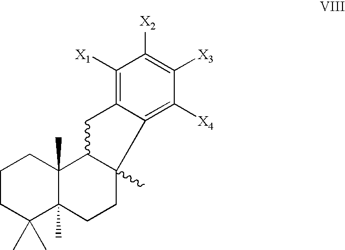

wherein;
X₁, X₂, X₃, and X₄ are independently selected from the group consisting of: H, X₅, R₆′, OH, —O—(C₁-C₁₀ alkyl), —CO₂H, —CO₂R₇′, F, Br, Cl, I, —CN, —SO₃H, —OSO₃H, NO₂, NH₂, —NHR₈′, and —N(R₉′)₂; where R₆′, R₈′ and R₉′ are independently X₅, or a linear, branched, or cyclic, saturated or unsaturated one to ten carbon alkyl group that is unsubstituted or is substituted with one or more of: X₅, OH, ═O, SH, F, Br, Cl, I, NH₂, —NHR₁₀′, —N(R₁₁′)₂, NO₂, —CO₂H, —CO₂R₁₂′, and epoxide;
R₇′, R₁₀′, R₁₁′, and R₁₂′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group; and
X₅ is a prodrug moiety and at least one of X₁, X₂, X₃ and X₄ are X₅, or comprise X₅.

In other illustrative embodiments of the present invention, there is provided a compound of any formula described herein wherein R₆′, R₇′, R₈′, and R₉′, in at least one of X₁, X₂, X₃, and X₄ is selected from the group consisting of: unsubstituted methyl, unsubstituted ethyl, unsubstituted propyl and unsubstituted butyl.

In other illustrative embodiments of the present invention, there is provided a compound of any formula described herein wherein at least one of X₁, X₂, and X₃, is selected from the group consisting of: H, X₅, R₆′, OH, O—(C₁-C₁₀ alkyl), halogen, —CONH₂, —CONHR₁₃′, —CO(R₁₄′)₂, NHR₈′ and N(R₉′)₂; where R₁₃′ and R₁₄′ are independently a linear, branched, or cyclic, saturated or unsaturated one to ten carbon alkyl unsubstituted or substituted with one or more of: OH, ═O, SH, F, Br, Cl, I, NH₂, —NHR₃″, —N(R₄″)₂, NO₂ and —CO₂H where R₃″ and R₄″ are independently a linear, branched, or cyclic, saturated or unsaturated one to ten carbon alkyl group.

In other illustrative embodiments of the present invention, there is provided a compound of any formula described herein wherein R₆′, R₈′, R₉′, R₁₃′, and R₁₄′, are selected from the group consisting of: unsubstituted methyl, unsubstituted ethyl, unsubstituted propyl and unsubstituted butyl.

In other illustrative embodiments of the present invention, there is provided a compound of any formula described herein wherein one or more of X₁, X₂, and X₃ are selected from the group consisting of: H, X₅, OH, O—(C₁-C₁₀ alkyl), —CONH₂, —CONHR₁₃′, and —CO(R₁₄′)₂, where R₁₃′ and R₁₄′ are independently a linear, branched, or cyclic, saturated or unsaturated one to ten carbon alkyl group that is unsubstituted or substituted with one or more of: OH, ═O, SH, F, Br, Cl, I, NH₂, —NHR₃″, —N(R₄″)₂, NO₂ and —CO₂H where R₃″ and R₄″ are independently a linear, branched, or cyclic, saturated or unsaturated one to ten carbon alkyl group.

In other illustrative embodiments of the present invention, there is provided a compound of any formula described herein wherein R₁₃′, and R₁₄′, are selected from the group consisting of: unsubstituted methyl, unsubstituted ethyl, unsubstituted propyl and unsubstituted butyl.

In other illustrative embodiments of the present invention, there is provided a compound of any formula described herein wherein one or more of X₁, X₂, and X₃ are selected from the group consisting of: H, X₅, OH, and OCH₃.

In other illustrative embodiments of the present invention, there is provided a compound of any formula described herein wherein X₄ is selected from the group consisting of: H, X₅, R₆′, OH, O—(C₁-C₁₀ alkyl), CO₂H and —CO₂R₇′.

In other illustrative embodiments of the present invention, there is provided a compound of any formula described herein wherein R₆′, and R₇′, are selected from the group consisting of: unsubstituted methyl, unsubstituted ethyl, unsubstituted propyl and unsubstituted butyl.

In other illustrative embodiments of the present invention, there is provided a compound of any formula described herein wherein X₄ is selected from the group consisting of: H, X₅, R₆′, OH, OCH₃, —CO₂H and —CO₂R₇′.

In other illustrative embodiments of the present invention, there is provided a compound of any formula described herein wherein X₄ is selected from the group consisting of: H, X₅, R₆′, OH, OCH₃, —CO₂H and —CO₂CH₃.

In other illustrative embodiments of the present invention, there is provided a compound of any formula described herein wherein X₅ comprises (a) a solubilizing moiety selected from the group consisting of: a moiety having one or more ionic entities at physiological pH; a moiety having multiple hydrogen bonding functionalities, such as —OH or amide; a monophosphate; a diphosphate; a triphosphate; a monosaccharide; an oligosaccharide; a polysaccharide; an oligopeptide; a polypeptide; an amino acid; an alpha amino acid a polyether and a combination thereof; and (b) a linking moiety selected from the group consisting of: —O—; —O—C(═O)—Z—; —NH—C(═O)—Z—; —CH₂C(═O)—; —C(═O)O—, —C(═O)HN—; where Z is a linear, branched, or cyclic, saturated or unsaturated one to ten carbon alkyl group that is unsubstituted or is substituted with one or more of: OH, ═O, SH, F, Br, Cl, I, NH₂, —NHR′, —NR′₂, NO₂, —CO₂H, —CO₂R′, and epoxide and individual carbon atoms may be replaced by S, O, N, NR′, or NR′₂ atoms; and each R′, is independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ are independently a linear, branched, or cyclic, saturated or unsaturated one to ten carbon alkyl group.

In other illustrative embodiments of the present invention, there is provided a compound of any formula described herein wherein X₅ comprises an amide linking moiety.

In other illustrative embodiments of the present invention, there is provided a compound of any formula described herein wherein X₅ comprises an ester linking moiety.

In other illustrative embodiments of the present invention, there is provided a compound of any formula described herein wherein X₅ comprises a solubilizing moiety comprising an NH₂ moiety.

In other illustrative embodiments of the present invention, there is provided a compound of any formula described herein wherein X₅ comprises an amino acid.

In other illustrative embodiments of the present invention, there is provided a compound of any formula described herein wherein X₅ comprises a phosphate.

In other illustrative embodiments of the present invention, there is provided a compound of any formula described herein wherein X₅ comprises a polyethylene glycol moiety.

In other illustrative embodiments of the present invention, there is provided a pharmaceutical composition comprising a compound of any formula described herein and a pharmaceutically acceptable excipient.

In other illustrative embodiments of the present invention, there is provided a compound of any formula described herein or a pharmaceutical composition described herein for the treatment or prophylaxis of an inflammatory, neoplastic, hematopoetic or immune disorder or condition.

In other illustrative embodiments of the present invention, there is provided a use of a compound of any formula described herein for the treatment or prophylaxis of an inflammatory, neoplastic, hematopoetic or immune disorder or condition. The use may be for preparation of a medicament.

In other illustrative embodiments of the present invention, there is provided a method of prophylaxis or treatment of an immune, hematopoietic, inflammatory or neoplastic disorder or condition comprising administering to a patient in need of said prophylaxsis or treatment, an effective amount of a pharmaceutical composition described herein.

In other illustrative embodiments of the present invention, there is provided a use or a method as described herein wherein the neoplastic condition is a blood cancer, multiple myeloma, chronic myeloid leukemia, or acute myelogenous leukemia.

In other illustrative embodiments of the present invention, there is provided a use or a method as described herein wherein the immune disorder is an autoimmune disorder.

In other illustrative embodiments of this invention there is provided a method of making or a method of synthesizing a compound as described herein.

In other illustrative embodiments of this invention there is provided a method of prophylaxis or treatment of an immune, hematopoietic, inflammatory or neoplastic disorder or condition comprising administering to a patient in need of said prophylaxsis or treatment, an effective amount of a pharmaceutical composition as described above. Such compositions may comprise previously known compounds of a formula described herein which have not been known as biologically active compounds suitable for pharmaceutical use or not been known as particularly efficacious.

In other illustrative embodiments of this invention there is provided the use of a compound described above or pharmaceutically acceptable salt thereof for modulation of SHIP 1 activity and for preparation of agents and medicaments for the modulation of SHIP 1 activity. Such modulation may be in vitro or in vivo. Agents for in vivo use include a pharmaceutical composition of this invention as well as agents adapted for in vitro use. The modulation may be for a treatment or prophylaxis of an immune, inflammatory, or neoplastic condition or disorders as described above.

### BRIEF DESCRIPTION OF THE DRAWINGS

FIG. 1: is a graph depicting the results of a cell based assay to test relative inhibition of TNFα by a prodrug compound, Compound 103, compared to a non-prodrug compound, Compound 100.

FIG. 2: is a graph depicting the results of a cell based assay to test the inhibition of macrophage TNFα production by varying concentrations of a prodrug, Compound 106.

FIG. 3: is a graph depicting the results of a cell based assay to test the inhibition of calcium influx in mast cells by a prodrug, Compound 106.

FIG. 4: is a graph depicting the results of a cell based assay to test the inhibition of TNFα production in wild type (WT) and knock-out (KO) macrophages by a prodrug, Compound 108.

FIG. 5A: is a graph depicting the results of the ability of Compound 100 at varying concentrations to reduce tumor cell survival in multiple myeloma (MM) cell lines.

FIG. 5B: is a graph depicting the results of the ability of Compound 100 at varying concentrations to reduce tumor cell survival in multiple myeloma (MM) cell lines.

FIG. 5C: is a graph depicting the results of the ability of AQX-016A at varying concentrations to reduce tumor cell survival in multiple myeloma (MM) cell lines.

FIG. 6A: is a graph depicting the results of the ability of compound 100 at varying concentrations to inhibit growth of OPM2 MM cell lines.

FIG. 6B: is a graph depicting the results of the ability of compound 100 at varying concentrations to inhibit growth of MM.1S MM cell lines.

FIG. 6C: is a graph depicting the results of the ability of AQX-016A at varying concentrations to inhibit growth of RPMI 8226 MM cell lines.

FIG. 6D: is a graph depicting the results of the ability of AQX-016A at varying concentrations to inhibit growth of U266 MM cell lines.

FIG. 6E: is a graph depicting the results of the ability of AQX-016A at varying concentrations to inhibit growth of LCC6-Her2 MM cell lines.

FIG. 7A: is a graph depicting the results of the activation of SHIP enzyme in vitro of Compound 100, AQX-16A and Compound 103.

FIG. 7B: is a graph depicting the results of the activation of SHIP enzyme in vitro of Compound 100 and AQX-16A.

FIG. 7C: is a graph depicting the results of Compound 100 inhibiting TNFα production from LPS stimulated SHIP^{+/+} but not BMmφs.

FIG. 7D: is a graph depicting the results of Compound 100 inhibiting LPS-induced plasma TNFα levels in mice.

FIG. 8A: is a graph depicting the results of SHIP^{+/+} ( [symbol] ) and SHIP^{-/-} ( [symbol] ) macrophages pretreated with AQX-016A or carrier 30 min prior to stimulation with 10 ng/mL of LPS at 37° C. for 2 h and TNFα production determination by ELISA. Absolute TNFα levels for SHIP^{+/+} and SHIP^{-/-} cells were 623+/−30 and 812+/−20 pg/ml, respectively. Data are expressed as mean+/SEM and are representative of three independent experiments.

FIG. 8B: is a graph depicting the results of SHIP^{+/+} and SHIP^{-/-} mast cells pre-loaded with IgE and Fura-2 and treated for 30 min with 15 μM AQX-016A or carrier. Cells were then stimulated (as indicated by the arrow) with 0 ( [symbol] ) or 10 ( [symbol] ) ng/mL DNP-HSA and intracellular calcium levels monitored over time by spectrofluorometry.

FIG. 9: is a graph depicting the results of mice administered 20 mg/kg AQX-016A or 0.4 mg/kg dexamethasone orally 30 min prior to an IP injection of 2 mg/kg LPS. Blood was collected 2 h later for TNFα determination by ELISA. Each symbol indicates one mouse and data are representative of three independent experiments.

FIG. 10A: is a graph depicting the results of Compound 100 inhibiting DNFB-induced neutrophil-specific myeloperoxidase (MPO) in sensitized mice. P-value <0.02 for the Compound 100 vs the vehicle treated groups. All data are representative of three independent experiments. Data are representative of three independent experiments.

FIG. 10B: is a graph depicting the results of AQX-016A inhibiting mast cell degranulation in CD1 mice sensitized to hapten DNP by cutaneous application.

FIG. 11A: is a graph depicting the results of SHIP enzyme initial velocities at the indicated concentration of inositol-1,2,4,5-tetrakisphosphate (IP₄) substrate.

FIG. 11B: is a graph depicting the results of the ability of product PI-3,4-P₂ (20 μM) or Compound 100 (3 μM) to activate wild-type (WT) and C2 domain deleted (ΔC2) SHIP enzyme at 30 μM IP₄.

FIG. 11C: is a graph depicting the results of a protein overlay assay in which recombinant C2 domain was pre-incubated for 30 min at 23° C. with 4. μM of Compound 100 or EtOH control and allowed to bind to PI-3,4-P₂ immobilized on membrane strips.

FIG. 11D: is a graph depicting the results of bead associated radioactivity obtained from recombinant C2 domain (10 nM) coated onto Copper chelate (His-Tag) YSi SPA Scintillation Beads in the presence of 0.25% BSA and incubated with 5 μCi of [³H]-Compound 100. Data are expressed as mean+/SEM and are representative of at least three independent experiments.

FIG. 11E: is a graph depicting the results of bead associated radioactivity obtained from copper chelate (His-Tag) YSi SPA Scintillation Beads coated with either wild-type (WT) or C2 domain deleted (ΔC2) SHIP enzyme in the presence of 0.25% BSA aliquoted into 96 well plates and incubated with 5 μCi of [³H]-Compound 100 (42 Ci/mmol) with shaking at 23° C. in the dark. The amount of [³H]-Compound 100 interacting with the protein coated beads was quantified on a plate scintillation counter.

FIG. 12A: is a graph depicting the results of the activity of the enzymes in the presence of Compound 100 compared to that in the vehicle control and expressed as a % change in activity relative to that observed in the vehicle control. Changes in activity of <25% were not considered significant.

FIG. 12B: is a graph depicting the results of the activity of enzymes affected by Compound 100 by more the 25% as shown in FIG. 12A.

FIG. 13: is a graph depicting the results of the effect of Compound 100 and vehicle control on tumour size in mice.

FIG. 14: is a graph depicting the results of the effect of Compound 100 and vehicle control on tumour volume over time in mice.

### DETAILED DESCRIPTION

In this specification, the following abbreviations will appear: THF (tetrahydrofuran); n-buLi (n-butyllithium); t-buLi (tert-butyllithium); Ph₃PMe (methyl triphenyl phosphonium bromide); PCC (pyridinium chlorochromate); Ac (acetyl); Me (methyl); Et (ethyl); prop. (propyl); but. (butyl); RT, rt, or, r.t. (room temperature); hr. (hour(s)); DMSO (dimethylsulfoxide); DNFB (2,4-dinitrofluorobenzene); LPS (lipopolysaccarhide); TNF-α (Tumor Necrosis Factor Alpha); TBS (tert-butyl dimethylsilyl); EA (ethyl acetate); PG (protecting group); AA (amino acid); DCM (dichloromethane); DIPC (1,3-diisopropylcarbodiimide); DMAP (Dimethylamino pyridine); TFA (Trifluoroacetic acid); PEG (Polyethylene glycol); and BOC (t-Butyl carbamate).

As used herein the phrase “alkyl” refers to a molecule comprising hydrogen and carbon having the general formula CₙH₂ₙ₊₁. A “Cₓ to C_{y} alkyl” or a “Cₓ-C_{y} alkyl” refers to an alkyl having a number of carbons, the number being from x to y carbons. For example, “C₁ to C₆ alkyl” denotes that the alkyl may have 1, 2, 3, 4, 5 or 6 carbons.

All possible stereoisomers, epimers, diastereomers and enantiomers and mixtures thereof are specifically included by formulas described herein that have one or more chiral centers with a “ [symbol] ” bond (hereinafter termed a stereo-bond). Stereo-bonds denote that any one or more of the possible orientations of the bond is/are specifically included or specifically excluded from a particular embodiment and all of the embodiments, when considered together, include all such combinations of inclusion and exclusion of the possible bond orientations.

The phrase “stereo-mixture” as used herein may be a mixture of equal quantities or unequal quantities of two or more different stereoisomers. Stereo-mixtures may comprise any particular stereoisomer from 0% to 100% (and all values in between) as a component of the stereo-mixture, provided that at least 2 different stereoisomers are present in the mixture. A “racemic mixture” is a stereo-mixture that has equal quantities of each of the stereoisomers contained in the mixture.

The phrase “stereo-pure compound” as used herein refers to a compound having one or more chiral centers wherein each and every molecule of the compound has the same stereochemical structure. The phrase “substantially stereo-pure compound” refers to a compound that may be a stereo-pure compound or may be a compound wherein at least 97% of the molecules have the same stereochemical structure. Substantially stereo-pure compounds may be compounds wherein at least 98% of the molecules have the same stereochemical structure or may be compounds wherein at least 99% of the molecules have the same stereochemical structure. Substantially stereo-pure compounds may be compounds wherein at least 99.5% of the molecules have the same stereochemical structure or may be compounds wherein at least 99.9% of the molecules have the same stereochemical structure.

As used herein, the structure:

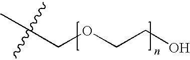

defines a polyethyleneglycol moiey (PEG) where n is the number of repeating units in the PEG.

As used herein, the structure:

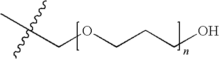

defines a polypropyleneglycol moiey (PPG) where n is the number of repeating units in the PPG.

### SHIP 1 Modulating Compounds and Prodrugs

Compounds of the invention comprise a pelorol, homopelorol, or pelorol/homopelorol analog core joined to a solubilizing moiety. The core and solubilizing moiety may be joined through a linking moiety.

In some embodiments, the core is or derived from a compound of Formula I or a salt thereof,

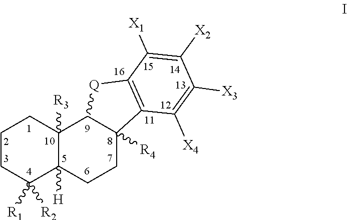

wherein;
R₁ and R₂ are independently selected from the group consisting of: H—CH₃, —CH₂CH₃, —CH₂OH, —CH₂OR′, —CHO, —CO₂H, and —CO₂R′;
R₃ and R₄ are independently selected from the group consisting of: H, —CH₃, —CH₂CH₃, —CH₂OH, —CH₂OR′, —CHO, —CO₂H, and —CO₂R′;
Q is a carbon skeleton selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂—, and —CY₁Y₂CY₃Y₄CY₅Y₆—; where Y₁, Y₂, Y₃, Y₄, Y₅, and Y₆ are independently selected from the group consisting of: H, F, Br, Cl, I, OH, OR′, and SH; or any one group of Y₁/Y₂, Y₃/Y₄, and Y₅/Y₆ may be ═O; or Y₁/Y₃ may form an epoxide; and, at least one of Y₁, Y₂, Y₃, Y₄, Y₅ and Y₆ when present, is not H;
X₁, X₂, X₃, and X₄ are independently selected from the group consisting of: H, R, OH, —O—(C₁-C₁₀ alkyl), —CO₂H, —CO₂R′, F, Br, Cl, I, —CN, —SO₃H, —OSO₃H, NO₂, NH₂, —NHR, and —N(R)₂; where R is a linear, branched, or cyclic, saturated or unsaturated one to ten carbon alkyl group that is unsubstituted or is substituted with one or more of: OH, ═O, SH, F, Br, Cl, I, NH₂, —NHR′, —NR′₂, NO₂, —CO₂H, —CO₂R′, and epoxide;

and R′ is a linear, branched, or cyclic, saturated or unsaturated one to ten carbon alkyl group that is unsubstituted or substituted with one or more of: OH, ═O, SH, F, Br, Cl, I, NH₂, —NHR″, —NR″₂, NO₂ and —CO₂H where R″ is a linear, branched, or cyclic, saturated or unsaturated one to ten carbon alkyl group.

Compounds of Formula I have chiral centres at C-5, C-8, C-9 and C-10 and may be chiral at C-4 depending upon whether R₁ and R₂ are different. Some embodiments have the same relative configuration of chiral centres as does pelorol or are enantiomers thereof, namely: S, R, R, S; or R, S, S, R (at C-5, 8, 9 and 10 respectively). Some embodiments have the same absolute configuration as pelorol at chiral centres. Some embodiments have the same relative configuration as pelorol at C-5 and C-10 with independently variable configurations at C-8 and C-9. Some embodiments have the same relative configuration as pelorol at C-5, C-8, and C-10 with variable configuration at C-9. In all cases, the configuration at C-4 (if chiral) may be variable or may be the same relative configuration to the remaining chiral centres as is shown in examples of structures of compounds of Formula I illustrated herein.

In various embodiments the core may have more specific limitations with respect to substituents Q, R₁, R₂, R₃, R₄, X₁ X₂, X₃ and X₄. Any combination of the following limitations is encompassed by this invention.
(a) Q may be as defined for Formula I except that Y₁, Y₂, Y₃, Y₄, Y₅ and Y₆, is limited to H or halogen;
(b) Q may be limited to —CH₂—, —CH₂—CH₂—, —CH═CH, —CH₂—CH₂—CH₂— and —CH═CH—CH₂—;
(c) Q may be limited to or saturated moieties in the limitation of Formula I, or according to the limitations of paragraph (a) or (b) above;
(d) Q may be limited to a one or two carbon skeleton within the limitations of Formula I, or according to the limitations of any of paragraphs (a) to (c) above;
(e) one or both of R₁ and R₂ may be limited to —CH₃, —CH₂CH₃, —CH₂OH, —CH₂OR′, —CHO, —CO₂H, and —CO₂R′;
(f) one or both of R₁ and R₂ may be limited to methyl, ethyl, —CH₂OH or —CH₂OR′;
(g) R′ in one or both of R₁ and R₂ according to Formula I, or the limitation of paragraph (f) above, may be limited to methyl, ethyl, propyl or butyl;
(h) one or both of R₁ and R₂ may be limited to methyl or ethyl;
(i) one or both of R₁ and R₂ may be limited to methyl;
(j) R and R′ in any one or more of X₁-X₄ may be limited to unsubstituted methyl, ethyl, propyl or butyl;
(k) one or more of X₁-X₃ may be limited to H, R, OH, O—(C₁-C₁₀ alkyl), halogen, —CONH₂, —CONHR′, —COR′₂, NHR or N(R)₂ where R and R′ are limited as in Formula I, or R and R′ may be according to paragraph (j) above;
(l) one or more of X₁-X₃ is limited to H, OH, O—(C₁-C₁₀ alkyl), —CONH₂, —CONHR′, and —COR′₂, where R and R′ are as in Formula I, or R and R′ may be limited according to paragraph (j) above;
(m) one or more of X₁-X₃ may be limited to H, OH, and OCH₃;
(n) X₄ may be limited to H, R, OH, O—(C₁-C₁₀ alkyl), CO₂H or —CO₂R′, with R and R′ as in Formula I, or R and R′ may be limited according to paragraph (i) above;
(o) X₄ may be limited to H, R, OH, OCH₃, —CO₂H and —CO₂R′ with R and R′ limited according to paragraph (j) above; and,
(p) X₄ may be limited to H, R, OH, OCH₃, —CO₂H or —CO₂CH₃.

In some embodiments, the core is or derived from a compound of Formula 1A or a salt thereof:

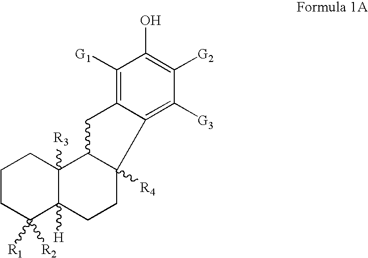

wherein;
R₁ and R₂ are independently selected from the group consisting of: —CH₃, —CH₂CH₃, —CH₂OH, —CH₂OR₁′, —CHO, —CO₂H, and —CO₂R₂′;
R₃ and R₄ are independently selected from the group consisting of: H, —CH₃, —CH₂CH₃, —CH₂OH, —CH₂OR₃′, —CHO, —CO₂H, and —CO₂R₄′;
R₁′, R₂′, R₃′, and R₄′, are independently a linear, branched, or cyclic, saturated or unsaturated one to ten carbon alkyl group that is unsubstituted or substituted with one or more of: OH, ═O, SH, F, Br, Cl, I, NH₂, —NHR₁″, —N(R₂″)₂, NO₂ and —CO₂H where R₁″ and R₂″ is a linear, branched, or cyclic, saturated or unsaturated one to ten carbon alkyl group;
G₁ is selected from the group consisting of: O—(C₁-C₁₀ alkyl) and H;
G₂ is H or C₁-C₁₀ alkyl; and
G₃ is selected from the group consisting of: H, —OH, C₁-C₁₀ alkyl and O—(C₁-C₁₀ alkyl).

In other embodiments of Formula 1A, G₁ is selected from the group consisting of —O-methyl and H; G₂ is H or methyl; and G₃ is selected from the group consisting of: H, methyl and O-methyl.

Compounds of Formula 1A have chiral centres at C-5, C-8, C-9 and C-10 and may be chiral at C-4 depending upon whether R₁ and R₂ are different. Some embodiments have the same relative configuration of chiral centres as does pelorol or are enantiomers thereof, namely: S, R, R, S; or R, S, S, R (at C-5, 8, 9 and 10 respectively). Some embodiments have the same absolute configuration as pelorol at chiral centres. Some embodiments have the same relative configuration as pelorol at C-5 and C-10 with independently variable configurations at C-8 and C-9. Some embodiments have the same relative configuration as pelorol at C-5, C-8, and C-10 with variable configuration at C-9. In all cases, the configuration at C-4 (if chiral) may be variable or may be the same relative configuration to the remaining chiral centres as is shown in examples of structures of compounds of Formula 1a illustrated herein.

In various embodiments the pelorol analog may have more specific limitations with respect to substituents R₁, R₂, R₃, and R₄. Any combination of the following limitations is encompassed by this invention.
(a) one or both of R₁ and R₂ may be limited to methyl, ethyl, —CH₂OH, —CH₂OR₁′, or —CH₂OR₃′;
(b) R₁′, R₂′, R₃′, and/or R₄′, in one or both of R₁ and R₂ according to Formula 1a, or in the limitation of paragraph (a) above, may be limited to methyl, ethyl, propyl or butyl;
(c) one or both of R₁ and R₂ may be limited to methyl or ethyl;
(d) one or both of R₁ and R₂ may be limited to methyl;

In some embodiments, the core is or derived from a compound of Formula 2A or a salt thereof:

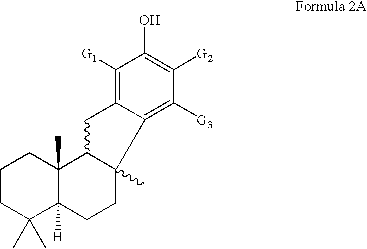

wherein;
G₁ is selected from the group consisting of: O—(C₁-C₁₀ alkyl) and H;
G₂ is H or C₁-C₁₀ alkyl; and
G₃ is selected from the group consisting of: H, —OH, C₁-C₁₀ alkyl and O—(C₁-C₁₀ alkyl).

Some embodiments of the core described above are encompassed by the invention. In some embodiments of the core described above, the core comprises the solubilizing moiety and linking moiety and no further modification to the core is required. Whether or not a particular core requires further modification may be determined on the basis that if it comprises a solubilizing and a linking moiety as described below, then no further modification will be required. Nevertheless, additional solubilizing moieties may or may not be desirable and therefore further modification may be applied to all embodiments of the core described above. In some embodiments a core without a linking moiety and/or a solubilizing moiety are within the scope of the present invention.

Solubilizing moieties may be any moiety having one or more ionic entities at physiological pH or multiple hydrogen bonding functionalities such as —OH or amide. Non-limiting examples of solubilizing moieties may be selected from: monophosphates; diphosphates; triphosphates; monosaccharides; oligosaccharides; polysaccharides; oligopeptides, including, but not limited to, dipeptides and tripeptides; polypeptides; amino acids; alpha amino acids —CH(NH₂)-(AA); polyethers and combinations thereof, wherein (AA) is an amino acid side chain.

Amino acid side chains include, but are not limited to, those portions of the naturally occurring protein amino acids and non-naturally occurring amino acids that do not comprise the alpha-carbon, the alpha-amine, the alpha-carboxy group and the hydrogen bonded directly to the alpha-carbon.

Polyethers include, but are not limited to, polyethylene glycol (PEG), methylated-polyethylene glycol (MPEG), polypropylene glycol, PEG-amine and MPEG-amine. Polyethers, including all of those specifically mentioned above, may have molecular weights of from about 62 to about 20,000 or more and all possible variations in between. Such a range of molecular weights generally corresponds to the repeating oxyalkane unit have from between about 0 to about 450 repeats. Polyethers, including all of those specifically mentioned above, may have molecular weights of from about 62 to about 6,500 or more and all possible variations in between. Such a range of molecular weights generally corresponds to the repeating oxyalkane unit have from between about 0 to about 150 repeats. From between about 1 repeat to about 50 repeats of the oxyalkane unit is commonly used and this represents a molecular weight range of from about 100 to about 2500.

In particular the following structures in Table I are non-limiting examples of solubilizing moieties. In the following table, each R is independently a linear, branched, or cyclic, saturated or unsaturated one to ten carbon alkyl group that is unsubstituted or is substituted with one or more of: OH, ═O, SH, F, Br, Cl, I, NH₂, —NHR′, —NR′₂, NO₂, —CO₂H, —CO₂R′, and epoxide;

and R′ is a linear, branched, or cyclic, saturated or unsaturated one to ten carbon alkyl group that is unsubstituted or substituted with one or more of: OH, ═O, SH, F, Br, Cl, I, NH₂, —NHR″, —NR″₂, NO₂ and —CO₂H where R″ is a linear, branched, or cyclic, saturated or unsaturated one to ten carbon alkyl group.

TABLE I
SOLUBILIZING MOIETIES

| | |
|---|---|
| 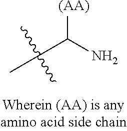 | 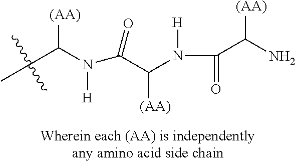 |
| 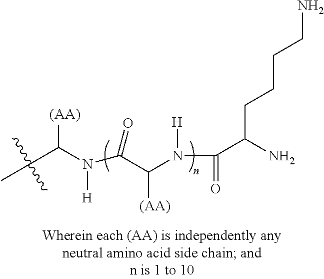 | 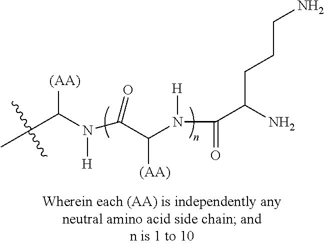 |
| 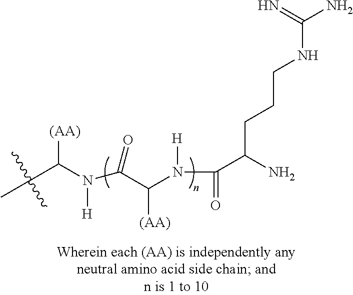 | 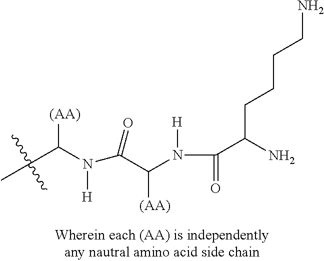 |
| 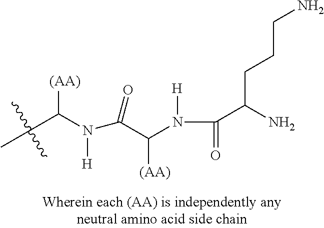 | 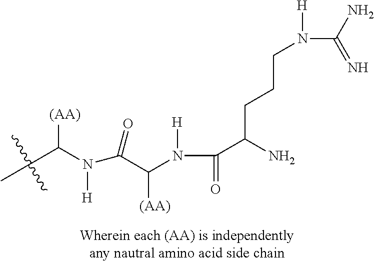 |
| 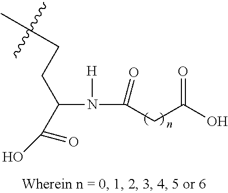 | 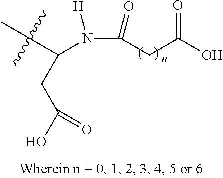 |
| 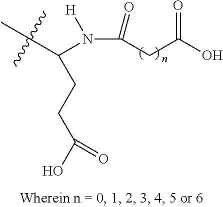 | 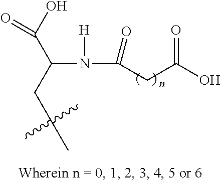 |
| 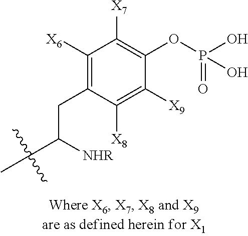 | 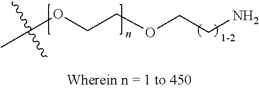 |
| 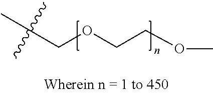 | 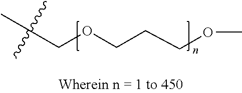 |
| 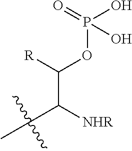 | 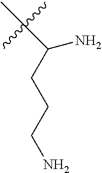 |
| 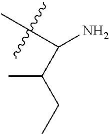 | 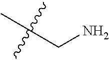 |
| 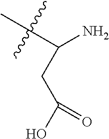 | 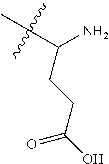 |
| 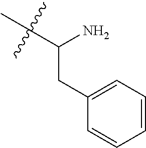 | 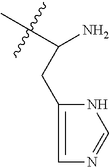 |
| 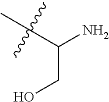 | 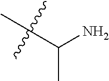 |
| 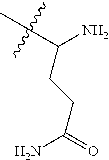 | 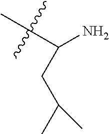 |
| 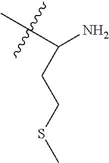 | 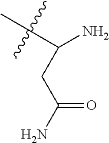 |
| 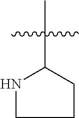 | 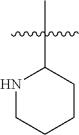 |
| 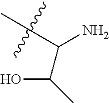 | 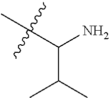 |
| 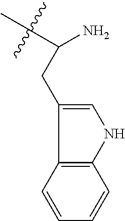 | 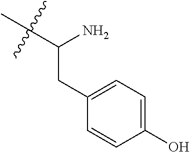 |
| 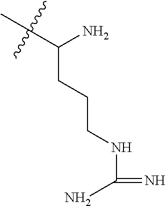 | 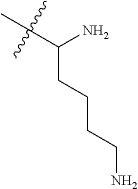 |
| 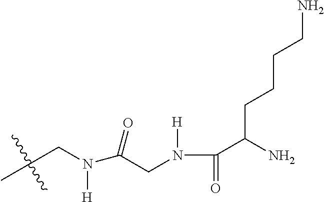 | 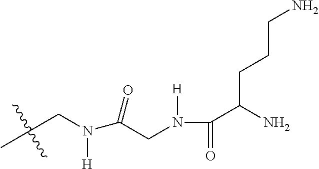 |
| 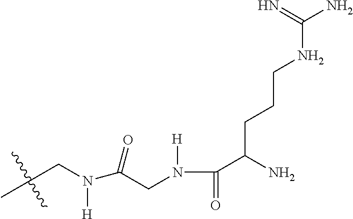 | 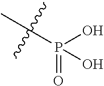 |
| 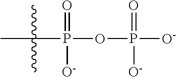 | 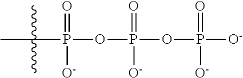 |
| 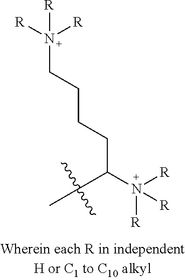 | 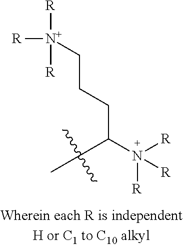 |
| 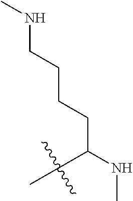 | 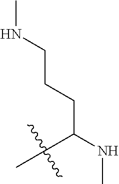 |
| 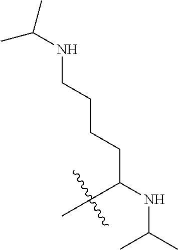 | 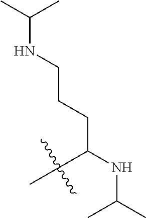 |
| 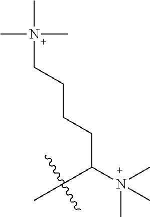 | 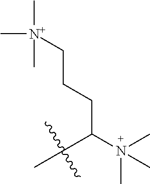 |

In some particular embodiments, each R as set out in Table I may be independently selected from H, methyl or acyl.

Linking moieties may connect the core to a solubilizing moiety. A linking moiety is a moiety that is cleaved in vivo such that a compound of the core is produced via cleavage of the linking moiety from the core. In some embodiments, cleavage of the linking moiety may be related to the stability of the linking moiety under physiological conditions. In some embodiments, the linking moiety may be cleaved in vivo enzymatically. In some embodiments, cleavage of the linking moiety in vivo results in the formation of a core comprising an OH moiety where the linking moiety was bonded to the core prior to cleavage. Linking moieties comprising an ester moiety may provide formation of a core comprising an OH moiety where the ester linking moiety was bonded to the core prior to cleavage. A Linking moiety may be selected from the following moieties: —O—C(═O)—Z—, —NH—C(═O)—Z—, —CH₂C(═O)—, —C(═O)O—, and —C(═O)HN—; where Z is a linear, branched, or cyclic, saturated or unsaturated one to ten carbon alkyl group that is unsubstituted or is substituted with one or more of: OH, ═O, SH, F, Br, Cl, I, NH₂, —NHR′, —NR′₂, NO₂, —CO₂H, —CO₂R′, and epoxide and individual carbon atoms may be replaced by S, O, N, NR′, or NR′₂ atoms; and wherein each R′ is independently a linear, branched, or cyclic, saturated or unsaturated one to ten carbon alkyl group that is unsubstituted or is substituted with one or more of: OH, ═O, SH, F, Br, Cl, I, NH₂, —NHR″, —NR″₂, NO₂, —CO₂H, —CO₂R″, and epoxide; and R″ is a linear, branched, or cyclic, saturated or unsaturated one to ten carbon alkyl group. In the case of phosphates, —O— is also a suitable linking moiety. Specific non-limiting examples of linking moieties are described below In Table II, where 1 represents the point of attachment to the core and 2 represents the point of attachment to a solubilizing moiety:

TABLE II
LINKING MOIETIES

| | |
|---|---|
| 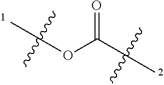 | 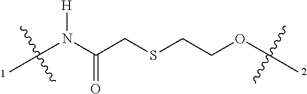 |
| 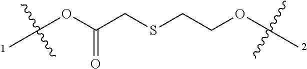 | 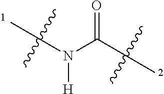 |
| 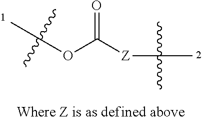 | 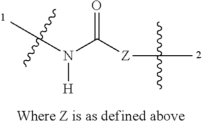 |
| 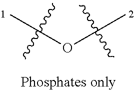 | |

The linking moiety and the solubilizing moiety may also be described as a single structure, termed a prodrug moiety or X₅. Prodrug moieties provide for improved solubility of core compounds. Additionally prodrugs may provide for better activity in vivo that the core compound that is produced upon cleavage of the prodrug moiety compared to direct administration of the core.

The prodrug moiety comprises all that is added to the core such that a compound of this invention is formed. Any combination of any linking moiety as described herein bonded to any solubilizing moiety as described herein may comprise a prodrug moiety. In some embodiments, a prodrug moiety is stable and difficult to remove from the core. In some embodiments, prodrug moieties may be moieties that may be cleaved in vivo such that a compound of the core is produced via cleavage at the linking moiety thereby separating the prodrug moiety or the solubilizing moiety from the core. In some embodiments, the linking moiety may be cleaved enzymatically. In some embodiments, in vivo cleavage of the linking moiety to separate the prodrug moiety or solubilizing moiety from the core results in the formation of a core comprising an OH moiety where the prodrug moiety was bonded to the core prior to cleavage. Prodrug moieties comprising an ester moiety may provide formation of a core comprising an OH moiety where the ester prodrug moiety was bonded to the core prior to cleavage. Specific, non-limiting examples of prodrug moieties are described below in Tables III and IV. In the following Table III, each R is independently a linear, branched, or cyclic, saturated or unsaturated one to ten carbon alkyl group that is unsubstituted or is substituted with one or more of: OH, ═O, SH, F, Br, Cl, I, NH₂, —NHR′, —NR′₂, NO₂, —CO₂H, —CO₂R′, and epoxide;
and R′ is a linear, branched, or cyclic, saturated or unsaturated one to ten carbon alkyl group that is unsubstituted or substituted with one or more of: OH, ═O, SH, F, Br, Cl, I, NH₂, —NHR″, —NR″₂, NO₂ and —CO₂H where R″ is a linear, branched, or cyclic, saturated or unsaturated one to ten carbon alkyl group.

TABLE III
PRODRUG MOIETIES

| | |
|---|---|
| 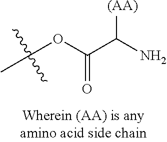 | 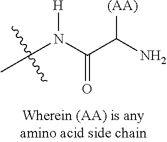 |
| 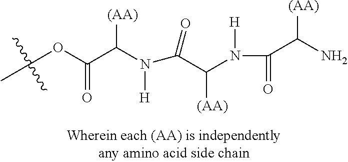 | 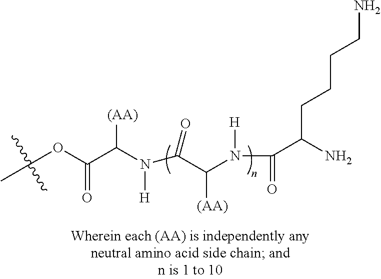 |
| 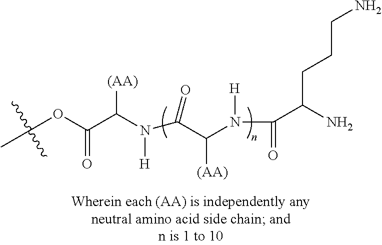 | 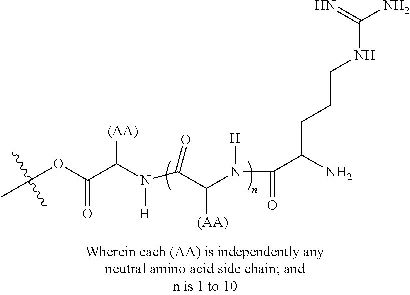 |
| 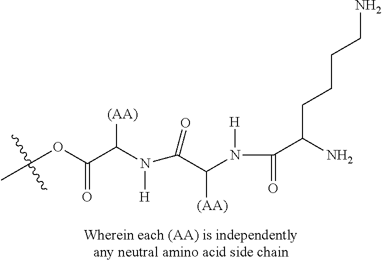 | 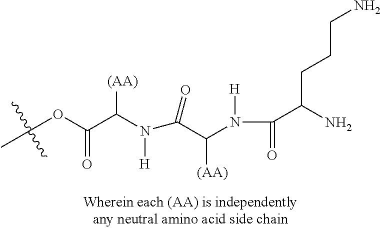 |
| 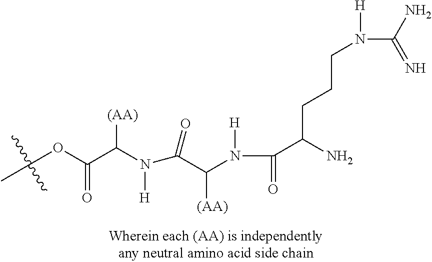 | 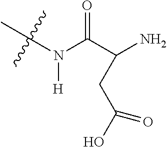 |
| 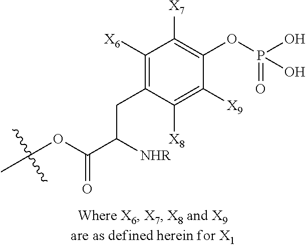 | 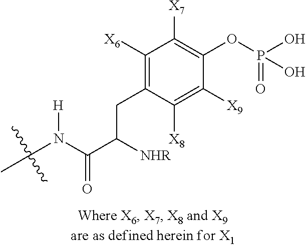 |
| 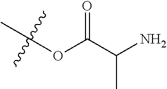 | 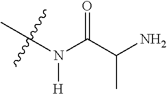 |
| 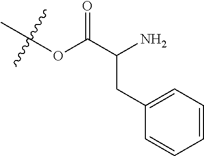 | 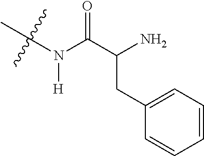 |
| 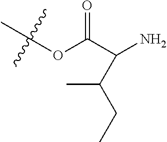 | 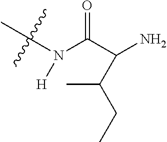 |
| 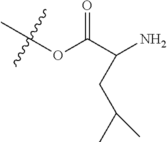 | 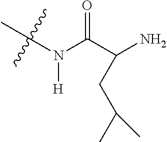 |
| 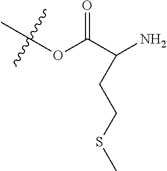 | 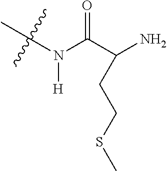 |
| 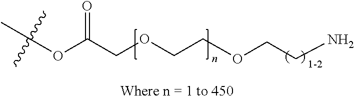 | 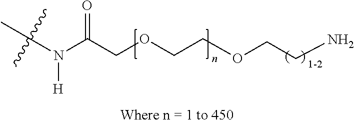 |
| 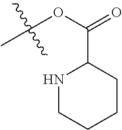 | 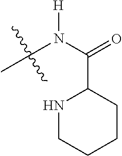 |
| 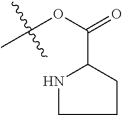 | 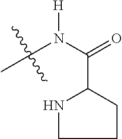 |
| 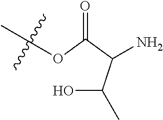 | 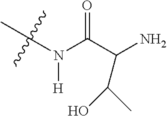 |
| 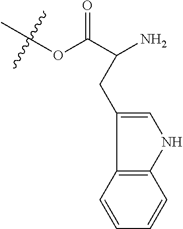 | 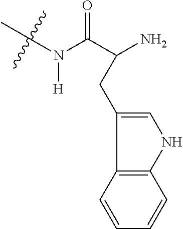 |
| 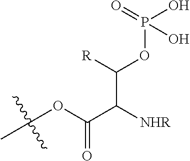 | 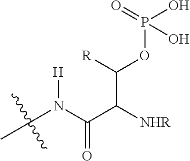 |
| 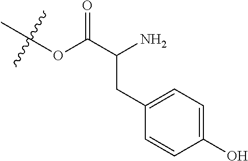 | 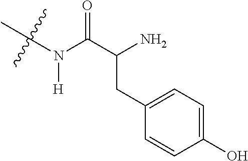 |
| 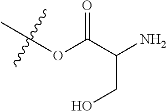 | 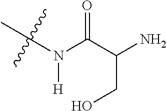 |
| 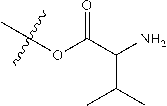 | 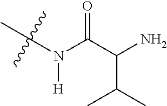 |
| 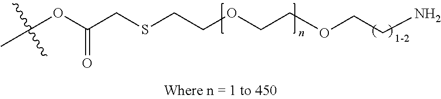 | 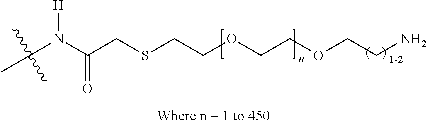 |
| 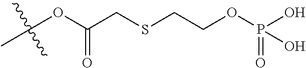 | 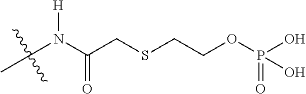 |
| 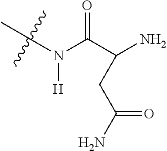 | 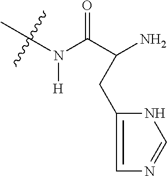 |
| 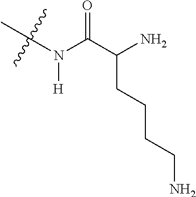 | 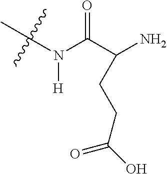 |
| 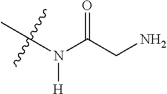 | 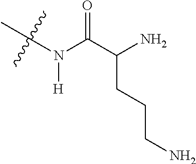 |
| 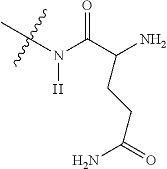 | 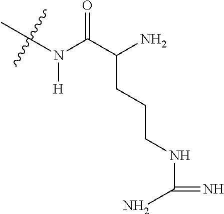 |
| 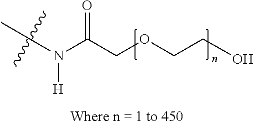 | 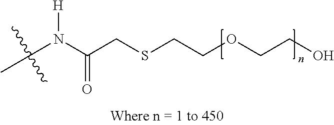 |
| 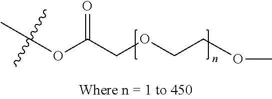 | 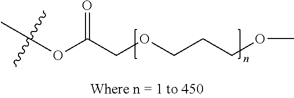 |
| 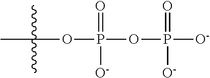 | 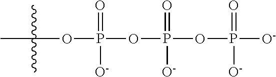 |
| 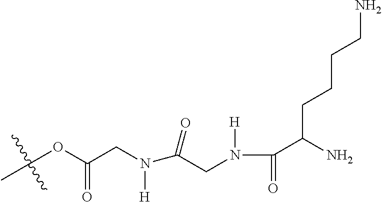 | 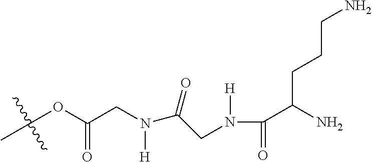 |
| 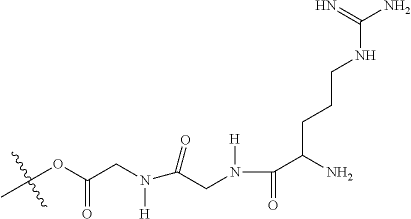 | |

In some particular embodiments, each R as set out in Table III may be independently selected from H, methyl or acyl.

TABLE IV
ESTER PRODRUG MOIETIES

| | |
|---|---|
| 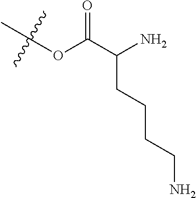 | 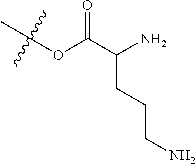 |
| 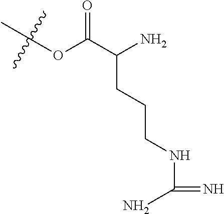 | 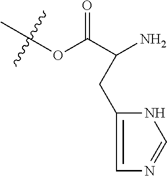 |
| 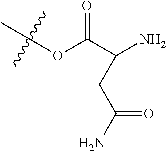 | 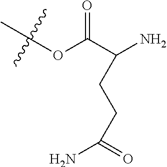 |
| 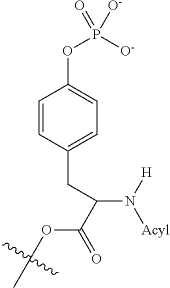 | 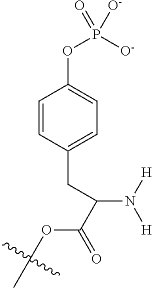 |
| 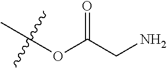 | 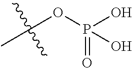 |
| 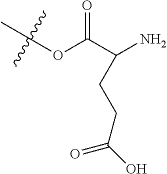 | 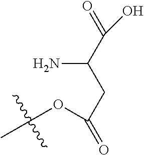 |
| 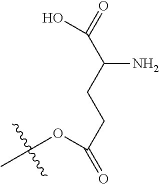 | 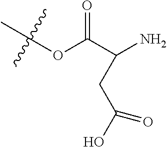 |
| 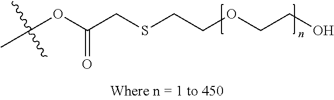 | 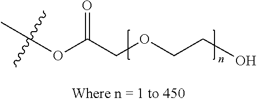 |
| 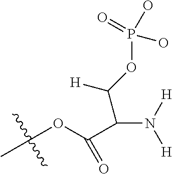 | 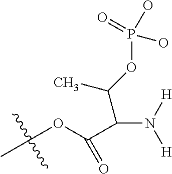 |
| 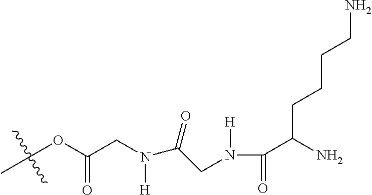 | 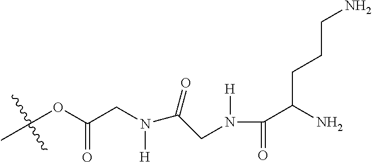 |
| 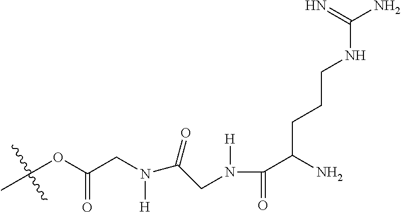 | 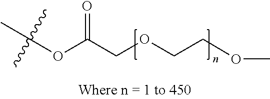 |
| 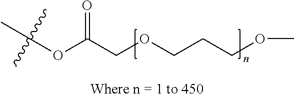 | 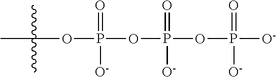 |
| 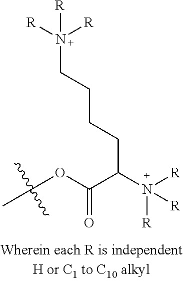 | 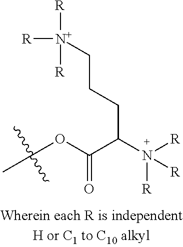 |
| 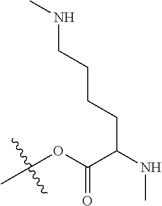 | 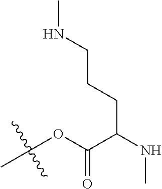 |
| 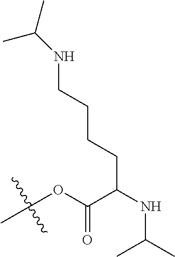 | 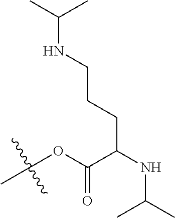 |
| 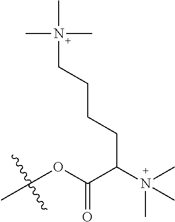 | 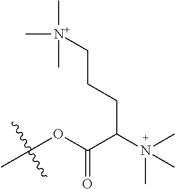 |

Prodrug moieties may be added to the core by replacing at least one of X₁, X₂, X₃, X₄, R₁, R₂, R₃ or R₄ with a prodrug moiety, or by adding a prodrug moiety onto an existing X₁, X₂, X₃, X₄, R₁, R₂, R₃, or R₄ substituent by substituting an atom or group of atoms from the existing X₁, X₂, X₃, X₄, R₁, R₂, R₃, or R₄ substituent with the prodrug moiety. The atom or group of atoms that may be substituted from the existing X₁, X₂, X₃, X₄, R₁, R₂, R₃, or R₄ substituent may be at a location close to the core or distanced from the core, in the main chain of the existing X₁, X₂, X₃, X₄, R₁, R₂, R₃, or R₄ substituent, or in a side chain of the existing X₁, X₂, X₃, X₄, R₁, R₂, R₃, or R₄ substituent. Prodrug moieties may also be added or substituted on to any one or more of the carbon atoms in Q or at positions 1, 2, 3, 4, 5, 6, 7, 8, 9 and/or 10 of the core. Prodrug moieties may be added to the core as an ester or an amide.

Various embodiments of the invention are described by the following Formula II or a salt thereof:

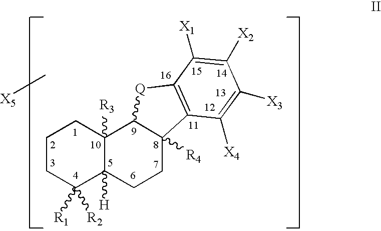

wherein X₁, X₂, X₃, X₄, R₁, R₂, R₃, R₄ and Q are all as defined above for Formula I and may be limited by any or all of the further limitations for these groups as defined above; and
X₅ is a prodrug moiety as described herein, or any combination of any linking moiety as described herein bonded to any solubilizing moiety as described herein and at least one of R₁, R₂, R₃, R₄, X₁, X₂, X₃ and X₄ is/are substituted on, substituted with and/or substituted by X₅ and/or X₅ is a substituent on any carbon atom in Q or in positions 1, 2, 3, 4, 5, 6, 7, 8, 9 and/or 10 of Formula II.

Various embodiments of the invention are described by the following Formula III or a salt thereof:

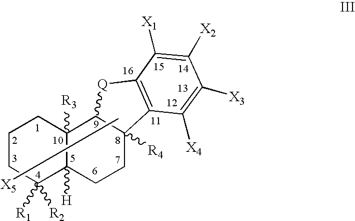

wherein X₁, X₂, X₃, and X₄, are all as defined above for Formula I and may be limited by any or all of the further limitations for these groups as defined above; and
X₅ is a prodrug moiety as described herein and may be a substituent on any methylene or methyl carbon of the terpenoid fragment. In various embodiments, X₅ is joined via an ester linkage (or —O— in the case of phosphates).

Various embodiments of the invention are described by the following Formula IV or a salt thereof:

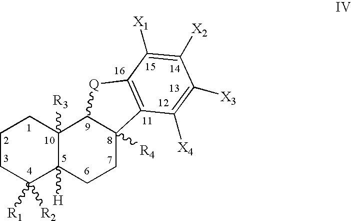

wherein X₁, X₂, X₃, X₄, R₁, R₂, R₃, R₄ and Q are all as defined above for Formula I and may be limited by any or all of the further limitations for these groups as defined above except that one or more of X₁, X₂, X₃ and X₄, is substituted with, substituted in, substituted on, or substituted by X₅; and
X₅ is a prodrug moiety as described herein, or any combination of any linking moiety as described herein bonded to any solubilizing moiety as described herein.

Various embodiments of the invention are described by the following Formula V or a salt thereof:

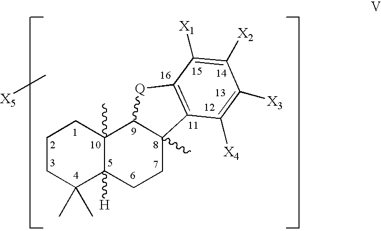

wherein X₁, X₂, X₃, X₄, and Q are all as defined above for Formula I and may be limited by any or all of the further limitations for these groups as defined above except that one or more of X₁, X₂, X₃, and/or X₄, is substituted with, substituted in, substituted on, or substituted by X₅ or may be a substituent on any methylene or methyl carbon of the terpenoid fragment; and
wherein X₅ is a prodrug moiety as described. In various embodiments, X₅ is joined via an ester linkage (or —O— in the case of phosphates).

Various embodiments of the invention are described by the following Formula VI or a salt thereof:

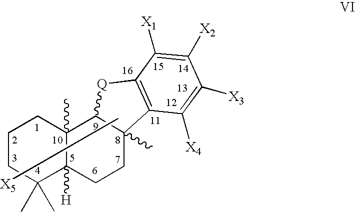

wherein X₁, X₂, X₃, X₄, and Q are all as defined above for Formula I and may be limited by any or all of the further limitations for these groups as defined above; and
X₅ is a prodrug moiety as described herein and may be a substituent on any methylene or methyl carbon of the terpenoid fragment. In various embodiments, X₅ is joined via an ester linkage (or —O— in the case of phosphates).

Various embodiments of the invention are described by the following Formula VII or a salt thereof:

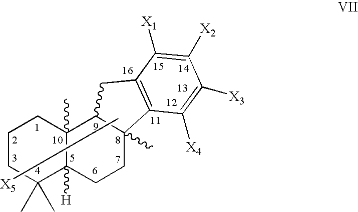

wherein X₁, X₂, X₃, and X₄, are all as defined above for Formula I and may be limited by any or all of the further limitations for these groups as defined above except that one or more of X₁, X₂, X₃, and/or X₄, is replaced with a prodrug moiety joined directly to the aromatic ring via an ester linkage (or an —O— in the case of phosphates) or at least one of the prodrug moieties is attached, as a substituent, via an ester linkage (or an —O— in the case of phosphates) to at least one of X₁, X₂, X₃ and/or X₄.

Various embodiments of the invention are described by the following Formula VIII or a salt thereof:

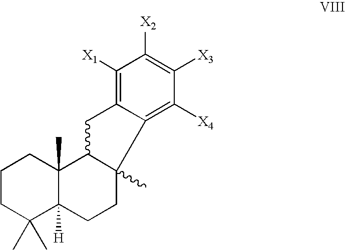

wherein X₁ is selected from the group consisting of: a prodrug moiety, O-methyl and H; X₂ is selected from the group consisting of: a prodrug moiety, O-methyl and H; X₃ is H or methyl; and X₄ is selected from the group consisting of: H, methyl and O-methyl.

In other embodiments of Formula VIII, X₁ is selected from the group consisting of: a prodrug moiety, O-methyl and H; X₂ is selected from the group consisting of: a prodrug moiety, O-methyl and H; X₃ is H or methyl; X₄ is selected from the group consisting of: H, methyl and O-methyl; and exactly one of X₁ and X₂ is a prodrug moiety.

In other embodiments of Formula VIII, X₁ is a prodrug moiety and X₂ is a prodrug moiety. X1 and X2 may have the same prodrug moiety or a different prodrug moiety.

In other embodiments of Formula VIII, X₁ is H and X₂ is a prodrug moiety.

In other embodiments of Formula VIII, X₃ is methyl.

In other embodiments of Formula VIII, X₄ is H.

In other embodiments of Formula VIII, X₁ is H, X₂ is a prodrug moiety, X₃ is H; and X₄ is methyl.

Shown below in Table V are non-limiting examples of the stereoisomers that are specifically encompassed by any one of Formulas I to VIII as depicted above. Stereo-mixtures and racemic mixtures of any two or more of the stereoisomers of Table V, substantially stereo-pure compounds and stereo-pure compounds are also included by Formulas I to VIII as depicted above. X₁, X₂, X₃, X₄, R₁, R₂, R₃, and R₄ as used below in Table V are as defined for the respective Formula. Q as used below in Table V may be present or CH₂, or as defined by any of Formulas I to VIII.

Table V

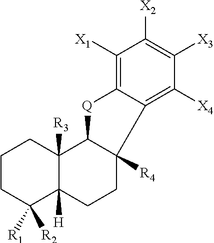

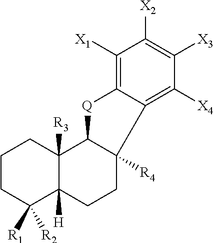

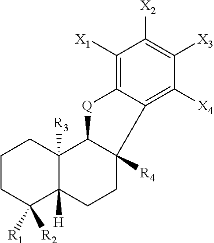

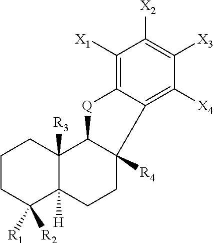

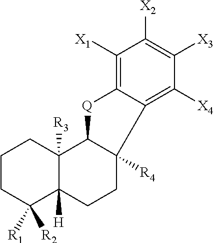

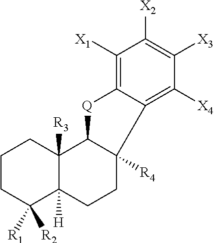

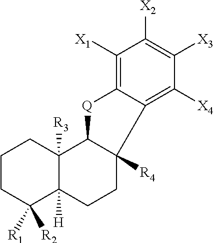

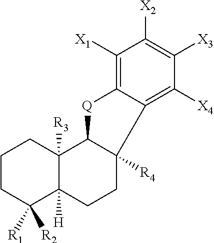

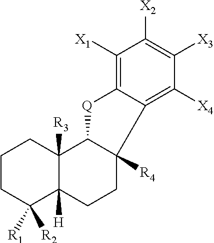

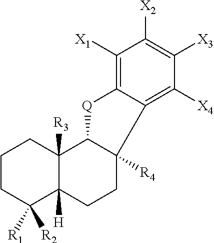

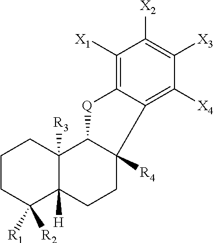

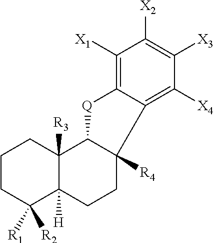

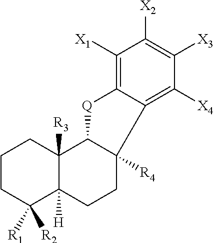

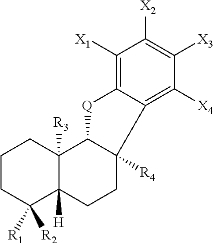

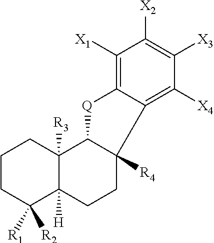

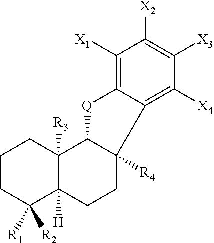

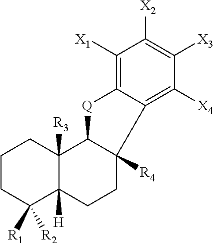

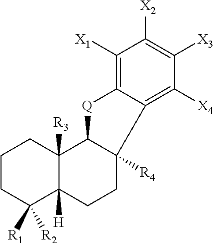

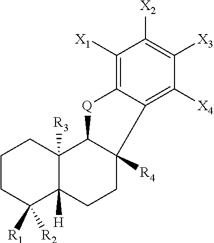

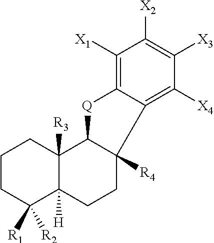

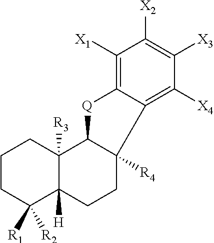

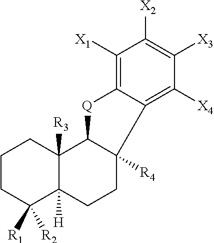

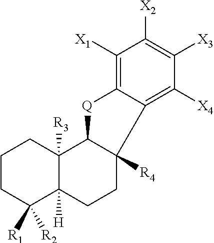

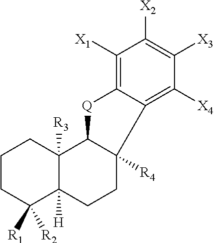

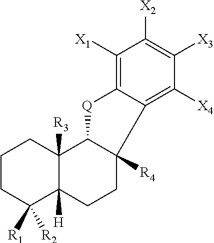

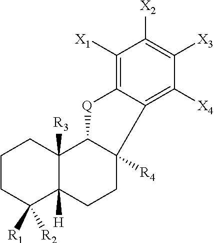

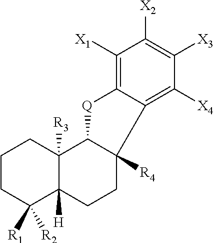

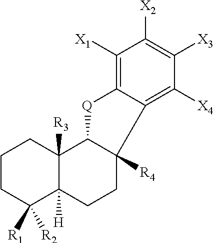

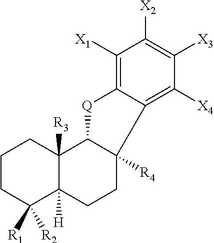

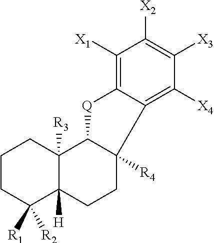

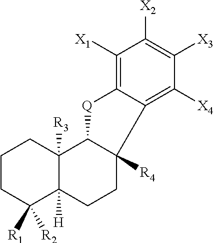

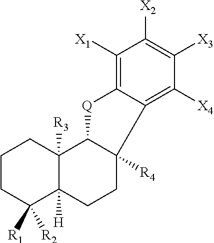

In various particular embodiments, in each of Formulas II, III, IV, V, VI, VII and VIII, the prodrug moiety or X₅ is one of the ester prodrug moieties as set out in Table IV above.

The structures in following Table VI are non-limiting examples of embodiments of this invention. Although relative stereochemistry may or may not illustrated for each structure, the configuration of chiral centres may vary according to any of the embodiments based on chirality described above and compounds of this invention include all stereoisomers and enantiomers of compounds of Formulas II, III, IV, V, VI, VII and/or VIII. In some embodiments, stereo-pure compounds, substantially stereo-pure compounds, stereo-mixtures and racemic mixtures of the compounds of Table VI are also provided. In the following structures X₅ is as described above in any one of Formulas II, III, IV, V, VI, VII or VIII. In Table VI, X₁, X₃, and X₄ are as defined above for Formula I and may be limited by any or all of the further limitations for these groups as defined above.

TABLE VI
NON-LIMITING EXAMPLES OF SHIP MODULATING PRODRUGS

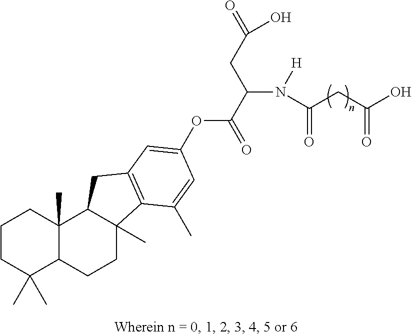

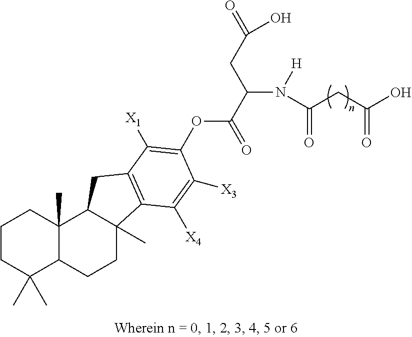

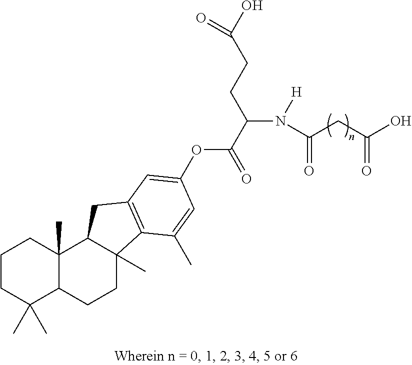

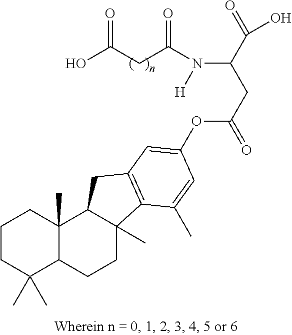

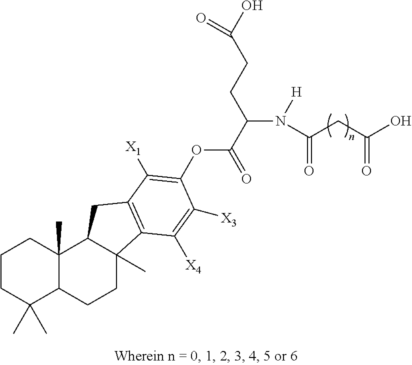

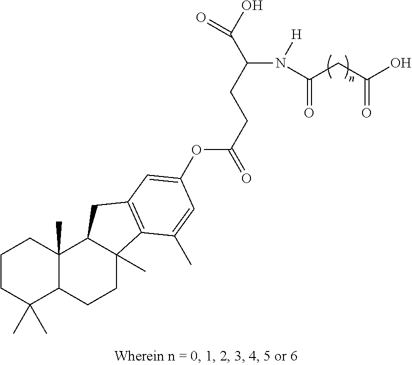

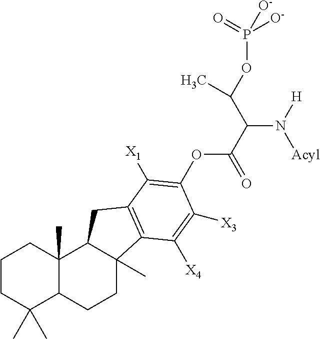

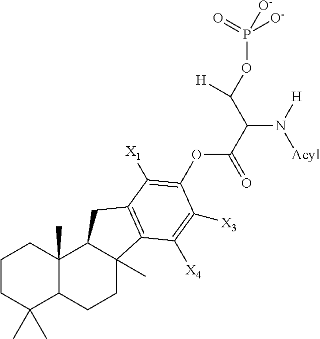

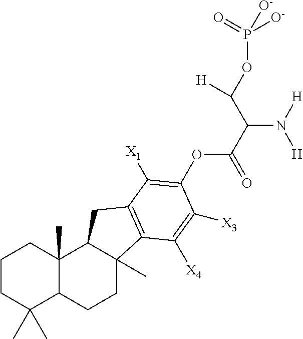

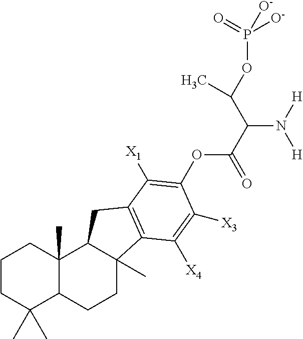

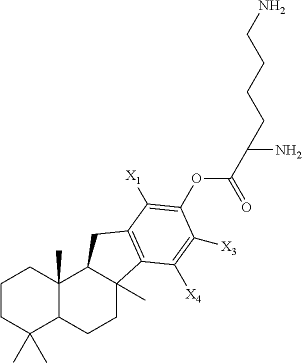

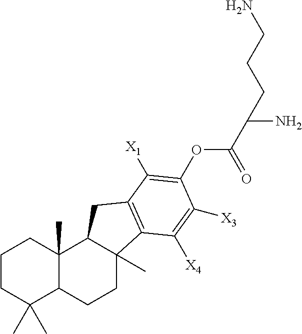

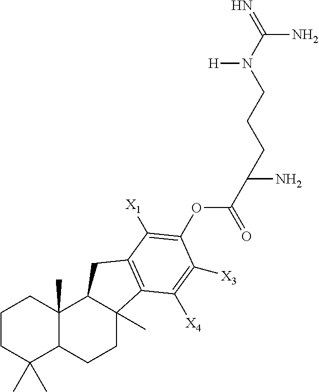

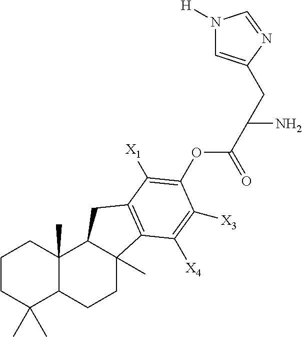

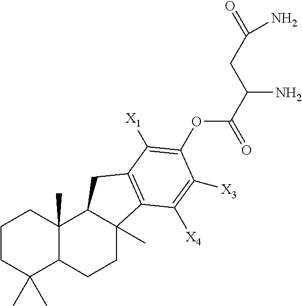

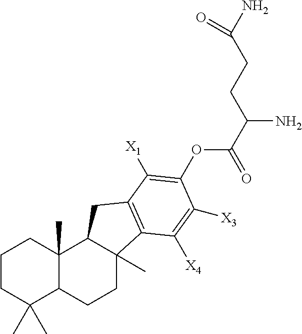

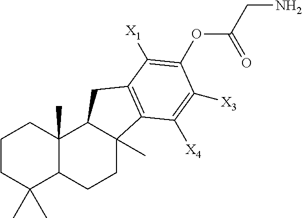

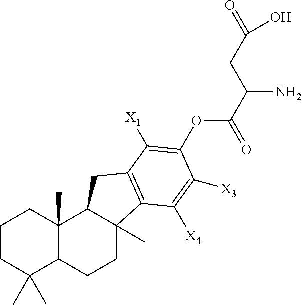

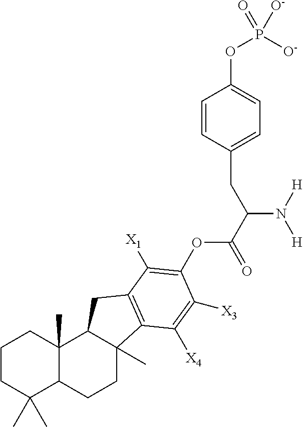

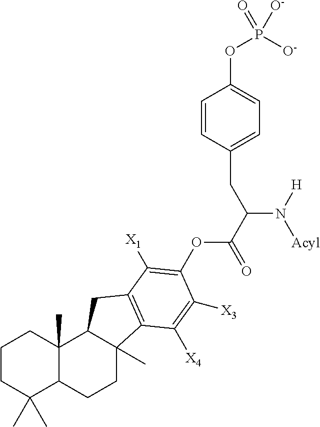

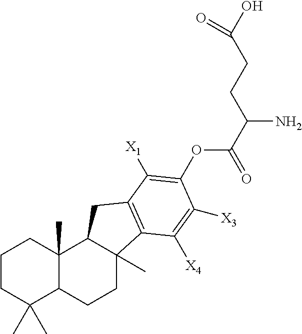

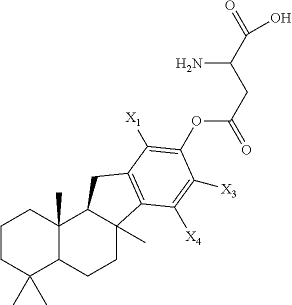

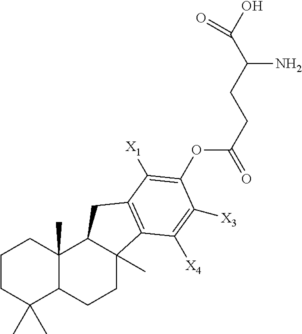

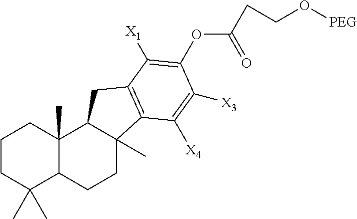

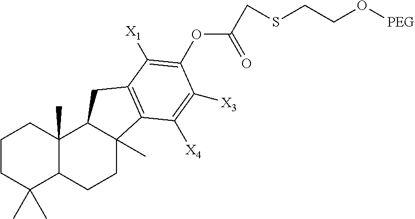

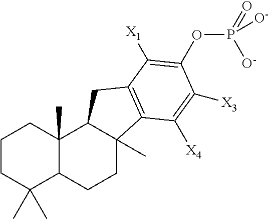

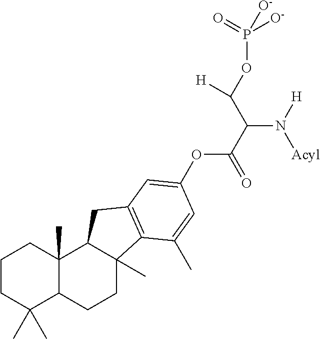

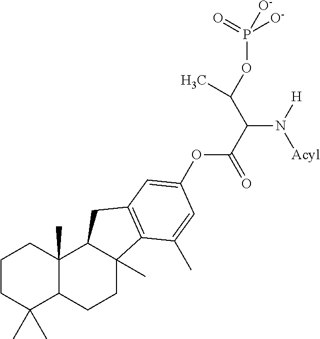

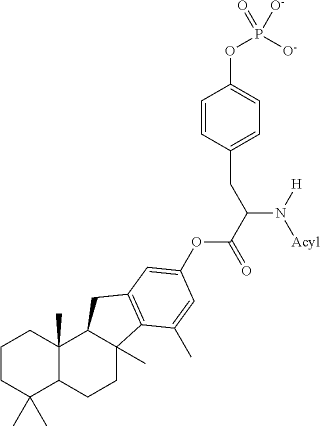

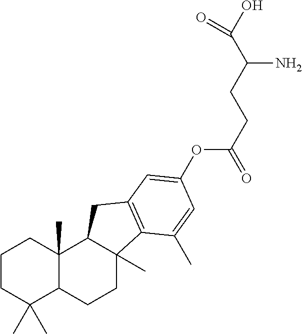

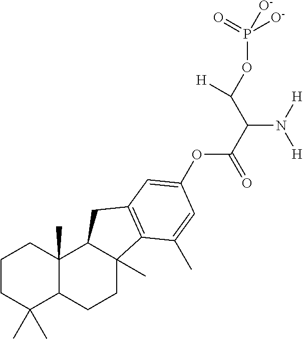

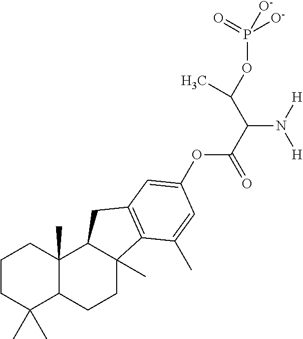

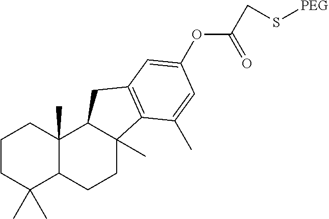

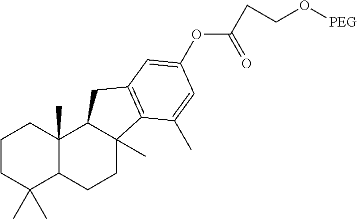

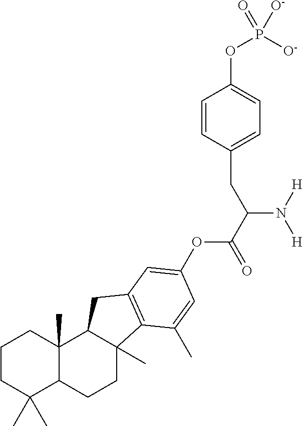

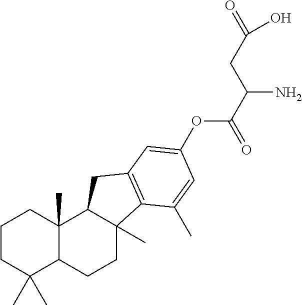

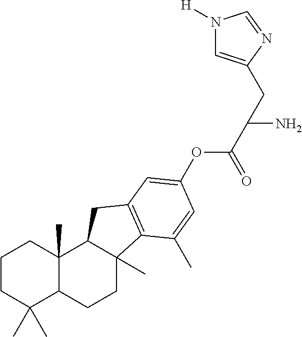

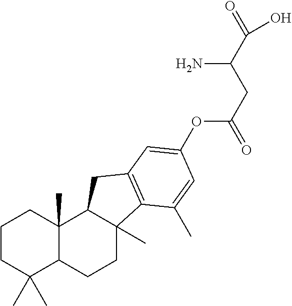

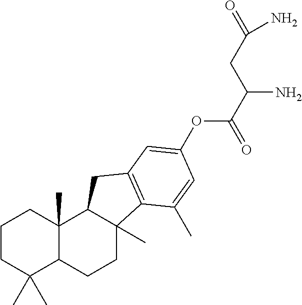

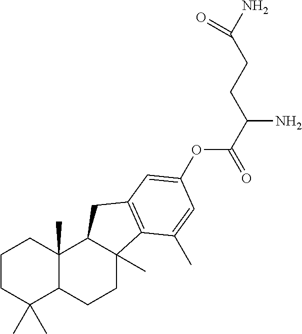

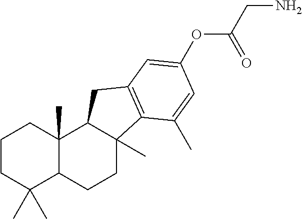

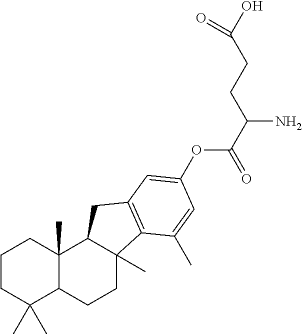

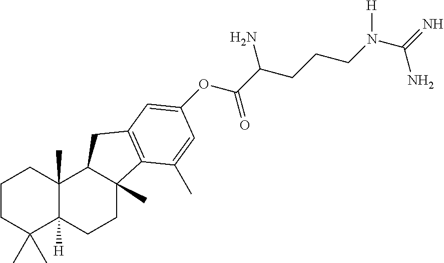

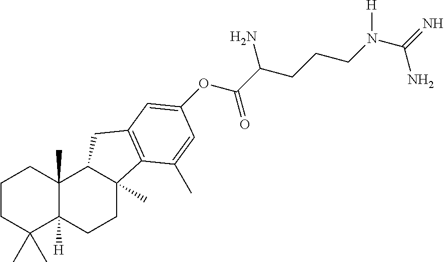

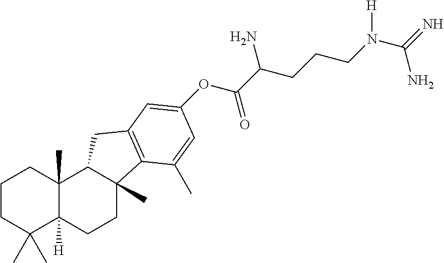

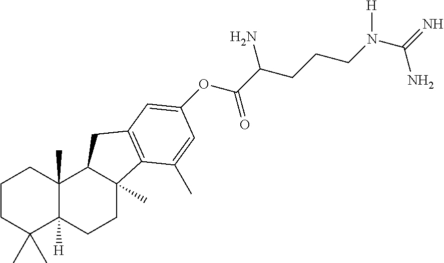

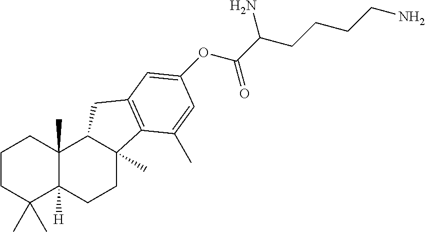

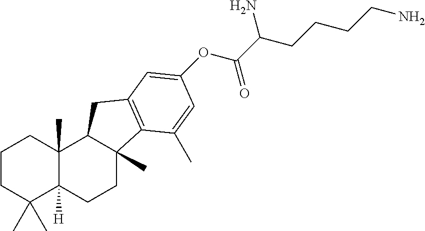

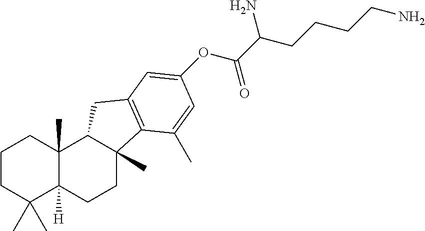

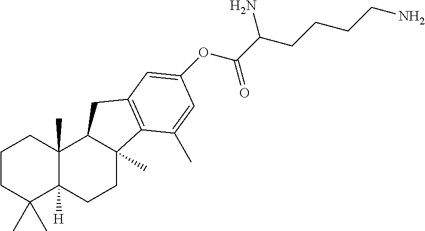

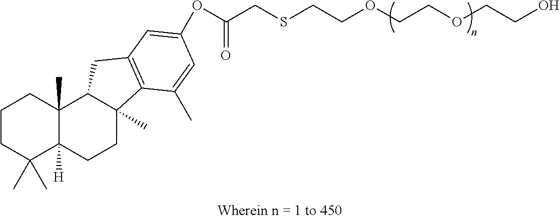

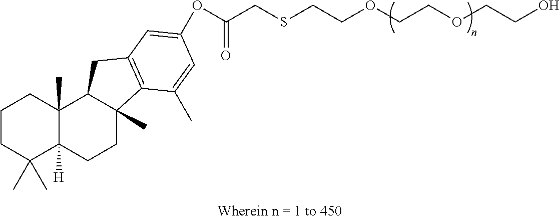

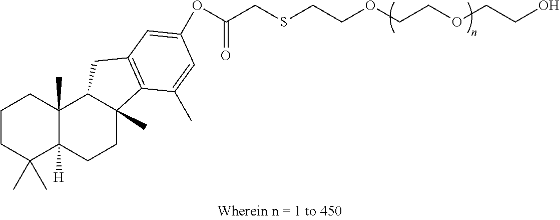

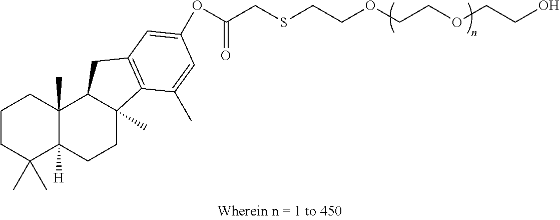

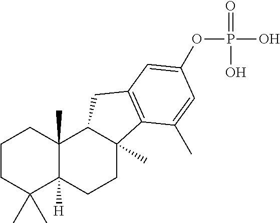

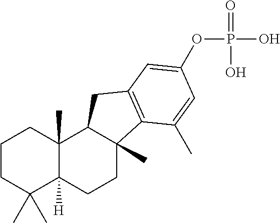

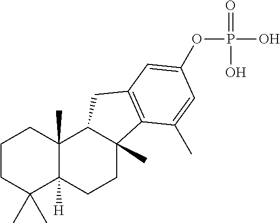

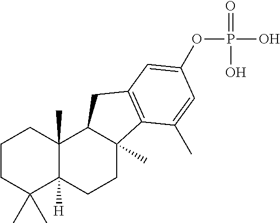

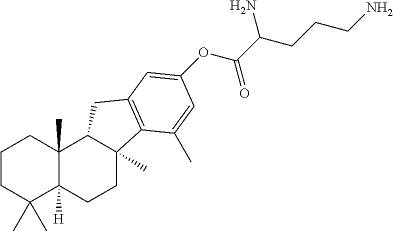

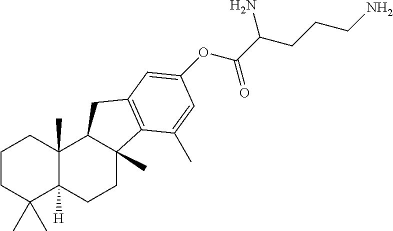

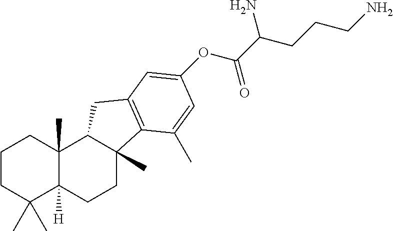

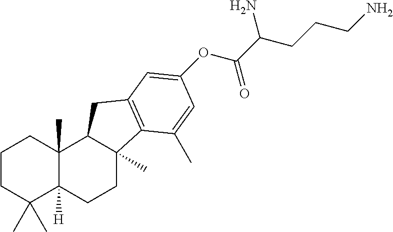

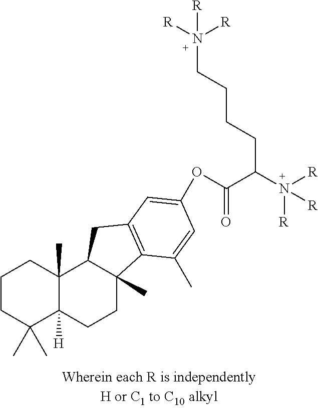

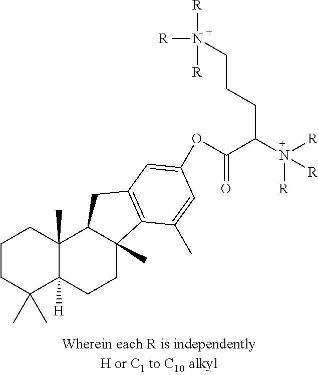

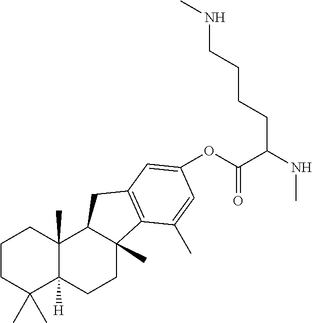

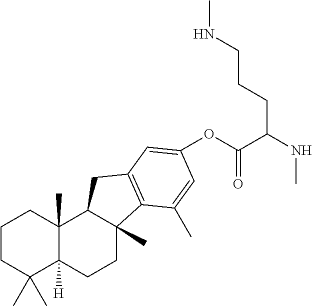

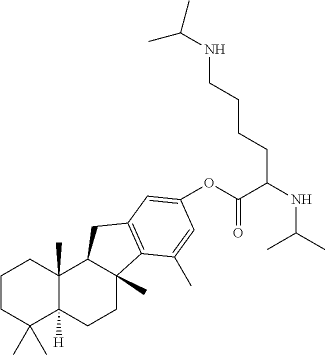

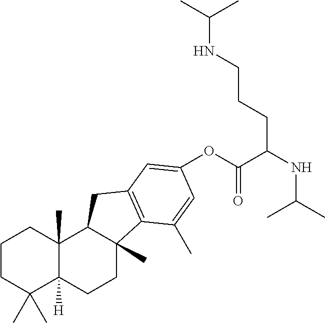

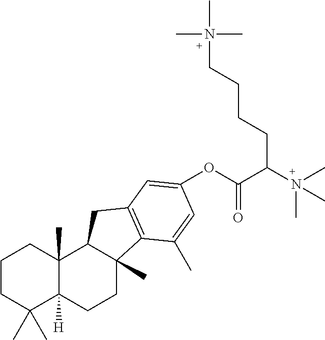

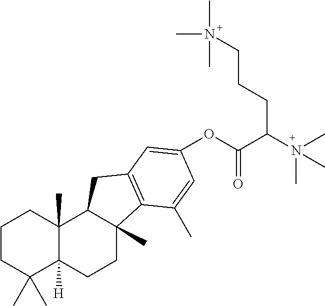

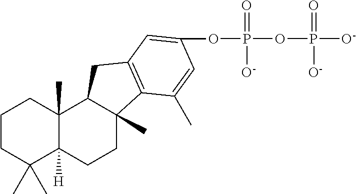

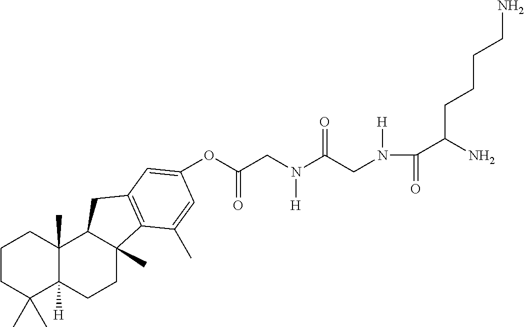

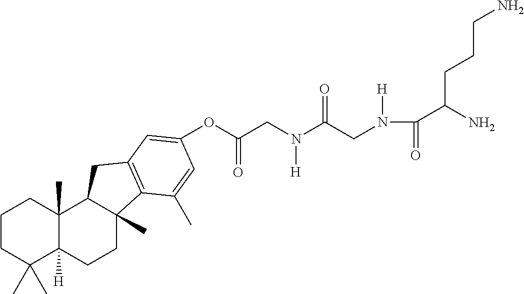

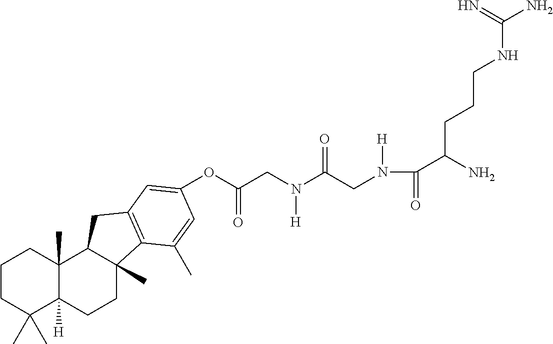

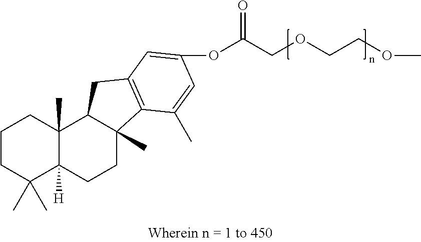

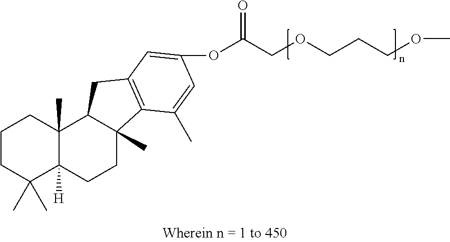

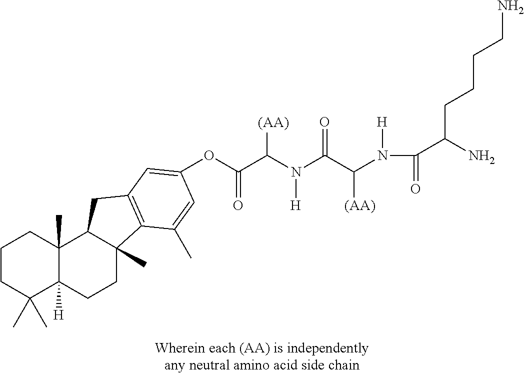

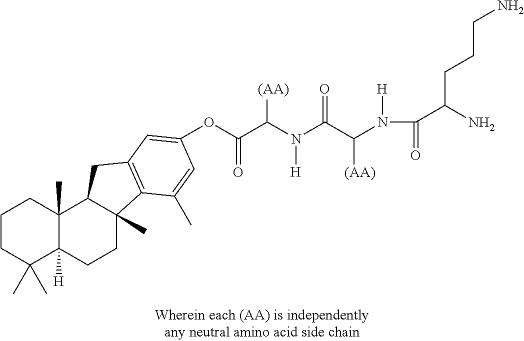

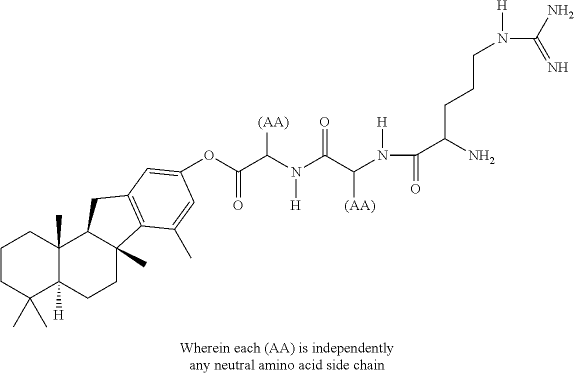

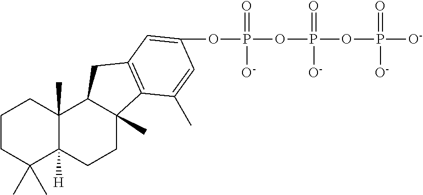

Compounds of the invention are often made by preparing or purchasing a core, preparing or purchasing a prodrug moiety and chemically joining the two moieties. Other methodologies may also be used, including chemically joining a prodrug moiety to a portion of a core and chemically joining the remainder of the core. Details regarding the synthesis of compounds of the invention are described below.

### Synthesis of Compounds and Assays for Activity

Pelorol may be obtained from natural sources as taught in the prior art. Solvent fractionation and/or chromatography may be employed. It is possible to modify pelorol or other available compounds such as chrysene derivatives by known chemical methodologies to add, remove, or replace substituents in order to produce compounds of Formulas II, III, IV, V, VI, VII and/or VIII. Examples of such derivatization steps as applied to different compounds of Formulas II, III, IV, V, VI, VII and/or VIII are shown in more detail below.

The presence of SHIP 1 modulating compounds in a preparation may be determined by use of a variety of assays, including by biological assays which may be readily adapted from known procedures, including cell or animal based assays which monitor changes in: nitric oxide production from activated macrophages; IgE induced mast cell degranulation; LPS induced macrophage activation; TNF-α expression or activity. In addition, standard assays for agents which mediate inflammatory activity in living subjects may be employed. Adaptation of these assays is facilitated by the availability of SHIP 1^{-/-} and SHIP 1^{+/-} mice and bone marrow derived macrophages. In addition, the availability of anti-SHIP 1 antibodies facilitates use of immunoassay formats. Such assays may also be used to assess activity of compounds prepared by total synthesis, as described herein.

### Total Synthesis of Compounds

A synthetic scheme for making embodiments of the invention and intermediates and precursors of embodiments of the invention is provided herein. Tables (VII, VIII and IX) provide examples of embodiments, intermediates and precursors of embodiments of such a synthesis with examples of different compounds of the invention which may be prepared. The compound shown in the Tables that is identical to pelorol except that the ring adjacent the aromatic ring has six members, is termed “homopelorol”. Compounds having a six-membered ring are termed “homopelorol analogs”. Compounds having a five-membered ring other than pelorol are termed herein, “pelorol analogs”.

In the synthesis methods shown in Tables VII, VIII and IX, compounds of the invention and intermediates of compounds of the invention shown therein may be conveniently based on sclareolide as a starting material. Appropriate derivatives of sclareolide providing desired R₁, R₂, R₃ and/or R₄ substituents may be employed. Nu is a nucleophile, often a lithium salt of a carbanion or lithium, and X₂ is often an activating group such as —OMe or —NHAc in the aromatic compound shown in Tables VII and VIII. Gₙ, Gₓ, G_{y} and Gz in Table VII are as defined herein for X₁, X₂, X₃ and X₄, respectively and in all of Tables VII to IX, X₁, X₂, X₃, X₄, R₁, R₂, R₃, R₄ and/or Q may remain as found in the starting material or be appropriately altered to provide the desired substituents for the end product. Protecting groups may be employed on R₁, R₂, R₃ and/or R₄ or X₁, X₃, or X₄.

In the synthesis methods shown in Table IX, the starting compound may conveniently be a core, the synthesis of which are described in the prior art (see for example international publication number WO 2004/035601, which is incorporated herein by reference).

A general procedure for preparation of phosphate ester compounds may be found in Steinber, G. M. J. Org. Chem. (1950), 15, 637. A specific procedure for tyrosine phosphorylation may be found in Gibson, B. W. et. al. J. Am. Chem. Soc. (1987), 109, 5343. A general description of pegylating compounds may be found in Zhu et al., J. Med. Chem. (2006), 49 1373-1378. More specific and detailed examples of syntheses of compounds of the invention may be found in the examples.

TABLE VII
Synthesis of Pelorol and Pelorol Analog Core

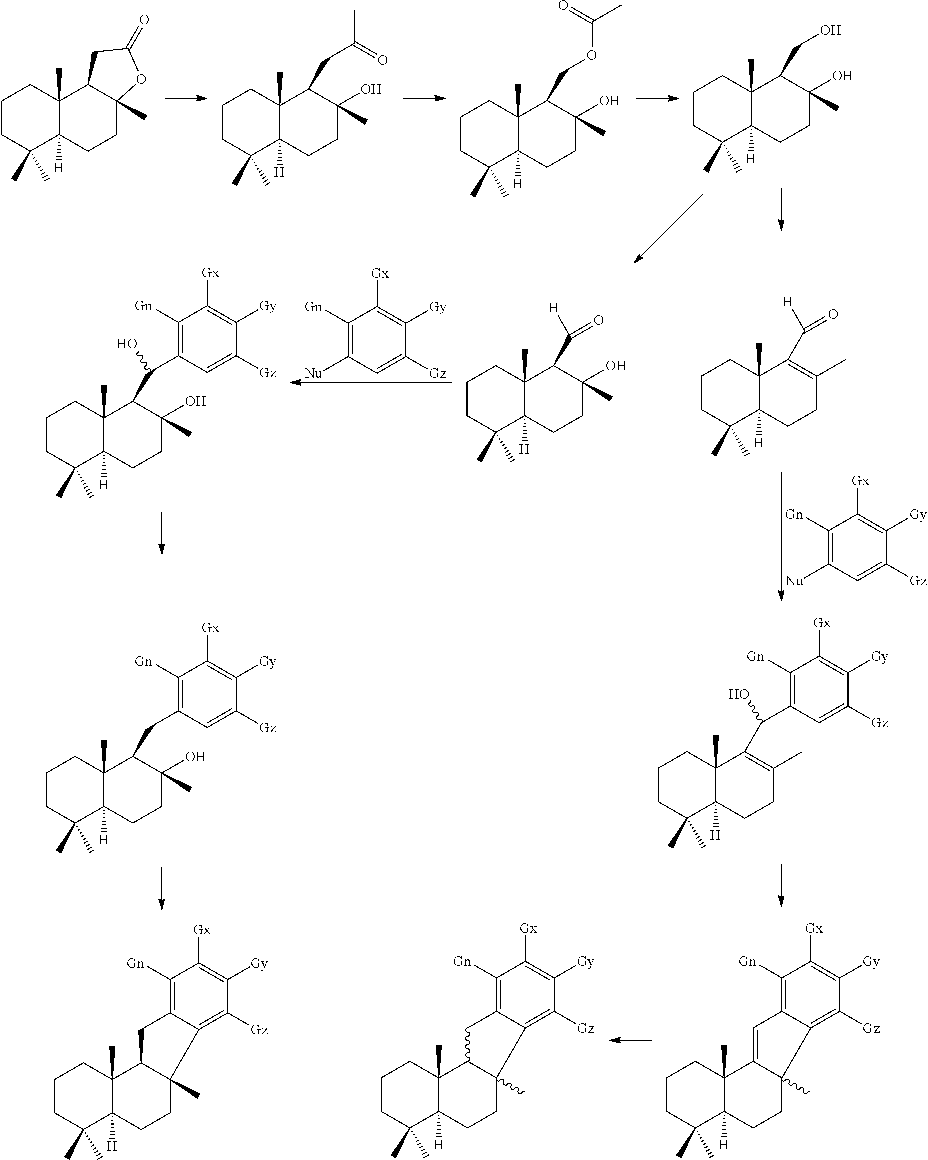

TABLE VIII
Synthesis of Homopelorol and Homopelorol Analog Core

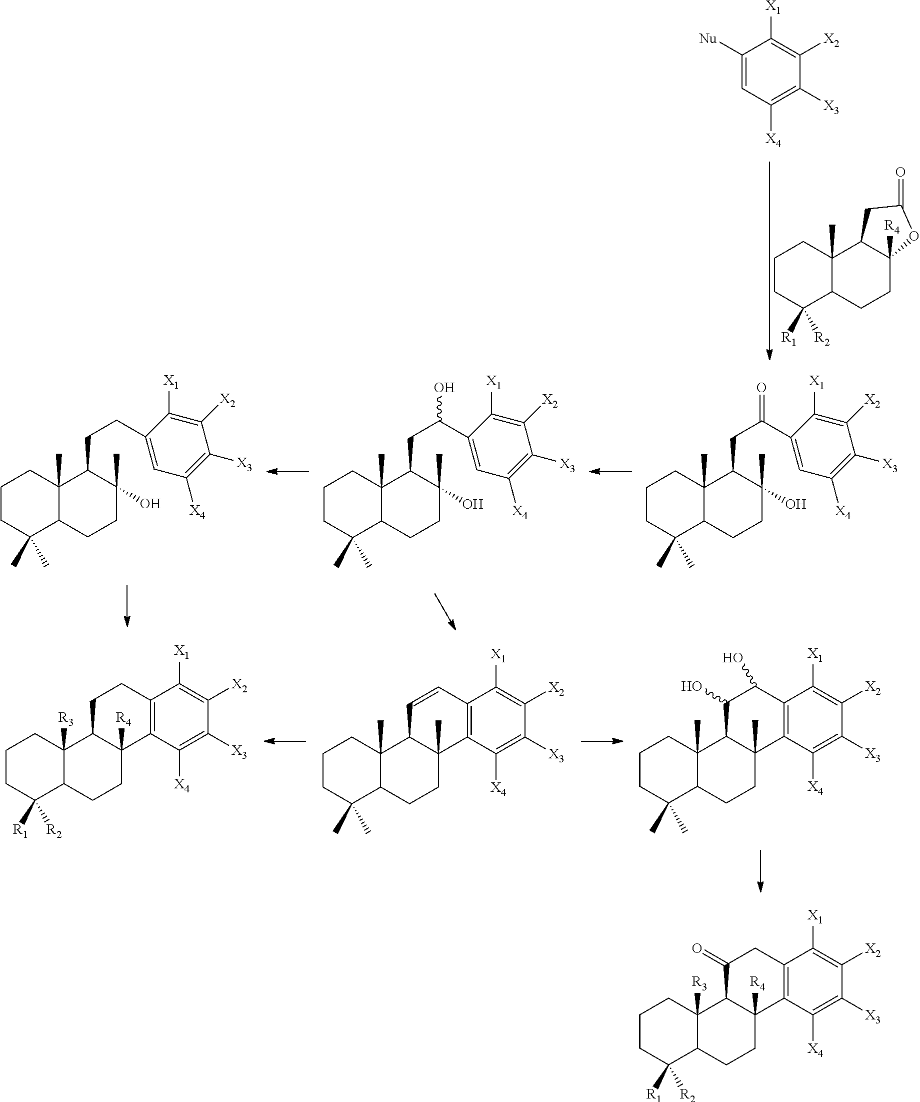

TABLE IX
General Synthesis of Some Prodrugs
X₂ is used as a non-limiting example of the target site for addition of the prodrug moiety.

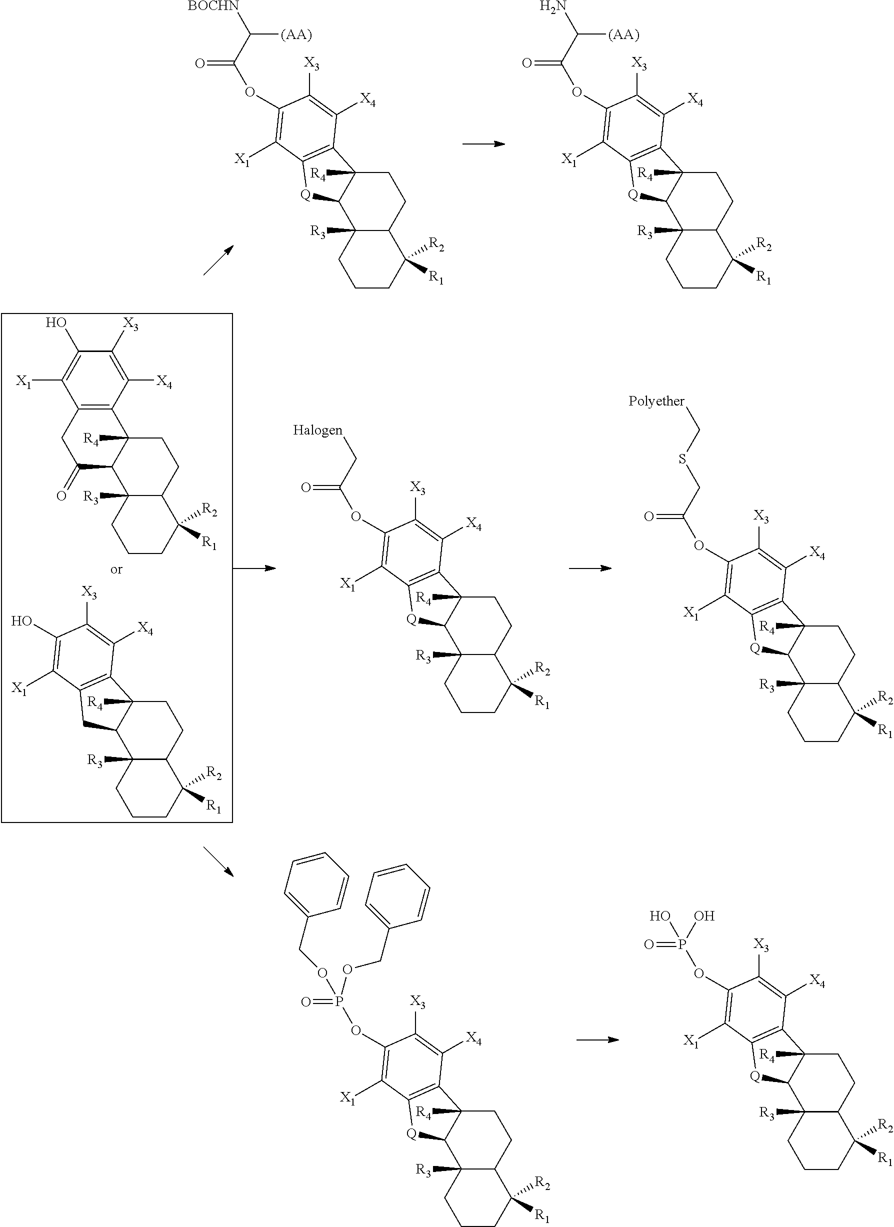

### Pharmaceutical Compositions, Dosages, Administration and Indications

Compounds for use in this invention may be formulated into pharmaceutical compositions in any number of ways, which would be known to a person of skill in the art, all of which are within the scope of the invention. The person of skill in the art may be expected to select appropriate pharmaceutically acceptable salts as well as appropriate pharmaceutically acceptable excipients, diluents, and carriers.

Compounds according to the invention can be provided in therapeutically- or prophylactically-acceptable amounts, in any pharmaceutically acceptable carrier. Methods well known in the art for making such pharmaceutical formulations are found in, for example, “Remington: The Science and Practice of Pharmacy” (21^{st} edition), ed. A. Gennaro, 2005, Mack Publishing Company, Easton, Pa., incorporated by reference herein. Pharmaceutical formulations according to the present invention may, for example, contain excipients, sterile water, or saline, ethanol, methanol, dimethyl sulfoxide, polyalkylene glycols such as polyethylene glycol, propylene glycol, or other synthetic solvents, oils of vegetable origin, or hydrogenated naphthalenes.

Compounds according to the invention may include hydrophobic compounds, for example, compounds that are substantially insoluble in water, but are freely soluble in solvents such as, for example, ethanol, methanol, dimethyl sulfoxide, or chloroform, or combinations thereof. Formulations containing such hydrophobic compounds may be provided using, for example, micelles, which are formed by amphiphilic compounds under certain conditions. In aqueous solutions, micelles are capable of incorporating hydrophobic compounds in their hydrocarbon cores, or within the micelle walls. Hydrophobic compounds may also be provided by solubilization in triglycerides (oils), for example, a digestible vegetable oil. The solubilized hydrophobic compound in the oil phase may be dispersed in an aqueous solution and stabilized using emulsifying agents, if desired. Alternatively, the hydrophobic compound may be provided in oil and delivered, for example, to the gastrointestinal system where bile salts may function as in vivo emulsifiers. Hydrophobic compounds may also be provided as microemulsions which, like emulsions, are liquid dispersions of oil and water, but have smaller particles with an oil phase in a micelle-like “core.” Hydrophobic compounds according to the invention may also be provided together with a polymeric carrier, for example, a carbohydrate such as starch, cellulose, dextran, cyclodextrin, methylcellulose, or hyaluronic acid, or a polypeptide, such as albumin, collagen, or gelatin. Other modes of formulation of hydrophobic compounds may include liposomes, natural and synthetic phospholipids, or solvents, for example, dimethyl sulfoxide or alcohols.

The pharmaceutical compositions of the invention may be formulated so as to provide controlled release of the active compound(s) over a period of time. Thus, the formulations could contain, for example, an amount of the compound that would be toxic if administered as a single dose, but whose controlled release does not exceed toxic levels. Biocompatible, biodegradable lactide polymer, lactide/glycolide copolymer, or polyoxyethylene-polyoxypropylene copolymers, for example, may be used to control the release of the compounds. Other potentially useful delivery systems for modulatory compounds according to the present invention include ethylene-vinyl acetate copolymer particles, osmotic pumps, implantable infusion systems, and liposomes.

A “therapeutically effective amount” of a compound is an amount effective, at dosages and for periods of time necessary, to achieve the desired therapeutic result using a compound according to the invention. A therapeutically effective amount is also one in which any toxic or detrimental effects of the compound are outweighed by the therapeutically beneficial effects. A “prophylactically effective amount” of a compound refers to an amount effective, at dosages and for periods of time necessary, to achieve the desired prophylactic result. Typically, a prophylactic dose is used in subjects prior to or at an earlier stage of disease, so that a prophylactically effective amount may be less than a therapeutically effective amount. Amounts considered sufficient will vary according to the specific compound used, the mode of administration, the stage and severity of the disease, the age, sex, weight, and health of the individual being treated, and concurrent treatments.

A preferred range for therapeutically or prophylactically effective amounts of the compounds of the invention may be 0.1 nM-0.1M, 0.1 nM-0.05M, 0.05 nM-15 μM 0.01 nM-10 μM, 0.1 μM-1 μM, 0.1 μM-0.6 μM or 0.3 μM-0.6 μM. It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. Dosage regimens may be adjusted to provide the optimum therapeutic response. For example, a single bolus may be administered, several divided doses may be administered over time or the dose may be proportionally reduced or increased as indicated by the exigencies of the therapeutic situation.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions.

Conventional pharmaceutical practice may be employed to provide suitable formulations or compositions to administer the compounds to patients, depending on the therapeutic or prophylactic objectives. Any appropriate route of administration may be employed, for example, systemic, parenteral, intravenous, subcutaneous, transdermal, transmucosal, intramuscular, intracranial, intraorbital, ophthalmic, intraventricular, intracapsular, intraspinal, intracisternal, intraperitoneal, intranasal, aerosol, topical, surgical, or oral administration. The formulations used may vary according to the chosen route of administration. Thus, for oral administration, the formulations may be in the form of tablets or capsules; for inhalants, the formulations may be in the form of powders, nasal drops, or aerosols; for transmucosal administration, the formulations may be nasal sprays or suppositories; for transdermal administration, the formulations may be creams, ointments, salves, or gels; etc.

Therapeutically effective or prophylactically effective amounts of SHIP 1 modulators and pharmaceutical compositions of this invention may be administered to patients in need of treatment or prophylaxis for cancer (neoplastic diseases), other cell proliferative disorders, inflammatory diseases and immune diseases. Neoplastic diseases include but are not limited to: leukemias, carcinomas, sarcoma, melanomas, neuroblastoma, capillary leak syndrome and hematological malignancies. Diseases with an inflammatory component include, but are not limited to: rheumatoid arthritis, multiple sclerosis, Guillan-Barre syndrome, Crohn's disease, ulcerative colitis, inflammatory bowel syndrome, psoriasis, graft versus host disease, host versus graft, lupus erythematosis, Alzheimer's disease and insulin-dependent diabetes mellitus. Diseases related to inappropriate activation of macrophage-related cells of the reticuloendothelial lineage include osteoporosis.

Pelorol and other compounds having the structure of Formulas I-VIII exhibit SHIP 1 agonist activity. By activating SHIP 1, such agonists are particularly useful in the treatment of inflammatory diseases such as sepsis/septic shock, colitis, inflammatory bowel syndrome, and those involving macrophage proliferation or activation; neoplastic diseases such as myeloid and lymphoid leukemias; as an immunosuppressive agent such as in transplant rejection; hematopoietic disorders; and for affecting mast cell degeneration such as in the treatment or prophylaxis of allergies.

In the following Examples and Figures, the terms “Compound #”, “MN#” “AQXMN#” and “AQX-MN#” are all equivalent. For example, “Compound 100” is the same as “MN100” and is the same as “AQXMN100” and is the same as “AQX-MN100”.

### Example 1

### Synthesis of Compound 100

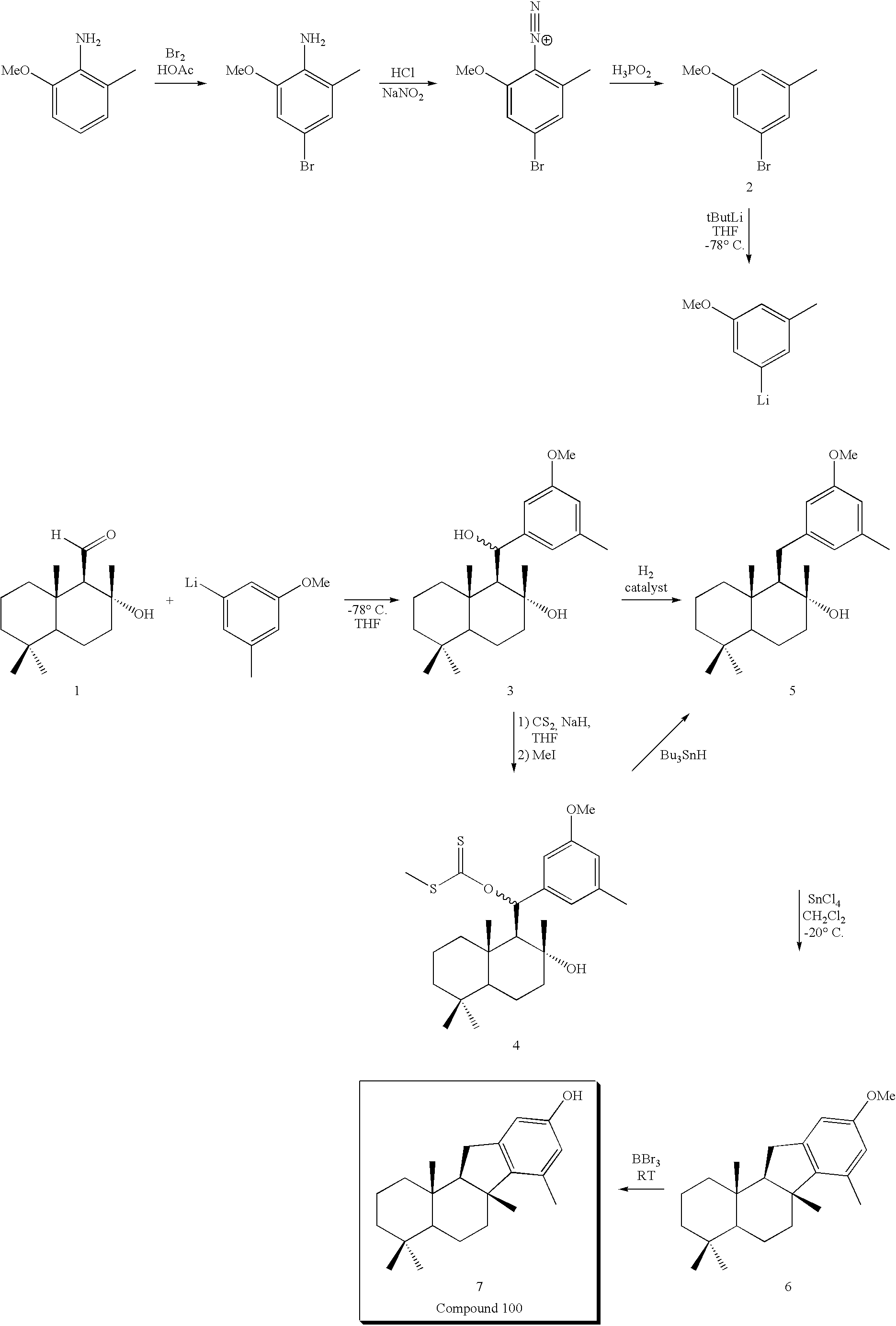

Drimane-8α,11-diol was prepared according to Kuchkova et al; Synthesis, 1997, 1045

Bromomethoxytoluene (2) was prepared according to Chan et al; J. Med. Chem. (2001), 44, 1866

### Preparation of Aldehyde (1)

Drimane-8α,11-diol (17.5 g, 72.8 mmol) was dissolved in 1 L CH₂Cl₂. Diisopropylethylamine (50.7 mL, 291.2 mmol) was added and the solution was cooled to −15° C. A solution of Pyr-SO₃ (46.3 g, 291.2 mmol) in DMSO (250 mL) was added dropwise over a period of 20 min, then the reaction was stirred cold for an additional 5 min. To the cold reaction was added 1M HCl (500 mL), and the organic layer was partitioned. The aqueous layer was washed with an additional 200 mL CH₂Cl₂. The pooled organic layers were then washed with saturated NaHCO₃, dried over Na₂SO₄ and concentrated. The crude product was purified by column chromatography (Hex: EtOAc) to yield 10.5 g aldehyde (1) (44.1 mmol, 60.1% yield) as a white semisolid.

### Preparation of Diol (3)

Bromomethoxytoluene (2) (3.64 g, 18.29 mmol) was dissolved in 35 mL dry THF under an argon atomosphere. This solution was cooled to −78° C., and tBuLi (21.5 mL, 36.6 mmol) was added dropwise via syringe. The solution was stirred for 10 min at −78° C., then warmed to RT for 20 min. The solution was re-cooled to −78° C., and a solution of aldehyde (1) (1.45 g, 6.09 mmol) in 6 mL dry THF was added via syringe. The solution was stirred at −78° C. 2 h, after which the reaction was quenched with the addition of 1M HCl. EtOAc (100 mL) was added, and the organic phase was washed with 1M HCl, followed by saturated NaHCO₃. The organic phase was dried over MgSO₄, filtered and concentrated. The crude reaction mixture was purified by column chromatography (Hex:EtOAc) to yield diol (3) (1.94 g, 88.5% yield).

¹H NMR (CDCl₃) δ 0.34 (td, J=13.3, 3.6 Hz, 1H), 0.77 (s, 3H), 0.82 (s, 3H), 0.90 (m, 1H), 0.97 (td, 13.5, 3.6 Hz, 1H), 1.02 (s, 3H), 1.13 (m, 1H). 1.16 (m, 1H), 1.23 (m, 1H) 1.33 (m, 1H), 1.40 (m, 1H), 1.54 (s, 3H), 1.56 (m, 1H), 1.63 (m, 1H), 1.84 (dt, 12.2, 3.3 Hz, 1H), 2.12 (d, 8.1 Hz, 1H), 2.33 (s, 3H), 3.79 (s, 3H), 4.79 (d, 8.1 Hz, 1H), 6.61 (s, 1H), 6.78 (s, 1H), 6.85 (s, 1H).

¹³C NMR (CDCl₃) δ 15.9, 18.3, 19.8, 21.50, 21.53, 26.1, 33.2, 33.5, 38.6, 40.8, 41.3, 44.0, 55.1, 55.8, 62.84, 62.85, 76.0, 110.5, 113.6, 120.7, 139.8, 149.0, 159.7

HRESIMS calcd for C₃₂H₃₆O₃Na 383.2562, found 383.2563

### Preparation of Xanthate (4)

Diol (3) (1.94 g, 5.39 mmol) was dissolved in 20 mL dry THF under an argon atmosphere. To this solution was added NaH (237 mg, 60% in oil, 5.93 mmol). The reaction was then heated to 50° C. until the solution was clear orange. The reaction was cooled to 0° C., and CS₂ (1 mL, 16.6 mmol) was added. The solution was stirred for 20 min at 0° C., then warmed to RT for an additional 20 minutes, after which MeI (1 mL, 16.6 mmol) was added. The reaction was stirred at RT for 1 hour, then concentrated to dryness. The crude mixture was dissolved in EtOAc, and washed with 3×H₂O. The organic solution was dried over MgSO₄, filtered and concentrated to yield a mixture of xanthate (4) and fragmentation product, ketone (approx 4:1). This product mixture was used in the next step without further purification.

¹H NMR (CDCl₃) δ 0.56 (td, 12.9, 3.5 Hz, 1H), 0.77 (s, 3H), 0.80 (s, 3H), 0.87 (dd, 12.2, 2.4 Hz, 1H), 0.99 (dt 13.6, 3.8 Hz, 1H), 1.02 (s, 3H), 1.28 (m, 1H), 1.31 (m, 1H), 1.34 (m, 1H), 1.45 (m, 1H), 1.50 (s, 3H), 1.55 (m, 1H), 1.65 (m, 1H), 1.75 (m, 1H), 1.78 (m, 1H), 1.81 (m, 1H), 2.18 (d, 5.2 Hz 1H), 2.28 (s, 3H), 2.38 (s, 3H), 3.75 (s, 3H), 5.18 (d, 5.2 Hz, 1H), 6.5 (s, 1H), 6.7 (s, 1H), 6.8 (s, 1H)

¹³C NMR (CDCl₃) δ 13.0, 15.9, 18.3, 20.2, 21.3, 21.6, 26.3, 33.26, 33.30, 40.2, 41.0, 41.3, 46.0, 46.8, 55.0, 55.9, 65.1, 74.2, 110.9, 112.3, 120.9, 139.5, 149.9, 159.4, 189.7

HRESIMS calcd for C₂₅H₃₈O₃S₂Na 473.2160, found 473.2159

### Preparation of Alcohol (5)

Xanthate (4) and ketone were dissolved as a crude mixture in 50 mL toluene, and placed under an argon atmosphere. Bu₃SnH (2.9 mL, 10.78 mmol) was added, and the solution was heated. Once at reflux, a catalytic amount of VAZO (1,1′-Azobis(cyclohexanecarbonitrile)) (approx 50 mg) was added through the top of the condensor. The solution was refluxed for 1 hour, then an additional amount of VAZO was added (approx 50 mg). The solution was refluxed for another 45 min, after which TLC analysis (20% EtOAc:Hex) indicated the reaction to be complete. The reaction was cooled, then concentrated to dryness. Flash chromatography of the crude product yielded alcohol (5) (1.12 g, 3.23 mmol, 60% yield, 2 steps) as a white foam.

¹H NMR (CDCl₃) δ 0.78 (s, 3H), 0.85 (s, 3H), 0.87 (s, 3H), 0.90 (m, 1H), 0.93 (m, 1H), 0.96 (m, 1H), 1.09 (td, 13.3, 3.9 Hz, 1H), 1.25 (s, 3H), 1.31 (m, 1H), 1.35 (m, 1H), 1.39 (m, 1H), 1.43 (m, 1H), 1.54 (m, 1H), 1.64 (m, 1H), 1.70 (m, 1H), 1.84 (dt, 12.4, 3.1 Hz, 1H), 2.27 (s, 3H), 2.60 (dd, 14.7, 4.5 Hz, 1H), 2.70 (dd, 14.7, 5.9 Hz, 1H), 3.75 (s, 3H), 6.49 (s, 1H), 6.63 (s, 1H), 6.68 (s, 1H)

¹³C NMR (CDCl₃) δ 15.4, 18.4, 20.2, 21.4, 21.5, 24.5, 31.2, 33.2, 33.3, 39.1, 40.3, 41.7, 44.0, 55.0, 56.0, 63.0, 74.1, 111.3, 111.9, 122.1, 139.2, 145.9, 159.5

HRESIMS calcd for C₂₃H₃₆O₂Na 367.2613, found 367.2615

### Preparation of Tetracycle (6)

Alcohol (5) (1.12 g, 3.23 mmol) was dissolved in 10 mL CH₂Cl₂ and cooled to 0° C. To this solution was added SnCl₄ (1 mL) neat. The orange solution was then stirred for 1 hour at 0° C., followed by quenching with MeOH. The reaction was extracted into EtOAc, and washed with 2×satd NaHCO₃. The organic phase was dried over MgSO₄, filtered and concentrated to yield tetracycle (6) (1.05 g, 3.20 mmol, 99% yield). This compound was used without further purification.

¹H NMR (CDCl₃) [symbol] 0.86 (s, 6H), 0.98 (m, 1H), 1.02 (s, 3H), 1.06 (s, 3H), 1.17 (td, 13.5, 4.2 Hz, 1H), 1.24 (m, 1H), 1.40 (m, 2H), 1.54 (m, 2H), 1.71 (m, 4H), 2.27 (s, 3H), 2.34 (m, 1H), 2.49 (dd, 14.5, 6.2 Hz, 1H), 2.60 (m, 1H), 3.74 (s, 3H), 6.41 (s, 1H), 6.62 (s, 1H)

¹³C NMR (CDCl₃) δ 15.7, 17.9, 18.6, 19.1, 19.9, 20.7, 28.6, 32.6, 32.9, 36.5, 37.9, 38.5, 39.7, 42.1, 54.8, 56.7, 64.2, 107.9, 113.4, 117.9, 132.5, 143.8, 157.3

HRESIMS calcd for C₂₃H₃₅O [M+H]⁺ 327.2688, found 327.2685

### Preparation of Compound 100 (7)

Tetracycle (6) (1.05 g, 3.20 mmol) was dissolved in 15 mL DCM. To this solution was added a solution of BBr₃ (1.0M in DCM) (3.20 mL, 3.20 mmol). The solution was stirred at RT for 2 hours, then concentrated to dryness. The brown residue was dissolved in EtOAc, then washed with H₂O until the pH of the aqueous layer was neutral. The crude product was purified by flash chromatography to yield Compound 100 (7) (931 mg, 2.98 mmol, 93% yield) as a white solid.

¹H NMR (CDCl₃) [symbol] 0.88 (s, 6H), 0.97 (m, 1H), 1.00 (m, 1H), 1.04 (s, 3H), 1.07 (s, 3H), 1.18 (td, 13.2, 4.2 Hz, 1H), 1.42 (m, 1H), 1.43 (m, 1H), 1.53 (m, 1H), 1.58 (m, 1H), 1.71 (m, 1H), 1.73 (m, 1H), 1.74 (m, 1H), 1.75 (m, 1H), 2.26 (s, 3H), 2.35 (dt, 11.7, 3.0 Hz, 1H), 2.48 (dd, 14.35, 6.44 Hz, 1H), 2.59 (m, 1H), 6.36 (d, 1.9 Hz, 1H), 6.55 (d, 1.9 Hz, 1H)

¹³C NMR (CDCl₃) δ 16.1, 18.3, 18.8, 19.6, 20.4, 21.1, 29.0, 33.1, 33.4, 37.0, 39.0, 40.1, 42.6, 47.1, 57.1, 64.5, 109.9, 115.1, 133.1, 144.2, 144.7, 153.5

HRESIMS calcd for C₂₂H₃₃O [M+H]⁺ 313.2531, found 313.2526

### Example 2

### Synthesis of Compound 124 and Compound 125

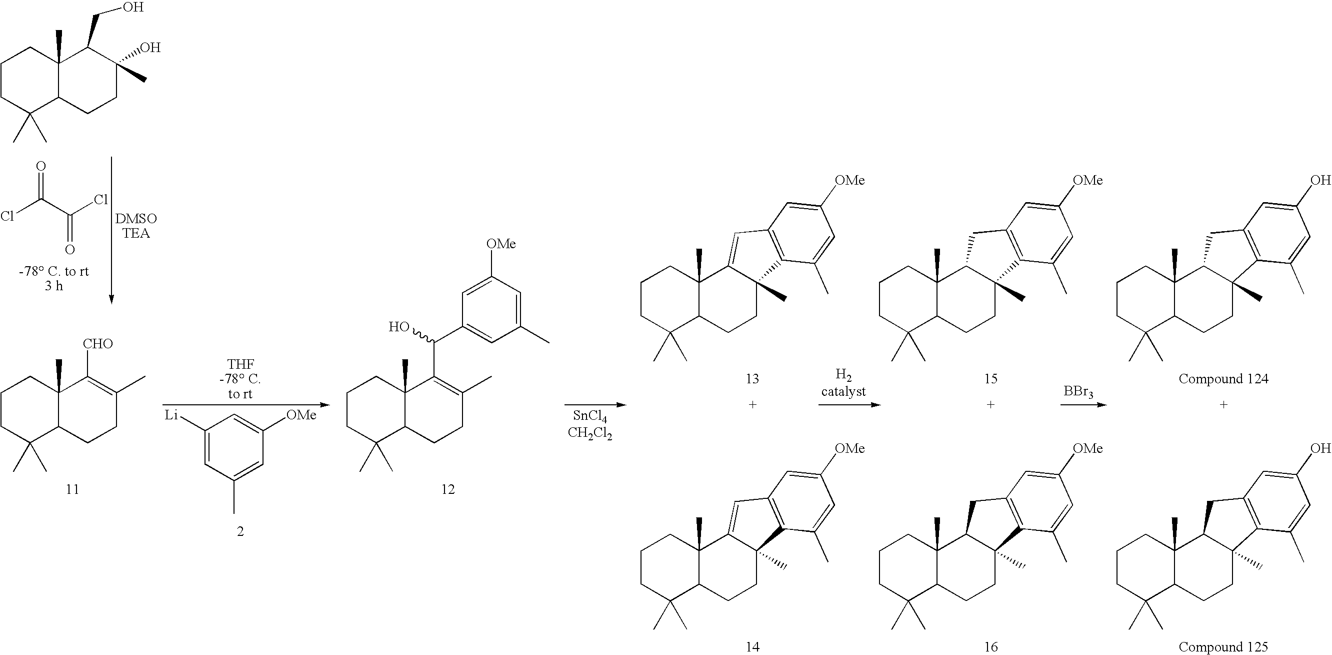

Experimental for preparation of Compound 124 and Compound 125

### Preparation of 12

Bromide 2 (1.41 g, 7.09 mmol) was dissolved in 30 ml dry THF under an argon atmosphere and cooled to −78° C. tBuLi (8.3 ml, 1.7M in pentane, 14.2 mmol) was added over a period of 10 min and the solution was warmed to rt. After 15 min, the solution was recooled to −78° C. and stirred for an additional 30 min. A solution of enal 11 (521 mg, 2.36 mmol) in 8 mL dry THF was then added to the cold solution and the reaction was stirred at −78° C. for 30 min. 1M HCl was then added and the reaction was warmed to rt. The crude product was extracted into EtOAc and washed with satd. NaHCO3. The organic phase was dried over MgSO4, filtered and concentrated. The crude compound was purified by flash chromatography to yield alcohol 12 (451 mg, 1.32 mmol, 56% yield).

¹H NMR (CDCl₃) δ 0.88 (s, 3H), 0.91 (s, 3H), 1.12 (s, 3H), 1.17 (m, 1H), 1.20 (m, 1H), 1.27 (s, 3H), 1.34 (td, 12.9, 3.5 Hz, 1H), 1.41-1.75 (m, 6H), 2.01 (m, 2H), 2.31 (s, 3H), 3.77 (s, 3H), 5.33 (s, 1H), 6.55 (s, 1H), 6.75 (s, 1H), 6.83 (s, 1H)

¹³C NMR (CDCl₃) δ 18.9, 19.1, 20.4, 21.5, 21.7, 21.8, 33.3, 33.4, 34.8, 37.1, 38.9, 41.5, 52.5, 55.1, 69.6, 108.4, 111.7, 118.5, 133.3, 138.9, 143.4, 147.6, 159.6

HRESIMS calcd for C₂₃H₃₄O₂Na 365.2457, found 365.2458

### Preparation of 13 and 14

Alcohol 12 (450 mg, 1.32 mmol) was dissolved in 10 mL CH₂Cl₂ under an argon atmosphere and cooled to −78° C. SnCl₄ (1 mL) was added and the resulting yellow solution was stirred for 15 min. 1M HCl was added to the cold solution and the mixture was allowed to warm to rt. The layers were separated and the organic phase was washed with 2×H₂O, dried over MgSO₄, filtered and concentrated. The crude product was purified by flash chromatography to yield 13 and 14 (284 mg, 0.88 mmol, 67% yield) as a 1:1 mixture.

### Preparation of 15 and 16

A 1:1 mixture of epimers 15 and 16 (84 mg) was dissolved in 5 mL 1:1 MeOH:DMF. 10% Pd/C (32 mg) was added, and the slurry was saturated with H2. The solution was stirred for 16 h under a balloon of H2, after which the solid catalyst was filtered off and washed with EtOAc. The organic phase was washed with 3×H2O, dried over MgSO4, filtered and concentrated to yield 15 and 16 (80 mg, 95% yield) as a 1:1 mixture.

### Preparation of Compound 124 and Compound 125

A 1:1 mixture of 15 and 16 (80 mg, 0.24 mmol) was dissolved in 0.5 mL CH₂Cl₂. BBr₃ (2 mL, 1M in CH₂Cl₂, 2.0 mmol) was added and the solution was stirred at rt for 15 min. The reaction was quenched with slow addition of MeOH, and the crude reaction mixture was concentrated under vacuum. The crude product was purified by flash chromatography to yield a 1:1 mixture of Compound 124 and Compound 125. Compound 124 was fractionally crystallized from the mixture by cooling from toluene.

¹H NMR (CDCl₃) δ 0.87 (s, 3H), 0.89 (s, 3H), 1.20 (m, 2H), 1.25 (s, 3H), 1.30-1.45 (m, 7H), 1.62 (s, 3H), 1.70 (m, 1H), 1.85 (dd, 12.0, 8.4 Hz, 1H), 2.01 (m, 1H), 2.33 (s, 3H), 2.73 (dd, 15.5, 8.3 Hz, 1H), 2.78 (m, 1H), 4.51 (s, 1H), 6.39 (s, 1H), 6.50 (s, 1H)

¹³C NMR (CDCl₃) δ 17.9, 19.7, 21.5, 24.1, 25.8, 32.5, 33.1, 33.5, 36.1, 36.2, 37.9, 41.9, 46.4, 47.5, 61.9, 108.4, 115.8, 133.9, 142.4, 143.3, 153.2

HRESIMS calcd for C₂₂H₃₃O [M+H]⁺ 313.2531, found 313.2533

Compound 125 was fractionally crystallized from the enriched remainder from CH₃CN.

¹H NMR (CDCl₃) δ 0.47 (s, 3H), 0.80 (s, 3H), 0.89 (s, 3H), 0.90 (m, 1H), 1.00 (dd, 11.3, 4.4 Hz, 1H), 1.17 (m, 1H), 1.18 (s, 3H), 1.29 (m, 1H), 1.40 (m, 2H), 1.52 (m, 1H), 1.62 (m, 1H), 1.70 (m, 2H), 2.33 (s, 3H), 2.52 (dt, 14.4, 5.5 Hz, 1H), 2.62 (d, 16.9 Hz, 1H), 2.97 (dd, 16.9, 8.0 Hz, 1H), 4.52 (s, 1H), 6.35 (s, 1H), 6.47 (s, 1H)

¹³C NMR (CDCl₃) δ 15.2, 18.0, 19.1, 19.5, 21.4, 30.5, 31.7, 32.8, 32.9, 34.3, 36.9, 40.7, 41.7, 47.7, 52.0, 62.1, 108.3, 115.3, 133.2, 140.7, 145.6, 153.4

HRESIMS calcd for C₂₂H₃₃O [M+H]⁺ 313.2531, found 313.2533

### Example 3

### Synthesis of Compound 105

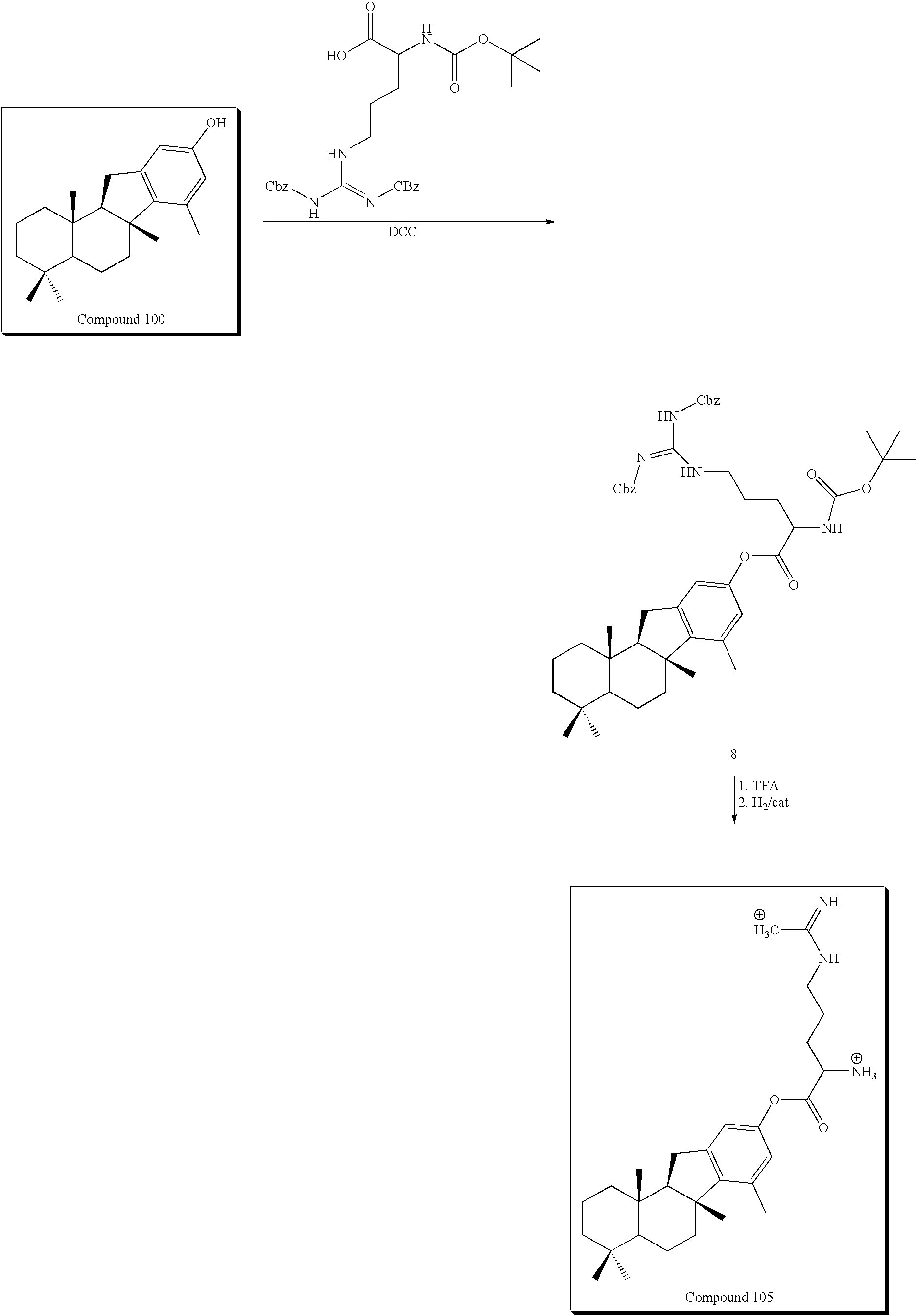

### Experimental for Preparation of Compound 105:

Compound 100 (7), (60.4 mg, 0.193 mmol), Nα-Boc-Nδ,Nω-di-Z-L-Arg-OH (157.3 mg, 0.290 mmol) and DMAP (˜2 mg) were combined in 3 mL CH₂Cl₂. DIPC was added, and the solution was stirred for 2 h at RT. The reaction was concentrated, and purified by flash chromatography to yield 8 as a white foam.

¹H NMR (CDCl₃) δ 0.86 (s, 6H), 0.96 (s, 2H), 1.01 (s, 3H), 1.05 (s, 3H), 1.17 (m, 1H), 1.39 (m, 1H), 1.43 (s, 9H), 1.50 (m, 1H), 1.58 (m, 1H), 1.70 (m, 2H), 1.73 (m, 2H), 1.78 (m, 2H), 1.92 (m, 1H), 2.25 (s, 3H), 2.32 (m, 1H), 2.47 (dd, 14.6, 6.1 Hz, 1H), 2.58 (m, 1H), 4.04 (m, 2H), 4.47 (s, br, 1H), 5.12 (s, 2H), 5.22 (2H), 6.52 (s, 1H), 6.70 (s, 1H), 7.27 (m, 3H), 7.35 (m, 7H), 9.24 (s, br, 1H), 9.45 (s, br, 1H)

¹³C NMR (CDCl₃) δ 14.1, 16.0, 18.2, 18.7, 19.4, 20.0, 20.9, 21.0, 24.9, 28.2 (3C), 28.8, 29.3, 32.9, 33.2, 36.9, 38.5, 40.0, 42.4, 44.1, 47.3, 53.4, 56.9, 60.2, 64.2, 66.9, 68.8, 79.7, 115.5, 120.8, 127.60, 127.62, 128.2, 128.3 (2C), 128.7 (2C), 132.9, 134.6, 136.8, 144.3, 148.1, 149.3, 155.3, 155.7, 160.4, 163.7, 171.5

HRESIMS calcd for C₄₉H₆₅N₄O₈ [M+H]⁺ 837.4802, found 837.4805

### Preparation of Compound 105

Compound 8 was dissolved in 3 mL 70% TFA/CH₂Cl₂ and stirred for 1 h. The solvents were then evaporated, and the resulting residue was redissolved in toluene and concentrated to dryness. The resulting solid was then dissolved in 15 mL MeOH and 100 mg Pd/C (10% wt) was added. The solution was saturated with H₂ and stirred overnight under a hydrogen balloon. The Pd/C was filtered off and the solution was concentrated to dryness. The resulting solid was dissolved in 10 mL H₂O and 50 uL 1M HCl was added. After stirring 5 min, the solution was lyophilized to yield Compound 105 as a white powder.

¹H NMR (CD₃OD) δ 0.86 (s, 3H), 0.87 (s, 3H), 1.01 (m, 2H), 1.05 (s, 3H), 1.08 (s, 3H), 1.19 (td, 13.9, 4.2 Hz, 1H), 1.41 (m, 2H), 1.52 (m, 1H), 1.66 (m, 2H), 1.72 (m, 4H), 1.85 (m, 2H), 2.13 (m, 2H), 2.28 (s, 3H), 2.39 (m, 1H), 2.51 (dd, 14.6, 6.1 Hz, 1H), 2.64 (m, 1H), 4.33 (t, 6.3 Hz, 1H), 6.64 (s, 1H), 6.83 (s, 1H)

¹³C NMR (CD₃OD) δ 16.7, 19.1, 19.3, 20.5, 20.6, 21.5, 25.7, 28.7, 29.8, 33.9, 34.0, 38.2, 40.0, 41.3, 41.6, 43.7, 48.7, 53.7, 58.3, 66.0, 116.5, 121.9, 134.5, 145.8, 149.2, 151.3, 158.8, 169.4

HRESIMS calcd for C₂₈H₄₅N₄O₂ [M+H]⁺ 469.3543, found 469.3540

### Example 4

### Synthesis of Compound 106

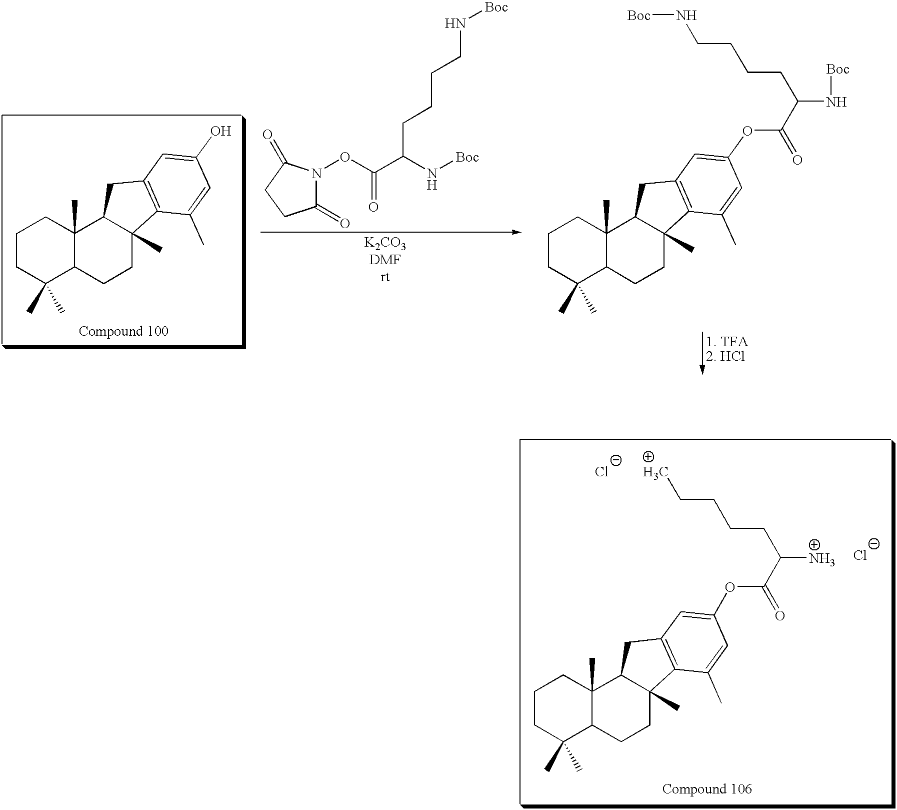

### Experimental for Preparation of Compound 106:

Compound 100 (7) (41.7 mg, 0.133 mmol) was dissolved in 4 mL DMF. K₂CO₃ (37 mg, 0.266 mmol) was added, and the solution was stirred for 10 min. Boc-Lys(Boc)-OSu (115.3 mg, 0.266 mmol) was added, and the solution was stirred for 18 h at RT. The reaction was extracted into EtOAc, and washed with 3×H₂O. The organic phase was dried, filtered and concentrated. The crude product was purified by flash chromatography to yield (23) (80.6 mg, 0.126 mmol, 95% yield) as a white foam.

This foam was dissolved in 2 mL CH₂Cl₂ and TFA (2 mL) was added. The solution was stirred at RT for 2 h, then concentrated to dryness. Toluene (3 mL) was added, and the solution was concentrated to dryness again. The resulting residue was dissolved in 5 mL H₂O and 100 μL 1M HCl was added. The aqueous solution was then filtered through a 0.22 μm syringe filter, and lyophilized to yield Compound 106-2HCl as a white powder.

¹H NMR (CD₃OD) δ 0.82 (s, 3H), 0.83 (s, 3H), 0.96 (m, 2H), 1.01 (s, 3H), 1.04 (s, 3H), 1.16 (td, 13.5, 4.5 Hz, 1H), 1.36 (m, 2H), 1.50 (m, 1H), 1.59 (m, 3H), 1.69 (m, 5H), 2.04 (m, 2H), 2.24 (s, 3H), 2.36 (m, 1H), 2.48 (dd, 14.7, 6.2 Hz, 1H), 2.60 (m, 1H), 2.92 (m, 2H), 3.23 (m, 1H), 4.23 (m, 1H), 6.58 (s, 1H), 6.76 (s, 1H)

¹³C NMR (CD₃OD) δ 16.7, 19.0, 19.4, 20.5, 20.7, 21.5, 23.3, 28.1, 29.8, 31.1, 33.8, 34.0, 38.3, 40.1, 40.3, 41.4, 43.7, 53.9, 58.4, 66.1, 116.5, 121.9, 134.5, 145.9, 149.3, 151.4, 169.4

HRESIMS calcd for C₂₈H₄₅N₂O₂ [M+H]⁺ 441.3481, found 441.3484

### Example 5

### Synthesis of Compound 108

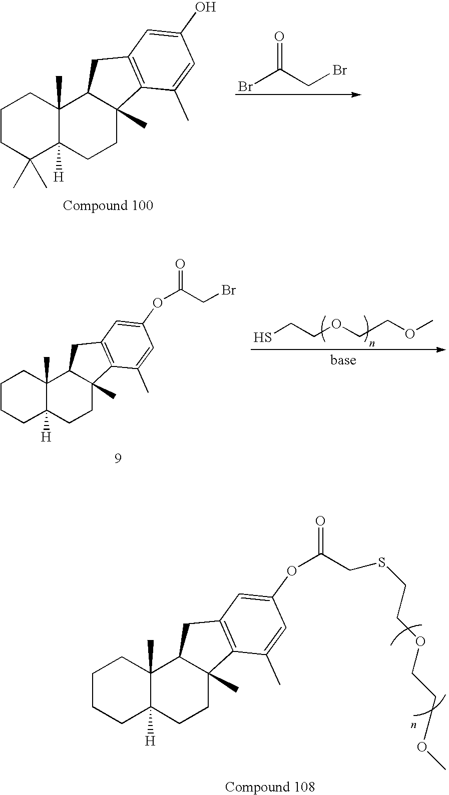

### Experimental for Preparation of Compound 108:

Compound 100 (7: 12.1 mg, 0.039 mmol) was dissolved in 1 mL CH₂Cl₂. DMAP (˜1 mg) was added, followed by Bromoacetyl bromide (5.1 μL, 0.059 mmol), and the reaction was stirred overnight. Concentration of the reaction, followed by flash chromatography yielded bromide (9) (12.9 mg, 0.030 mmol, 77% yield).

¹H NMR (CDCl₃) δ 0.86 (s, 6H), 0.97 (m, 1H), 1.02 (s, 3H), 1.07 (s, 3H), 1.18 (td, 13.5, 4.5 Hz, 1H), 1.25 (s, 1H), 1.41 (m, 2H), 1.52 (m, 1H), 1.60 (m, 1H), 1.71 (m, 2H), 1.77 (m, 2H), 2.29 (s, 3H), 2.35 (m, 1H), 2.52 (dd, 14.6, 6.1 Hz, 1H), 2.62 (m, 1H), 4.00 (s, 2H), 6.59 (s, 1H), 6.78 (s, 1H)

¹³C NMR (CDCl₃) δ 16.1, 18.3, 18.9, 19.5, 20.1, 21.1, 25.7, 28.9, 33.1, 33.3, 37.0, 38.6, 40.1, 42.5, 47.4 57.0, 64.3, 115.3, 120.6, 133.2, 144.6, 148.1, 149.8, 166.2

HRESIMS calcd for C₂₄H₃₃O₂⁷⁹BrNa 455.1562, found 455.1550

HRESIMS calcd for C₂₄H₃₃O₂⁸¹BrNa 457.1541, found 457.1522

Bromide 9 (6.06 g, 11 mmol) was added portionwise over a period of 30 min. to a solution of HS-PEG (35 g, MW 6000) and N,N-diisopropylethylamine (2.7 mL) in acetonitrile (90 mL) under nitrogen at 0° C. After addition, the ice bath was removed and the mixture was allowed to warm to room temperature. After 3-4 hours, 2-propanol (1200 mL) was added over 30 min. After an addition 1.5 h, the resulting solid was collected on a Buchner funnel and washed with 2×150 mL of 2-propanol. The wet cake was then dissolved in acetonitrile (80 mL) containing 0.5% ⁱPr₂NEt at 0-5° C. and precipitated by addition of 2-propanol (1000 mL). The resulting solid was collected and washed with 2-propanol and dried in vacuo to give Compound 108.

### Example 6

### Synthesis of Compound 109

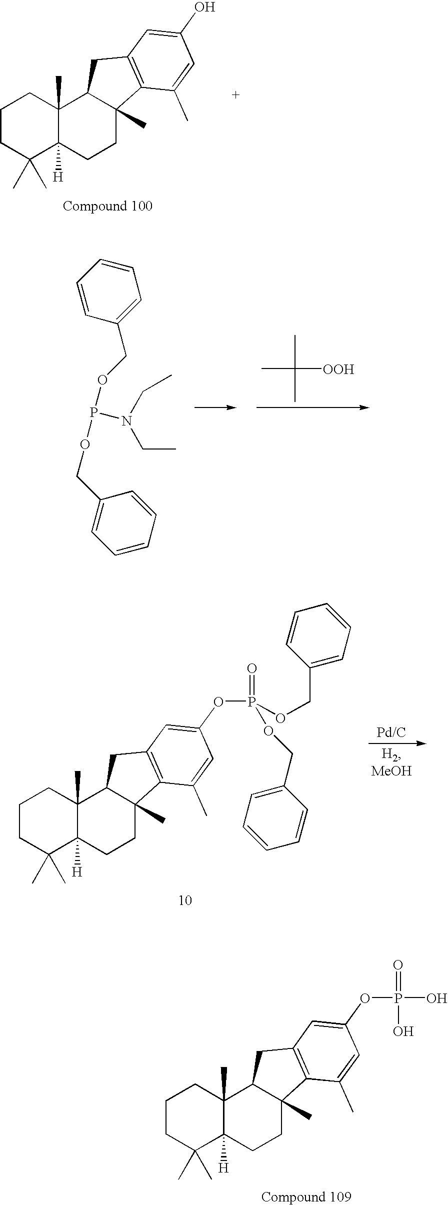

### Experimental for the Synthesis of Compound 109:

General Procedure: Steinberg, G. M. J. Org. Chem. (1950), 15, 637.

Specific tyrosine phosphorylation; Gibson, B. W et al. J. Am. Chem. Soc. (1987), 109, 5343.

Compound 100 (7) (250 mg, 0.80 mmol) was slurried in tetrazole/MeCN solution (18 mL, 8.1 mmol). THF was added until the solution was clear (˜10 mL). To this solution was added dibenzyl N,N-diethylphosphoramidite (1.0 g, 85%, 2.7 mmol), and the reaction was stirred at RT for 1 h. To the reaction was then added 10 mL t-butylhydroperoxide (70% in H₂O) and the solution was stirred vigorously for an additional 30 min. The reaction mixture was extracted into EtOAc, washed with 1×Na₂S₂O₅, 1×1M HCl, then with satd NaHCO₃. The organic layer was dried over MgSO₄, filtered and concentrated. The crude product was purified by flash chromatography to yield 10 (280 mg, 0.49 mmol, 61% yield).

¹H NMR (CDCl₃) δ 0.88 (s, 6H), 0.96 (m, 1H), 0.99 (m, 1H), 1.03 (s, 3H), 1.06 (s, 3H), 1.19 (m, 1H), 1.26 (m, 1H), 1.43 (m, 2H), 1.53 (m, 1H), 1.60 (m, 1H), 1.72 (m, 3H), 2.24 (s, 3H), 2.33 (m, 1H), 2.47 (dd, 14.6, 6.3 Hz, 1H), 2.58 (m, 1H), 5.11 (s, 2H), 5.13 (s, 2H), 6.64 (s, 1H), 6.84 (s, 1H), 7.33 (s, 10H)

¹³C NMR (CDCl₃) δ 16.0, 18.2, 18.7, 19.4, 20.1, 21.0, 28.8, 32.9, 33.3, 36.9, 38.6, 40.0, 42.4, 47.2, 56.9, 64.3, 69.6, 69.7, 114.3, 114.4, 119.7, 119.8, 127.9, 128.4, 133.1, 135.5, 135.6, 144.5, 148.0, 148.1, 148.5

HRESIMS calcd for C₃₆H₄₆O₄P [M+H]⁺ 573.3134, found 573.3117

### Preparation of Compound 109

Compound 10 (280 mg, 0.49 mmol) was dissolved in MeOH (8 mL), and 10% Pd/C was added (30 mg). The solution was saturated with H₂ and stirred for 18 h at RT. The reaction was then filtered through a 0.45 μm membrane and concentrated to dryness to yield Compound 109 (150 mg, 0.38 mmol, 78% yield) as a white powder.

¹H NMR (CD₃OD) δ 0.83 (s, 6H), 0.93 (m, 2H), 1.00 (s, 6H), 1.14 (m, 1H), 1.35 (m, 1H), 1.38 (m, 1H), 1.48 (m, 1H), 1.65 (m, 5H), 2.21 (s, 3H), 2.31 (m, 1H), 2.41 (dd, 14.5, 6.0 Hz, 1H), 2.55 (m, 1H), 6.64 (s, 1H), 6.82 (s, 1H)

¹³C NMR (CD₃OD) δ 16.7, 19.2, 19.4, 20.7, 21.6, 29.8, 33.9, 34.0, 38.2, 40.1, 41.3, 43.7, 48.5, 49.8, 58.4, 66.0, 115.8, 121.3, 134.1, 145.4, 149.1, 150.4

HRESIMS calcd for C₂₂H₃₃O₄NaP 415.2014, found 415.2028

### Example 7

### Synthesis of Compound 103 and Compound 108

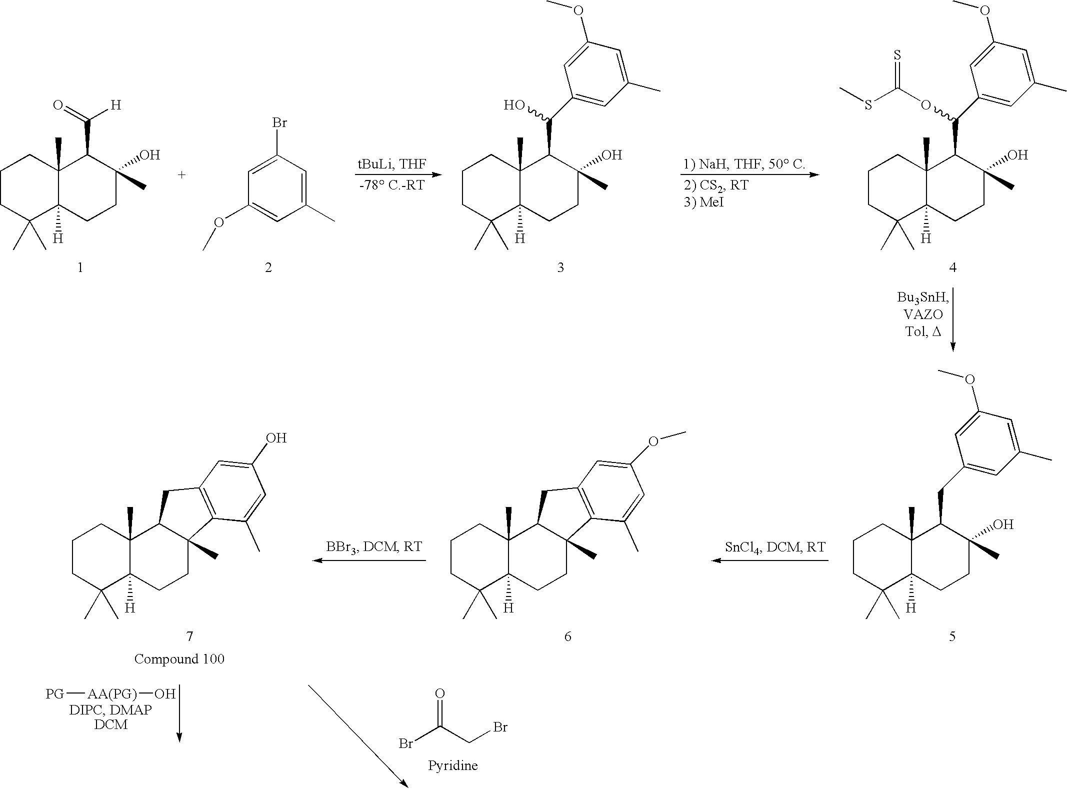

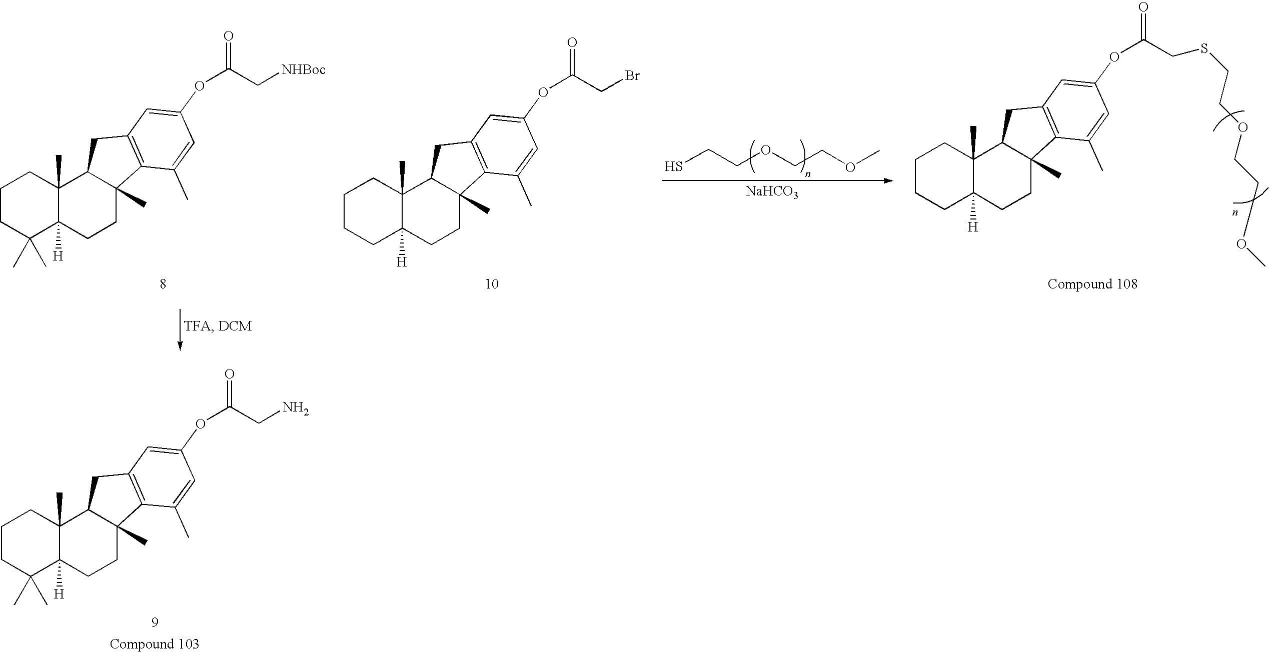

Drimane-8α,11-diol was prepared according to Kuchkova et al; Synthesis, 1997, 1045

Bromomethoxytoluene (2) was prepared according to Chan et al; J. Med. Chem. 44, 1866

### Preparation of Aldehyde (1)

Drimane-8α,11-diol (17.5 g, 72.8 mmol) was dissolved in 1 L CH₂Cl₂. Diisopropylethylamine (50.7 mL, 291.2 mmol) was added and the solution was cooled to −15° C. A solution of Pyr-SO₃ (46.3 g, 291.2 mmol) in DMSO (250 mL) was added dropwise over a period of 20 min, then the reaction was stirred cold for an additional 5 min. To the cold reaction was added 1M HCl (500 mL), and the organic layer was partitioned. The aqueous layer was washed with an additional 200 mL CH₂Cl₂. The pooled organic layers were then washed with saturated NaHCO₃, dried over Na₂SO₄ and concentrated. The crude product was purified by column chromatography (Hex: EtOAc) to yield 10.5 g aldehyde (1) (44.1 mmol, 60.1% yield) as a white semisolid.

### Preparation of Diol (3)

Bromomethoxytoluene (2) (3.64 g, 18.29 mmol) was dissolved in 35 mL dry THF under an argon atmosphere. This solution was cooled to −78° C., and tBuLi (21.5 mL, 36.6 mmol) was added dropwise via syringe. The solution was stirred for 10 min at −78° C., then warmed to RT for 20 min. The solution was re-cooled to −78° C., and a solution of aldehyde (1) (1.45 g, 6.09 mmol) in 6 mL dry THF was added via syringe. The solution was stirred at −78° C. 2 h, after which the reaction was quenched with the addition of 1M HCl. EtOAc (100 mL) was added, and the organic phase was washed with 1M HCl, followed by saturated NaHCO₃. The organic phase was dried over MgSO₄, filtered and concentrated. The crude reaction mixture was purified by column chromatography (Hex:EtOAc) to yield diol (3) (1.94 g, 88.5% yield) as a diastereomeric mixture.

### Preparation of Xanthate (4)

Diol (3) (1.94 g, 5.39 mmol) was dissolved in 20 mL dry THF under an argon atmosphere. To this solution was added NaH (237 mg, 60% in oil, 5.93 mmol). The reaction was then heated to 50° C. until the solution was clear orange. The reaction was cooled to 0° C., and CS₂ (1 mL, 16.6 mmol) was added. The solution was stirred for 20 min at 0° C., then warmed to RT for an additional 20 minutes, after which MeI (1 mL, 16.6 mmol) was added. The reaction was stirred at RT for 1 hour, then concentrated to dryness. The crude mixture was dissolved in EtOAc, and washed with 3×H₂O. The organic solution was dried over MgSO₄, filtered and concentrated to yield a mixture of xanthate (4) and fragmentation product, ketone (12) (approx 4:1). This product mixture was used in the next step without further purification.

### Preparation of Alcohol (5)

Xanthate (4) and ketone (12) were dissolved as a crude mixture in 50 mL toluene, and placed under an argon atmosphere. Bu₃SnH (2.9 mL, 10.78 mmol) was added, and the solution was heated. Once at reflux, a catalytic amount of VAZO (1,1′-Azobis(cyclohexanecarbonitrile)) (approx 50 mg) was added through the top of the condensor. The solution was refluxed for 1 hour, then an additional amount of VAZO was added (approx 50 mg). The solution was refluxed for another 45 min, after which TLC analysis (20% EtOAc:Hex) indicated the reaction to be complete. The reaction was cooled, then concentrated to dryness. Flash chromatography of the crude product yielded alcohol (5) (1.12 g, 3.23 mmol, 60% yield, 2 steps) as a white foam.

### Preparation of Tetracycle (6)

Alcohol (5) (1.12 g, 3.23 mmol) was dissolved in 10 mL CH₂Cl₂ and cooled to 0° C. To this solution was added SnCl₄ (1 mL) neat. The orange solution was then stirred for 1 hour at 0° C., followed by quenching with MeOH. The reaction was extracted into EtOAc, and washed with 2×satd NaHCO₃. The organic phase was dried over MgSO₄, filtered and concentrated to yield tetracycle (6) (1.05 g, 3.20 mmol, 99% yield). This compound was used without further purification.

### Preparation of Compound 100 (7)

Tetracycle (6) (1.05 g, 3.20 mmol) was dissolved in 15 mL DCM. To this solution was added a solution of BBr₃ (1.0M in DCM) (3.20 mL, 3.20 mmol). The solution was stirred at RT for 2 hours, then concentrated to dryness. The brown residue was dissolved in EtOAc, then washed with H₂O until the pH of the aqueous layer was neutral. The crude product was purified by flash chromatography to yield Compound 100 (7) (931 mg, 2.98 mmol, 93% yield) as a white solid.

### Preparation of Glycine Prodrug (9)

Compound 100 (7) (36.1 mg, 0.116 mmol), Boc-Gly-OH (30.5 mg, 0.174 mmol), DMAP (˜2 mg) were combined in 1 mL CH₂Cl₂. 1,3-diisopropylcarbodiimide (27 uL, 0.174 mmol) was added, and the solution was stirred at RT for 2 hours. The reaction was then directly submitted to flash chromatography to yield a white foam (9). This compound was dissolved in 50% TFA/CH₂Cl₂ for 1 hour. The solution was concentrated, redissolved in toluene, and concentrated to dryness. Et₂O (15 mL) was added, and the compound was triturated until the precipitate appeared as a uniform solid. Centrifugation of the mixture, followed by washing of the solid with Et₂O yielded prodrug (9) (44.5 mg, 0.092 mmol, 80% yield) as the TFA salt.

### Preparation of Pegylated Prodrug (Compound 108)

To a solution of (7) (43.0 g, 100 mmol) in acetonitrile at 0° C. were added α-bromoacetic acid (19.46 g, 140 mmol) and DMAP (610 mg, 5 mmol). The reaction mixture was then treated with a solution of DCC (29.87 g, 145 mmol) in acetonitrile (200 mL) dropwise over 30 min, then stirred at 0° C. for 2.5 h. The white solid formed was removed by filtration and washed with acetonitrile (2×100 mL). The combined acetonitrile washes were then added to H₂O (4000 mL) over 15 min. After stirring for another 15 min, the resulting solid was collected and washed with H₂O (2×250 ml) and IPA (2×200 mL) and then dried in vacuo. The collected white solid (6.06 g, 11 mmol) was added portionwise over a period of 30 min. to a solution of HS-PEG (35 g, MW 6000) and N,N-diisopropylethylamine (2.7 mL) in acetonitrile (90 mL) under nitrogen at 0° C. After addition, the ice bath was removed and the mixture was allowed to warm to room temperature. After 3-4 hours, 2-propanol (1200 mL) was added over 30 min. After an addition 1.5 h, the resulting solid was collected on a Buchner funnel and washed with 2×150 mL of 2-propanol. The wet cake was then dissolved in acetonitrile (80 mL) containing 0.5% ⁱPr₂NEt at 0-5° C. and precipitated by addition of 2-propanol (1000 mL). The resulting solid was collected and washed with 2-propanol and dried in vacuo to give Compound 108.

### Example 8

### Synthesis of Compound 102, Compound 103 and Compound 104

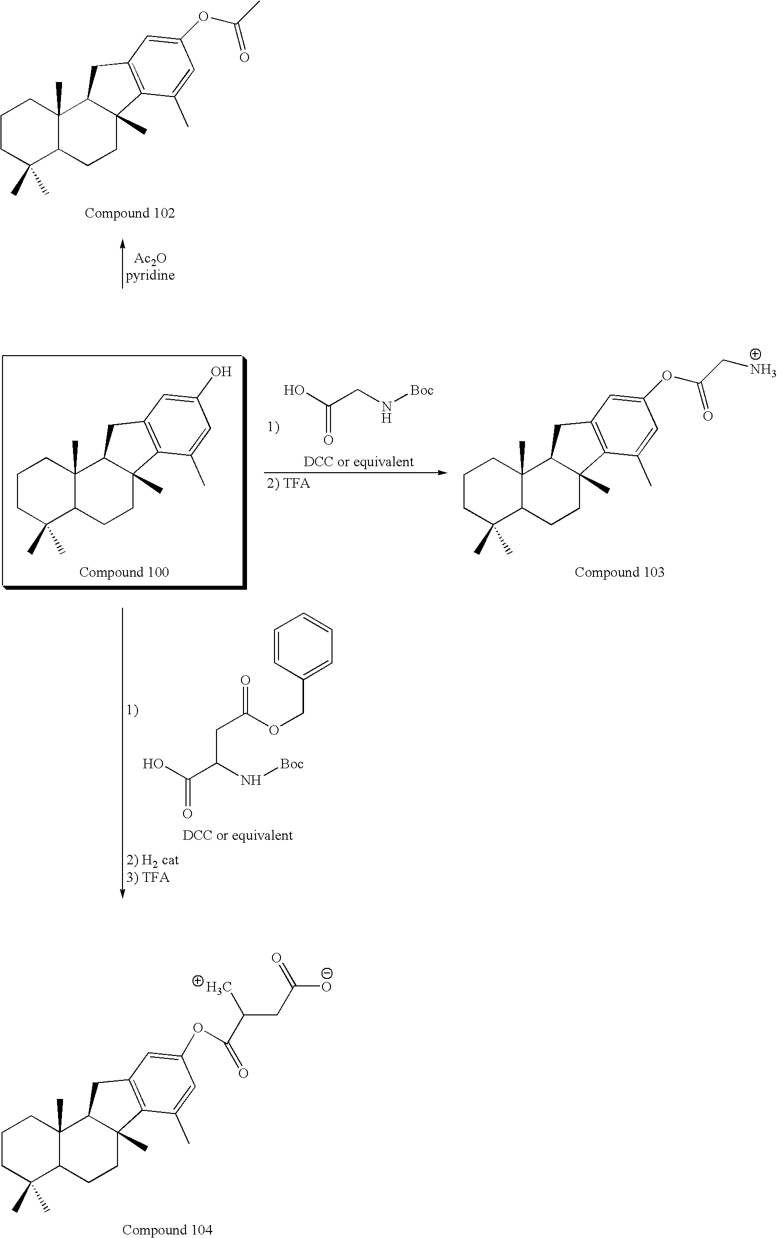

### Example 9

### Compound 103 Inhibits TNF Alpha Production Better than Compound 100

An assay to determine the relative inhibition of TNFα production by Compound 103 compared to Compound 100 was conducted as follows.

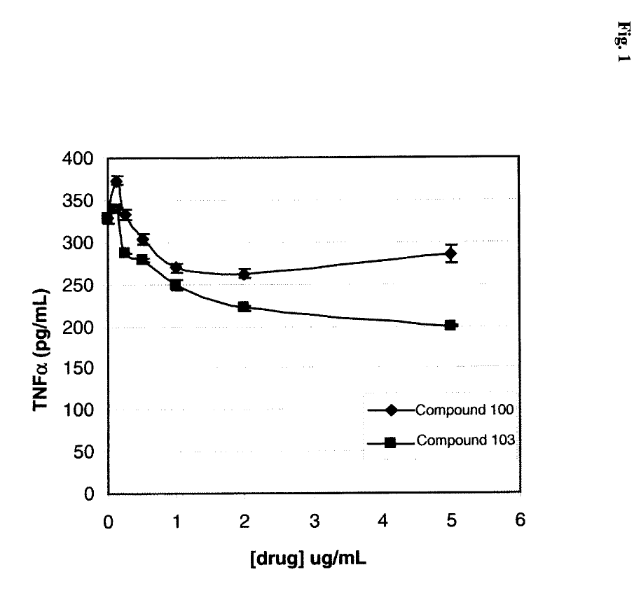

J774.1 macrophage cells were plated at 2×10⁵ cells/well in 24 well plates. The next day the media was changed and Compound 100, Compound 103 or cyclodextrin carrier were added to the wells at the indicated concentrations for 30 min prior to stimulation of the cells with 2 ng/mL lipopolysaccharide (LPS). LPS activation of macrophages leads to production of TNF alpha which can be detected in the culture supernatant and quantified by ELISA. The results are depicted in a graph in FIG. 1.

### Example 10

### Compound 106 Inhibits Macrophage TNFα Production

An assay to determine the inhibition of macrophage TNFα by varying concentrations of Compound 106 was carried out as follows.

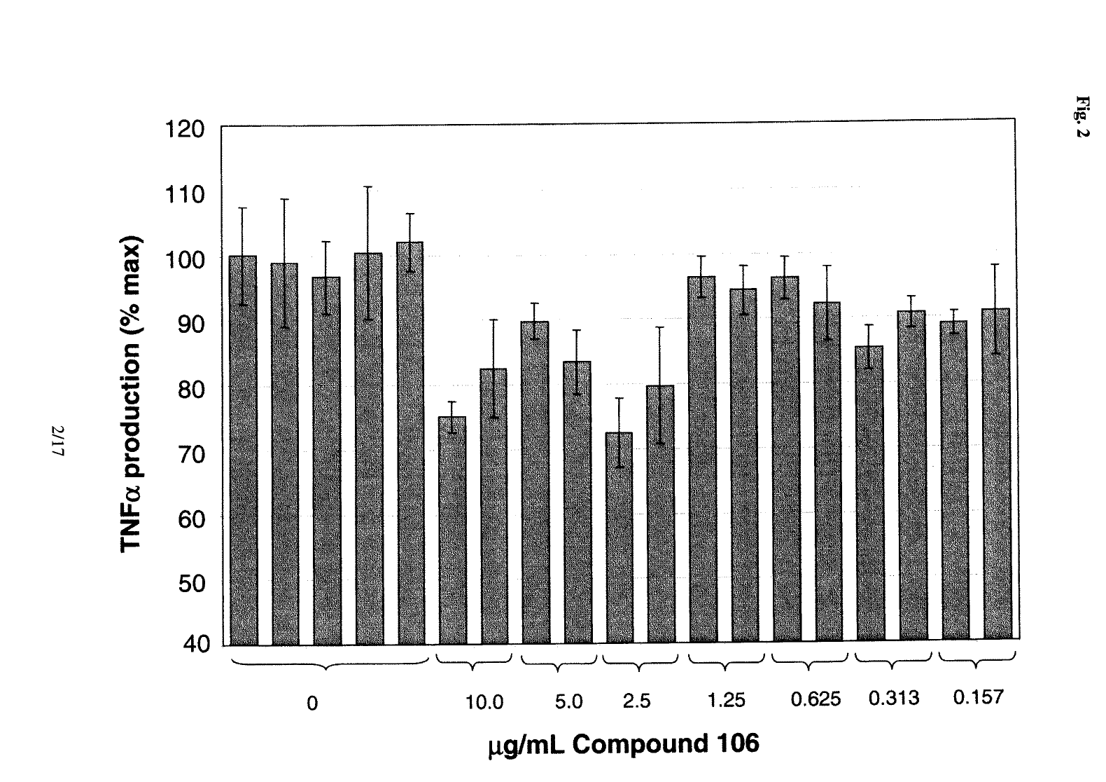

J2M macrophage cells were plated at 2×10⁵ cells/well in 24 well plates. The next day the media was changed and Compound 106 or PBS carrier were added to the wells at the indicated concentrations for 30 min prior to stimulation of the cells with 2 ng/mL lipopolysaccharide (LPS). LPS activation of macrophages leads to production of TNFa which can be detected in the culture supernatant and quantified by ELISA. The results are depicted in a graph in FIG. 2.

### Example 11

### Compound 106 Inhibits Calcium Influx in Mast Cells

An assay to determine the inhibition of calcium influx in mast cells by Compound 106 was carried out as follows.

The activation of mast cells by IgE receptor crosslinking leads to an influx of calcium into the cells, followed by degranulation and secretion of pro-inflammatory mediator. Bone marrow derived mast cells were loaded with the fluorescent calcium indicator dye Fura-2 prior to treatment for 1 hr with Compound 106 or PBS carrier. Cells were then stimulated or not with an anti IgE antibody to cross-link the IgE receptors. Calcium influx was then monitored by fluorimetry. The results are depicted in a graph in FIG. 3.

### Example 12

### Compound 108 Inhibits TNFα Production in Wild Type (WT) but not Knock-Out (KO) Macrophages

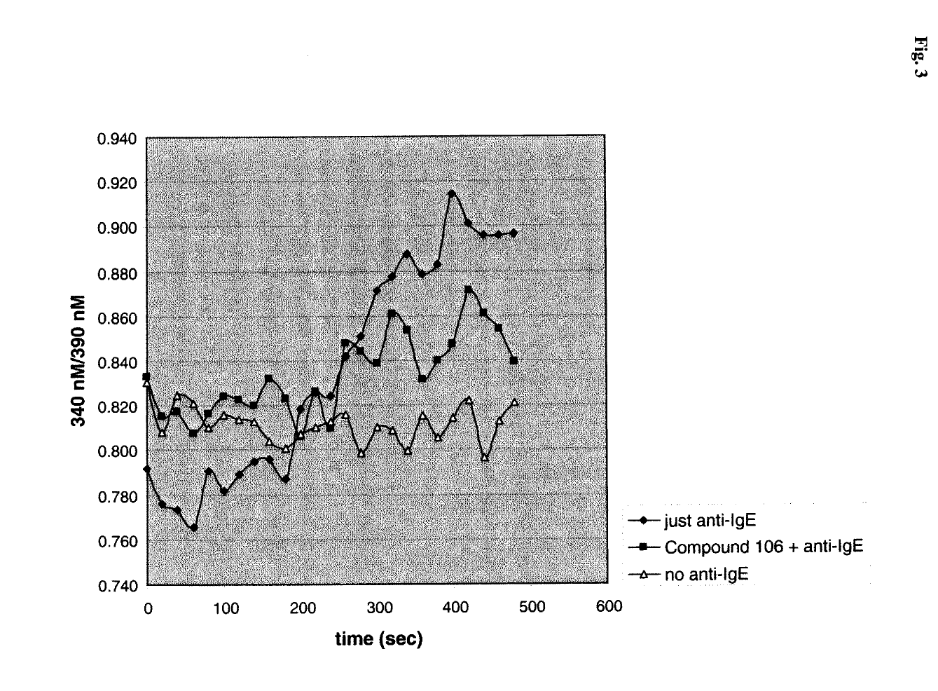

Peritoneal macrophages isolated from wild-type (WT) or SHIP knock-out (KO) mice were in 24 well plates in CSF-1 containing media. The next day the media was changed and Compound 108 or PBS carrier were added to the wells at the indicated concentrations for 60 min prior to stimulation of the cells with 2 ng/mL lipopolysaccharide (LPS). LPS activation of macrophages leads to production of TNF alpha which can be detected in the culture supernatant and quantified by ELISA. The results are depicted in a graph in FIG. 4.

### Example 13

### Assay Screening of SHIP Modulators and Prodrugs Thereof

Various different SHIP modulating compounds and prodrugs thereof were tested in various different assays.

Assay 1) In vitro testing in a SHIP enzyme assay. Test compounds were dissolved in a suitable solvent (e.g. EtOH, DMSO and others) and diluted into aqueous buffer (20 mM Tris HCl, pH 7.5 and 10 mM MgCl₂). SHIP enzyme assays were performed in 96-well microtitre plates with 10 ng of enzyme/well in a total volume of 25 μL of 20 mM Tris HCl, pH 7.5 and 10 mM MgCl₂. SHIP enzyme was incubated with test extracts (provided in solvent) or vehicle for 15 min at 23° C. before the addition of 100 μM inositol-1,3,4,5-tetrakisphosphate (Echelon Biosciences Inc, Salt Lake City, Utah). After 20 min at 37° C. and the amount of inorganic phosphate released assessed by the addition of Malachite Green reagent and absorbance measurement at 650 nm.
Assay 2) Macrophage TNF-α production. J774.1a macrophage cells were treated with 10 μg/mL of test compound dissolved in solvent (e.g. cyclodextran) for 40 minutes prior to the addition of 100 ng/mL LPS. Culture supernatants were collected after 2 hr and 5 hr for TNF-α determination by ELISA.
Assay 3) Macrophage TNF-α NO assay. J774.1a macrophage cells were treated with 10 μg/ml of test compound dissolved in solvent for 40 minutes prior to the addition of LPS. Culture supernatants were collected after 24 hr. for determination of NO concentration using the Griess reagent.
Assay 4) Stimulation of mast cells by FcεRI crosslinking. Mast cells were pre-loaded overnight in BMMC medium lacking IL-3 with 0.1 μg/ml anti-DNP IgE (SPE-7, Sigma, Oakville, Ont). For calcium flux measurements, cells were incubated with 2 μM fura 2-acetoxymethyl ester (Molecular Probes, Eugene, Oreg.) in Tyrode's buffer at 23° C. for 45 min. Cells were then washed and incubated in the presence of the test compound 30 min prior to stimulation with the indicated concentration of DNP-human serum albumin (DNP-HSA). Calcium influx was monitored by spectrofluorometry as described previously. For analysis of intracellular signaling, cells were pre-loaded with anti-DNP IgE as above, pre-treated with the test compound for 30 min at 37° C. and stimulated with 20 ng/ml DNP-HSA for 5 min. Total cell lysates were then prepared and analyzed for phospho-PKB, phospho-p38^{MAPK}, phospho-MAPK, Grb-2 (Cell Signalling, Mississauga, Ont) and SHIP⁶ by immunoblot analysis.
Assay 5) Mouse acute cutaneous anaphylaxis model. 6-8 week old CD1 mice (University of British Columbia Animal Facility, Vancouver, BC) were sensitized to the hapten DNP by cutaneous application of 25 μL of 0.5% dinitrofluorobenzene (DNFB) (Sigma, Oakville, Ont) in acetone to the shaved abdomen of mice for two consecutive days. 24 hrs later, test substances (dissolved in 10 μL of 1:2 DMSO:MeOH) were painted on the right ear while the left ear received vehicle control. 30 min after drug application, DNFB was applied to both ears to induce mast cell degranulation. A 6 mm punch was taken from the ear and immediately frozen on dry ice for subsequent determination of neutrophil myeloperoxidase (MPO) activity.
Assay 6) Mouse endotoxemia model. 6-8 week old C57B16 mice (VCHRI Mammalian Model of Human Disease Core Facility, Vancouver, BC) were orally administered the test compound 30 min prior to an IP injection of 2 mg/kg of LPS (E. Coli serotype 0111:B4, Sigma, Oakville, Ont). Blood was drawn 2 hrs later for determination of plasma TNFα by ELISA.
Assay 7) In vitro Mulitple Myeloma (MM) assay. The ability of SHIP activators to reduce tumor cell survival was assessed in MM cell lines treated with the test compound. The lines OPM1, OPM2, MM.1S and RPMI 8226 were plated at a density of 1×10⁵ cells/mL in 200 μL of medium with various concentrations of the test compound, and viable cell numbers were determined on day 3 and day 5 by trypan blue exclusion. The lines RPMI 8226 and U266 were plated at a density of 1×10⁶ cells/mL in 250 μL of medium with various concentrations of the test compound. At day 4, the medium of each culture was replaced by fresh medium containing the same concentration of test compound. At day 7, the viable cell number of each culture was determined by trypan blue exclusion.

MM cell lines were cultured in 96 well plates seeded with 3×10⁴ cells suspended in 200 μL of medium along with various concentrations of test compound (and associated cyclodextran vehicle control), with LY294002 serving as a positive control in the experiments. After 24-48 hrs of culture, 1 Ci of [3H]-thymidine (GE Healthcare, Baie D'Urfe, Canada) being added for the final 8 hours. Cells were harvested and DNA associated radioactivity was measured via liquid scintillation counting using a Wallac Microbeta counter (Perkin-Elmer; Boston, Mass.).

Assay 8) In vivo Multiple Myeloma (MM) assay. Mice were inoculated with at two sites each with 3×10⁶ luciferase expressing OPM2 cells suspended in 50 μL of growth medium and 50 μL of Matrigel basement membrane matrix (Becton Dickenson; Bedford, Mass.). Tumors were injected subcutaneously in the upper and lower flanks of the mice and allowed to establish for 2 weeks. After 2 weeks, a test compound or control vehicle was administered in a subcutaneous oil depot at a dose of 50 mg/kg every 3 days.

Tumors were measured using bioluminescence imaging on the Xenogen IVIS 200. Mice received intra-peritoneal injections of 200 μL of D-luciferin at 3.75 mg/mL in sterile PBS. Mice were then anesthetized with isofluorane and imaged 15 minutes post-injection of luciferin. Quantification of tumor size was performed using the Living Image™ software.

Various different SHIP modulating compounds and prodrugs thereof were tested in assays as described above and the results are shown quantitatively in the table below, where a ‘+’ indicates a positive result for desired activity, a ‘−’ indicates a negative result for desired activity, and a ‘NT’ indicates no testing.

| Assay Number → | Cmpd | | | | | | | | |
|---|---|---|---|---|---|---|---|---|---|
| Compound Structure ↓ | No. | 1 | 2 | 3 | 4 | 5 | 6 | 7 | 8 |
| 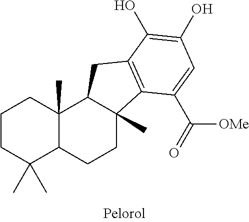 | N/A | + | + | + | NT | NT | NT | NT | NT |
| 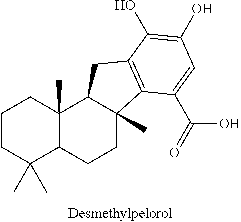 | N/A | + | + | + | NT | NT | NT | NT | NT |
| 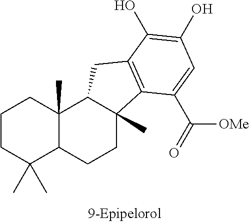 | N/A | + | + | + | NT | NT | NT | NT | NT |
| 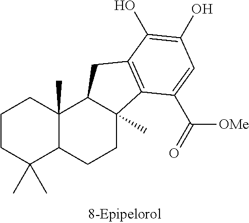 | N/A | + | + | + | NT | NT | NT | NT | NT |
| 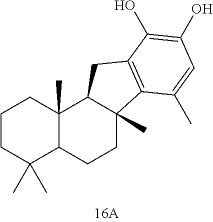 | AQX- 016A | + | + | + | NT | + | + | + | + |
| 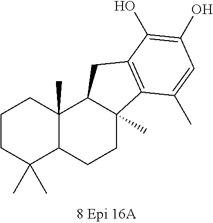 | N/A | + | NT | NT | NT | NT | NT | NT | NT |
| 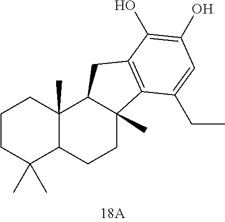 | 18A | + | + | + | NT | NT | NT | NT | NT |
| 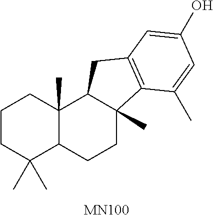 | 100 | + | + | + | + | + | + | + | + |
| 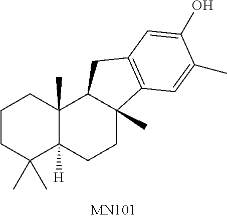 | 101 | NT | + | + | NT | NT | NT | + | NT |
| 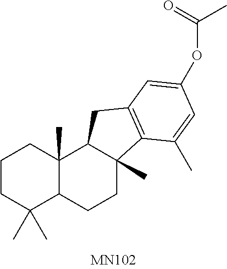 | 102 | NT | + | + | NT | NT | NT | NT | NT |
| 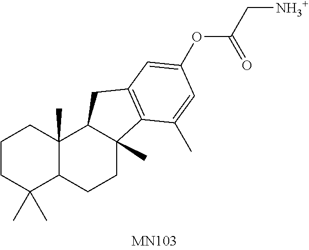 | 103 | NT | + | + | NT | NT | NT | NT | NT |
| 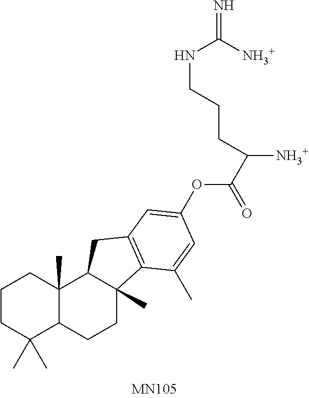 | 105 | NT | + | + | NT | NT | NT | NT | NT |
| 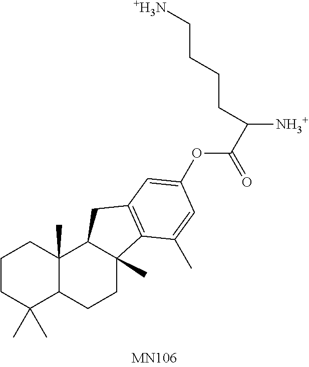 | 106 | NT | + | + | + | NT | + | NT | NT |
| 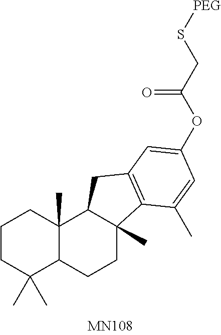 | 108 | NT | + | + | NT | NT | NT | NT | NT |
| 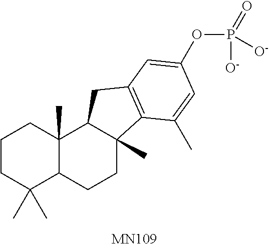 | 109 | NT | − | + | NT | NT | NT | + | NT |
| 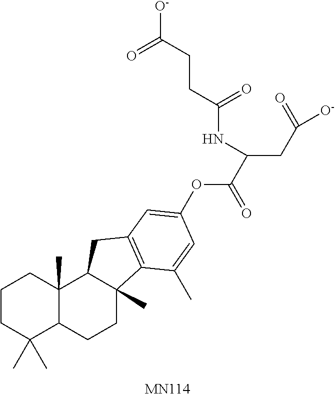 | 114 | NT | + | + | NT | NT | + | NT | NT |
| 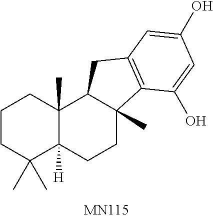 | 115 | NT | NT | NT | NT | NT | NT | + | NT |
| 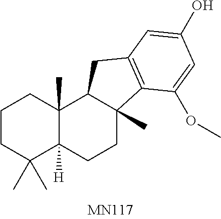 | 117 | NT | + | + | NT | NT | NT | + | NT |
| 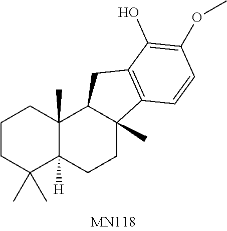 | 118 | NT | − | + | NT | NT | NT | + | NT |
| 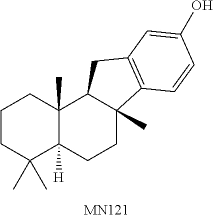 | 121 | NT | + | + | NT | NT | NT | + | NT |
| 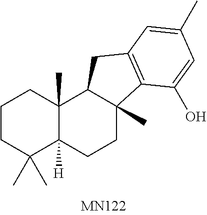 | 122 | NT | − | + | NT | NT | NT | + | NT |
| 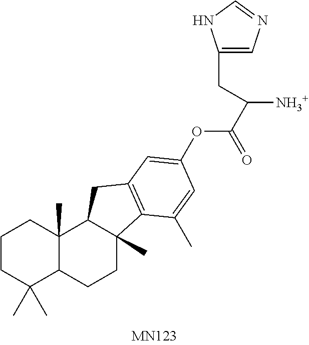 | 123 | NT | + | + | NT | NT | NT | NT | NT |
| 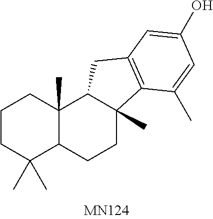 | 124 | NT | + | + | + | NT | NT | + | NT |
| 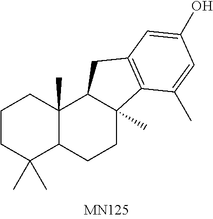 | 125 | NT | + | + | + | NT | NT | + | NT |
| 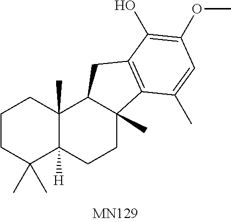 | 129 | NT | − | + | NT | NT | NT | NT | NT |

### Example 14

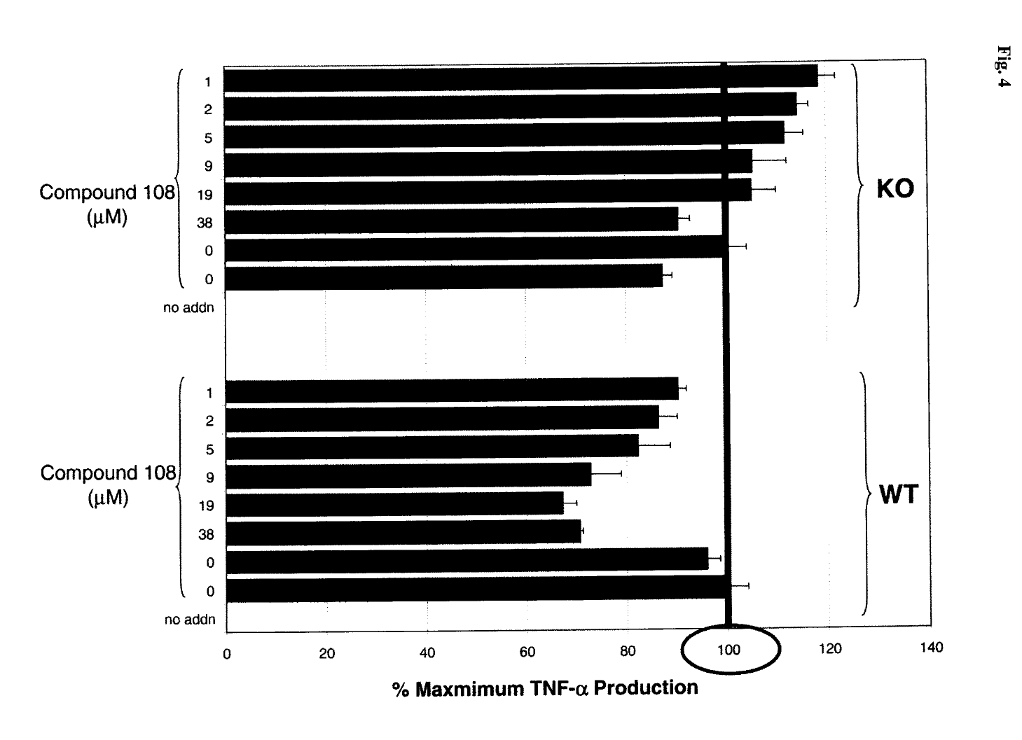

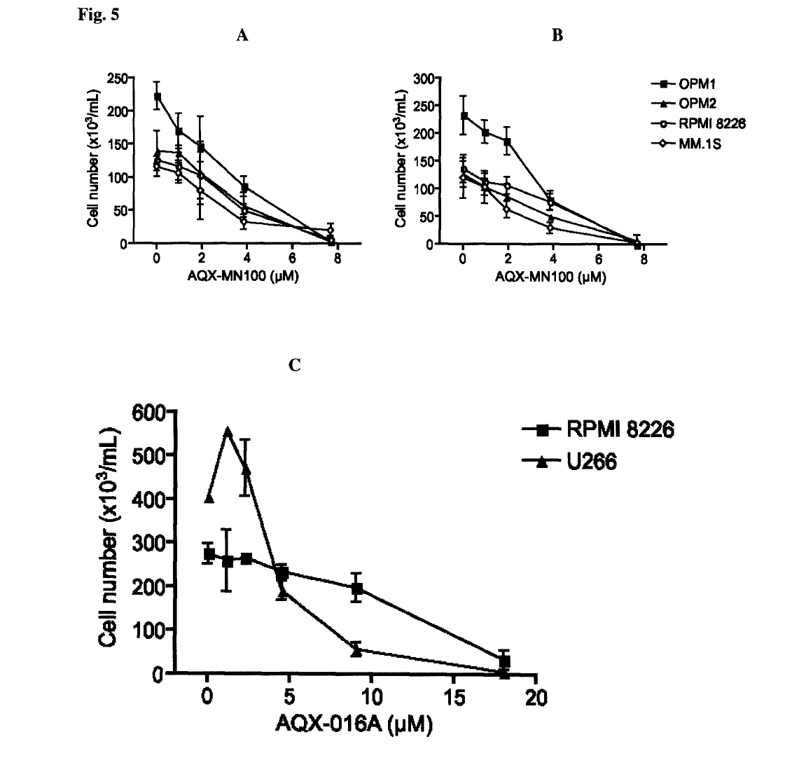

The ability of SHIP activators to reduce tumor cell survival was assessed in multiple myeloma (MM) cell lines treated with Compound 100 or AQX-016A. The lines OPM1, OPM2, MM.1S and RPMI 8226 were plated at a density of 1×10⁵ cells/mL in 200 μL of medium with various concentrations of Compound 100, and viable cell numbers were determined on day 3 and day 5 by trypan blue exclusion. The lines RPMI 8226 and U266 were plated at a density of 1×10⁶ cells/mL in 250 μL of medium with various concentrations of AQX-016A. At day 4, the medium of each culture was replaced by fresh medium containing the same concentration of AQX-016A. At day 7, the viable cell number of each culture was determined by trypan blue exclusion. Experiments were performed in triplicate. Compound 100 inhibits MM proliferation at lower concentrations than AQX-016A and the results are illustrated graphically in FIGS. 5A, 5B and 5C.

### Example 15

Proliferation (DNA synthesis) assays. Proliferation was measured by measuring incorporation of [³H]-thymidine into cells. MM cell lines were cultured in 96 well plates seeded with 3×10⁴ cells suspended in 200 μL of medium along with various concentrations of Compound 100 or AQX-016A (and associated cyclodextran vehicle control), with LY294002 serving as a positive control in the indicated experiments. After 24-48 hrs of culture, 1 μCi of [³H]-thymidine (GE Healthcare, Baie D'Urfe, Canada) being added for the final 8 hours. Plates were frozen, which also aided in cell lysis, to terminate the experiments. Cells were then harvested onto glass fibre filters using an automatic cell harvester (TomTech; Orange, Conn.) and DNA associated radioactivity was measured via liquid scintillation counting using a Wallac Microbeta counter (Perkin-Elmer; Boston, Mass.). Wells were set up in triplicate and data is expressed as mean+/−SEM.

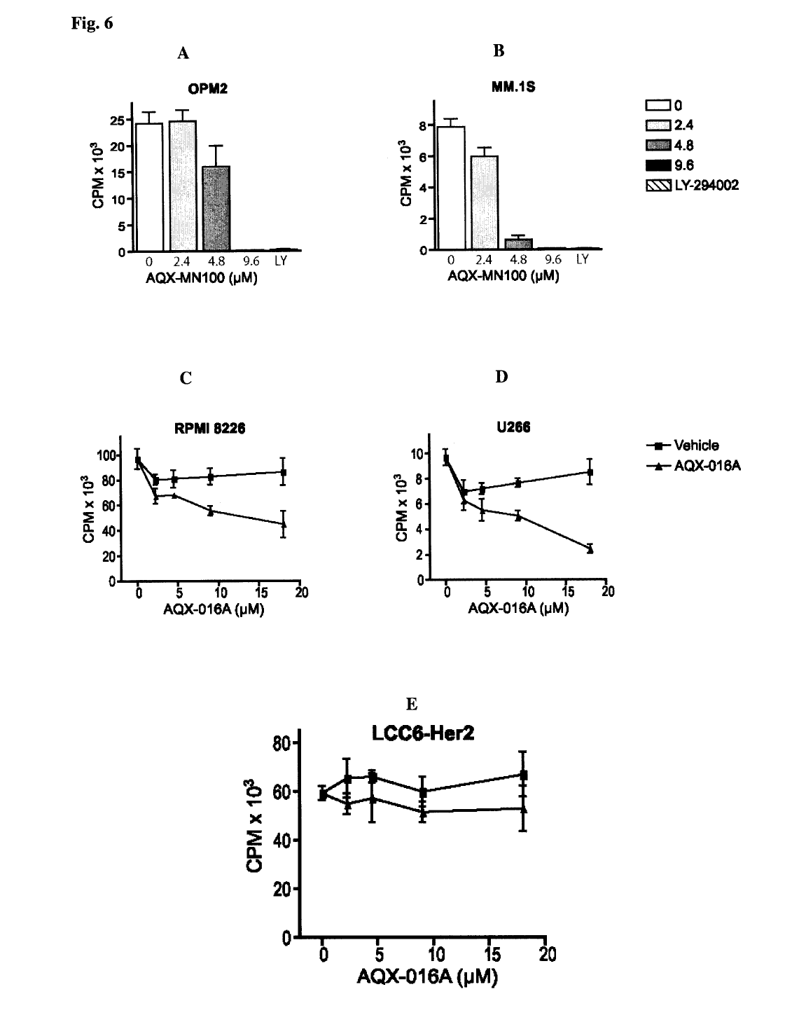

The results are illustrated graphically in FIGS. 6A, 6B, 6C, 6D and 6E.

### Example 16

### Formulation of Compounds

For in vitro testing in the SHIP enzyme assay, AQX-016A and Compound 100 were dissolved in EtOH and diluted into aqueous buffer (20 mM Tris HCl, pH 7.5 and 10 mM MgCl₂). The actual concentration of drug in solution was determined by optical density measurement at 280 nm (λₘₐₓ for both compounds) after high speed centrifugation at 14 000×g for 30 min to remove precipitated drug. For testing on cells, compounds were formulated in the carrier cyclodextrin (Cyclodex Technologies, High Springs, Fla.) at 6 mM (2 mg/mL). For oral administration to animals, compounds were dissolved in 100% cremophore EL (Sigma-Aldrich Canada, Oakville, Ontario) at 150 mM (50 mg/mL) prior to dilution to 6 mM in phosphate buffer saline. However, while these compounds caged in cyclodextrin or formulated in cremophore EL micelles were very soluble in aqueous solution, they could not be used in the SHIP enzyme assays because of interference from both cyclodextrin and cremophore EL.

### Production of Recombinant SHIP Enzyme and SHIP C2 Domain

N-terminal His₆ tagged SHIP enzyme was expressed in mammalian 293T cells by transient transfection with pME18S-His-SHIP plasmid and purified to >95% homogeneity by Ni-chelating bead chromatography (Qiagen, Mississauga, Ontario). Recombinant SHIP C2 domain (amino acid residues 725 to 863) was expressed in E. coli transformed with a pET28C expression vector constructed as described below. Recombinant protein purified from the cell lysates by Nichelating bead chromatography was >95% pure.

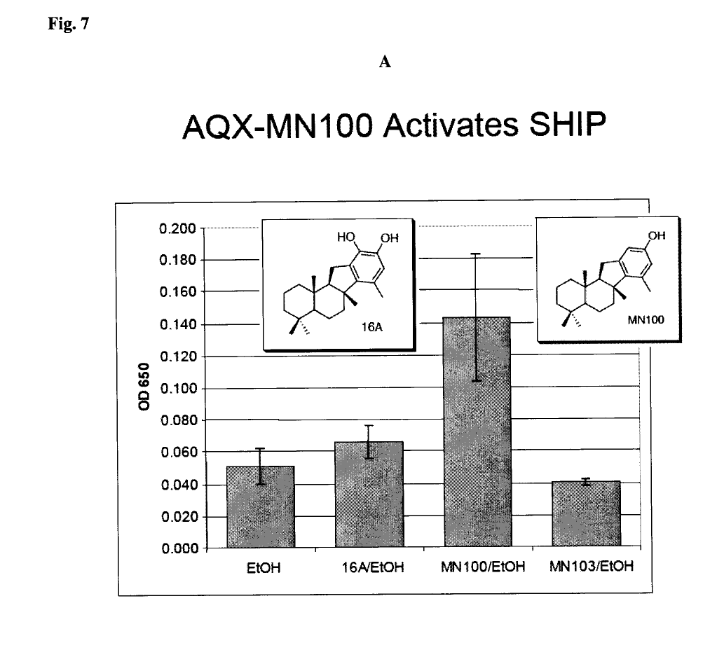

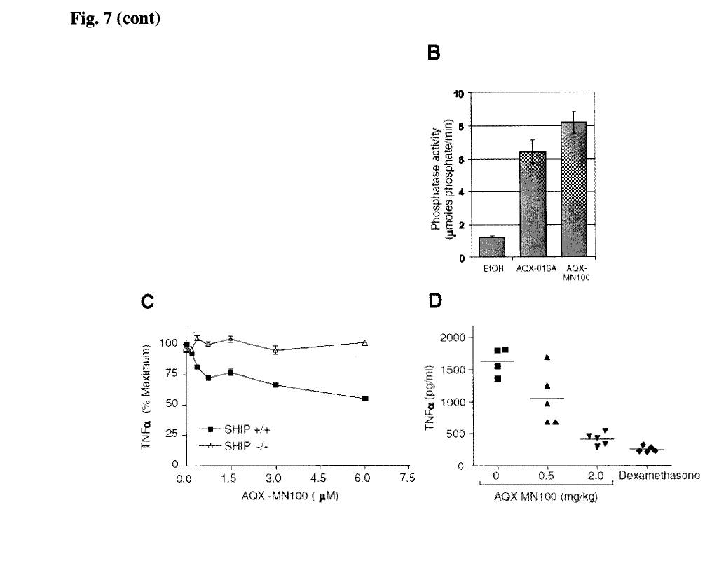

In vitro SHIP enzyme assay. SHIP enzyme assays were performed in 96-well microtitre plates with 10 ng of enzyme/well in a total volume of 25 μL of 20 mM Tris HCl, pH 7.5 and 10 Mm MgCl₂. SHIP enzyme was incubated with test extracts (provided in DMSO) or vehicle for 15 min at 23° C. before the addition of 100 μM inositol-1,3,4,5-tetrakisphosphate (Echelon Biosciences Inc, Salt Lake City, Utah). After 20 min at 37° C. and the amount of inorganic phosphate released assessed by the addition of Malachite Green reagent and absorbance measurement at 650 nm. SHIP2 enzyme was purchased from Echelon Biosciences (Salt Lake City, Utah) and an equivalent amount of inositol phosphatase activity was used in the in vitro enzyme assay. Enzyme data are expressed as the mean of triplicates +/−SEM. Experiments were performed at least 3 times. (FIGS. 7A and 7B).

### Compound 100 is as Biologically Active as AQX-016A at Lower Concentratons

AQX-016A was substantially more active on SHIP^{+/+} than SHIP^{-/-} cells indicates that AQX-016A specifically targets SHIP. However, the presence of a catechol moiety within AQX-016A (FIG. 7A) was potentially problematic since catechols can exhibit activities independent of their specific protein pocket binding interaction For example, catechols can bind metals or be oxidized to an ortho-quinone which can lead to covalent modification of proteins through redox reactions. A non-catechol version of AQX-016A designated Compound 100 (Nodwell M. and Andersen R J, manuscript in preparation). Analogous to AQX-016A, Compound 100 enhanced SHIP enzyme activity in vitro (FIGS. 7A and 7B). Like AQX-016A, Compound 100 also selectively inhibited TNFα production from SHIP^{+/+} but not SHIP^{-/-} macrophages (FIG. 7C). The EC50 for this inhibition was 0.3-0.6 μM. Oral administration of Compound 100 also efficiently inhibited the LPS-induced elevation of plasma TNFα levels in the mouse endotoxemia model (FIG. 7D).

### Example 17

### Production of SHIP+/+ and SHIP−/− Bone Marrow Derived Macrophages and Mast Cells

Bone marrow cells were aspirated from 4 to 8 week old C57B16×129Sv mixed background mice and SHIP^{+/+} and SHIP^{-/-} mast cells prepared as described previously. Bone marrow derived macrophages from SHIP^{+/+} and SHIP^{-/-} mice were obtained and maintained in IMDM supplemented with 10% FCS, 150 μM MTG, 2% C127 cell conditioned medium as a source of macrophage colony stimulating factor (M-CSF) (macrophage medium)

LPS stimulation of macrophages. For the analysis of LPS-stimulated TNFα production, 2×10⁵ cells were plated the night before in 24 well plates in macrophage medium. The next day, the medium was changed and AQX-016A or carrier was added to cells at the indicated concentrations for 30 min prior to the addition of 10 ng/mL LPS. Supernatants were collected for TNFα determination by ELISA (BD Biosciences, Mississauga, ON, Canada). For analysis of intracellular signaling, 2×10⁶ cells were plated the night before in 6 cm tissue culture plates. The next day, the cells were cultured in macrophage medium without M-CSF for 1 hr at 37° C. and then pretreated with AQX-016A or carrier for 30 min prior to the addition of 10 ng/mL LPS for 15 min. Cells were washed with 4° C. PBS and resuspended in lysis buffer (50 mM Hepes, 2 mM EDTA, 1 mM NaVO₄, 100 mM NaF, 50 mM NaPPᵢ and 1% NP40) supplemented with Complete Protease Inhibitor Cocktail (Roche, Montreal, Canada). Lysates were rocked at 4° C. for 30 min and clarified by centrifuging 20 min at 12000×g. Lysates were then made 1× in Laemmli's buffer, boiled 2 min and loaded onto 7.5% SDS polyacrylamide cells. Immunoblot analysis for phospho PKB (Cell Signalling, Mississauga, Ont), SHIP and actin (Santa Cruz, Santa Cruz, Calif.) were carried out as described previously.
Stimulation of mast cells by FcεRI crosslinking. Mast cells were pre-loaded overnight in BMMC medium lacking IL-3 with 0.1 μg/ml anti-DNP IgE (SPE-7, Sigma, Oakville, Ont). For calcium flux measurements, cells were incubated with 2 μM fura 2-acetoxymethyl ester (Molecular Probes, Eugene, Oreg.) in Tyrode's buffer at 23° C. for 45 min. Cells were then washed and incubated in the presence of vehicle control, LY294002 or AQX-016A 30 min prior to stimulation with the indicated concentration of DNP-human serum albumin (DNP-HSA). Calcium influx was monitored by spectrofluorometry. For analysis of intracellular signaling, cells were pre-loaded with anti-DNP IgE as above, pre-treated with AQX-016A or buffer control for 30 min at 37° C. and stimulated with 20 ng/ml DNP-HSA for 5 min. Total cell lysates were then prepared and analyzed for phospho-PKB, phospho-p38^{MAPK}, phospho-MAPK, Grb-2 (Cell Signalling, Mississauga, Ont) and SHIP⁶ by immunoblot analysis.

### AQX-016A Inhibits Macrophage and Mast Cell Activation

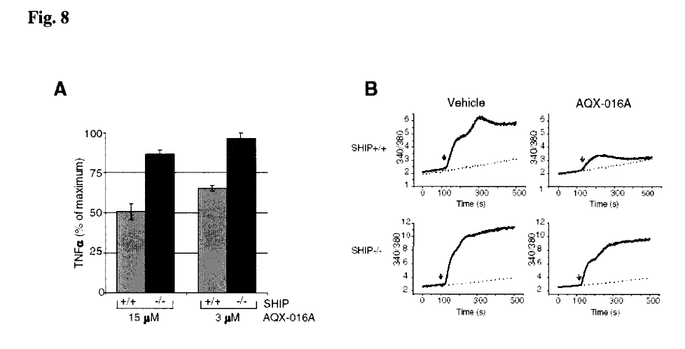

The target specificity and biological efficacy of AQX-016A were assessed by comparing AQX-016A's effects on PI3K-regulated processes in primary SHIP^{+/+} vs SHIP^{-/-} macrophages and mast cells. Both LPS-induced macrophage and IgE-induced mast cell activation involve activation of PI3K-dependent pathways which have previously been shown to be negatively regulated by SHIP. LPS stimulation of macrophages is associated with a PIP3-dependent release of pro-inflammatory mediators such as TNFα. The action of AQX-016A on SHIP^{+/+} vs SHIP^{-/-} bone marrow derived macrophages was examined. Cells were pretreated for 30 min with AQX-016A prior to stimulation with 10 ng/mL of LPS for 2 h. AQX-016A was able to suppress TNFα production in SHIP^{+/+} cells by 30% at 3 μM and 50% at 15 μM (FIG. 8A). In contrast, SHIP^{-/-} cells, TNFα production was indistinguishable from non-AQX-016A treated cells 13 at 3 μM and was suppressed 15% at 15 μM. For comparison, the PI3K inhibitor LY294002 inhibited both SHIP^{+/+} and SHIP^{-/-} macrophages to the same extent (up to ˜40% at 15 μM). Activation of mast cells via IgE+ antigen crosslinking of their IgE receptors results in elevation of intracellular calcium levels. As shown in FIG. 8B, AQX-016A selectively inhibited IgE+ antigen-induced calcium entry to a substantially greater degree in SHIP^{+/+} than in SHIP^{-/-} bone marrow derived mast cells whereas LY294002 inhibited both SHIP^{+/+} and SHIP^{-/-} mast cells to the same extent. These data were consistent with AQX-016A inhibiting PI3K-dependent macrophage and mast cell responses in a SHIP-dependent manner.

The ability of AQX-016A to inhibit activation of PIP₃-dependent downstream signalling proteins in SHIP^{+/+} vs SHIP^{-/-} was assessed. LPS stimulation of macrophages results in PKB phosphorylation. AQX-016A preferentially inhibited, in a dose dependent manner, LPS-stimulated PKB phosphorylation in SHIP^{+/+} but not in SHIP^{-/-} macrophages. Similarly, AQX-016A inhibited the phosphorylation of PKB, p38^{MAPK} and ERK in SHIP^{+/+} but not in SHIP^{-/-} mast cells. Similar protein loading was confirmed by reblotting with either antibodies to PKB or Grb2. We also examined the ability of AQX-016A to inhibit PKB activation in non-ematopoietic, prostate epithelial LNCaP cells, which do not express SHIP. The human prostate cancer cell line LNCaP exhibits constitutive activation of PKB due to the loss of PTEN expression. LY294002 efficiently suppressed PKB phosphorylation whereas AQX-016A had no effect at doses up to 60 μM. Thus, AQX-016A inhibits PIP₃-regulated intracellular signal transduction events in SHIP expressing hematopoietic cells, but not in SHIP-deficient hematopoietic or non-hematopoietic cells.

### Example 18

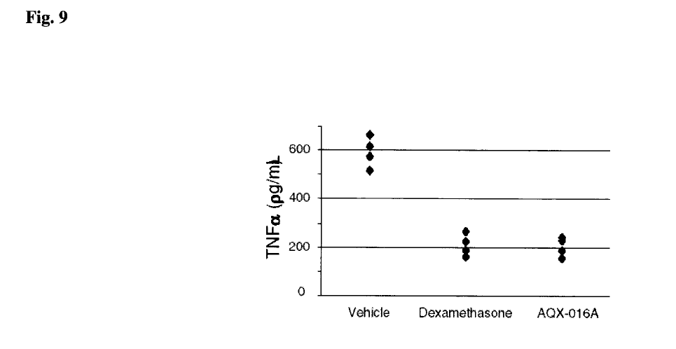

Mouse endotoxemia model. 6-8 week old C57B16 mice (VCHRI Mammalian Model of Human Disease Core Facility, Vancouver, BC) were orally administered the indicated dose of AQX-016A, Compound 100 or dexamethasone or carrier 30 min prior to an IP injection of 2 mg/kg of LPS (E. Coli serotype 0111:B4, Sigma, Oakville, Ont). Blood was drawn 2 hrs later for determination of plasma TNFα by ELISA. Results are representative of 3 independent experiments. (FIGS. 7D and 9)

### AQX-016A Inhibits Inflammation In Vivo

AQX-016A's ability to provide protection by inhibiting inflammatory reactions in vivo was assessed in mouse models. The mouse model of endotoxic shock involves intraperitoneal (IP) injection of bacterial LPS and measurement of serum TNFα levels 2 hrs later. We orally administered AQX-016A or the steroidal drug dexamethasone to mice 30 min prior to the LPS challenge. AQX-016A reduced the level of serum TNFα and did so to the same extent as dexamethasone (FIG. 9).

### Example 19

Mouse acute cutaneous anaphylaxis model. 6-8 week old CD1 mice (University of British Columbia Animal Facility, Vancouver, BC) were sensitized to the hapten DNP by cutaneous application of 25 μL of 0.5% dinitrofluorobenzene (DNFB) (Sigma, Oakville, Ont) in acetone to the shaved abdomen of mice for two consecutive days. 24 hrs later, test substances (dissolved in 10 μL of 1:2 DMSO:MeOH) were painted on the right ear while the left ear received vehicle control. 30 min after drug application, DNFB was applied to both ears to induce mast cell degranulation. A 6 mm punch was taken from the ear and immediately frozen on dry ice for subsequent determination of neutrophil myeloperoxidase (MPO) activity. Compound 100's ability to inhibit cutaneous anaphylaxis was assessed.

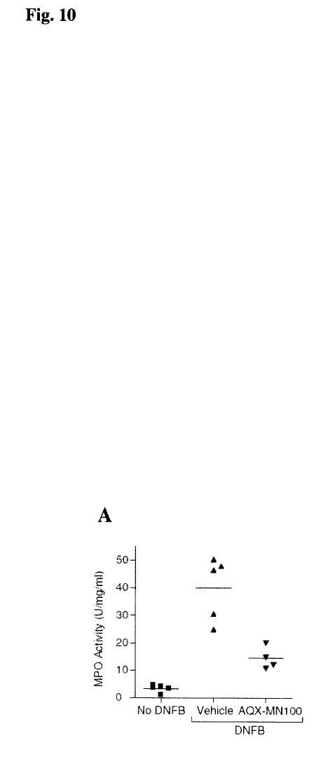

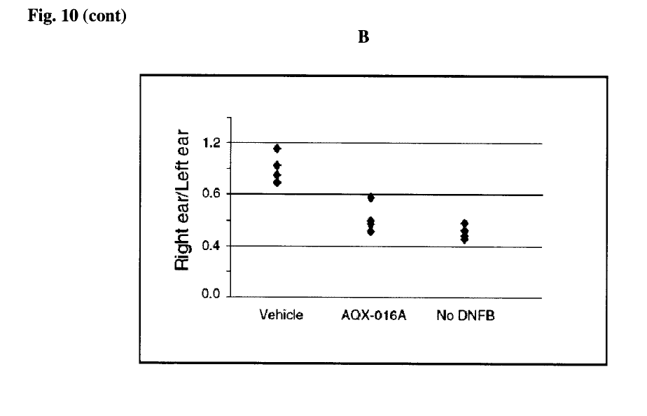

Anaphylactic or allergic responses are mediated by allergen-induced degranulation of pre-sensitized mast cells. The mouse ear edema/cutaneous anaphylaxis model involves pre-sensitization of mice with the haptenizing agent dinitrofluorobenzene (DNFB). One week later the allergic reaction is elicited by painting DNFB onto the ears of the mice. The efficacy of potential anti-inflammatory compounds is tested by topical application of the test substance to one ear and comparing the resulting ear edema or inflammation of the two ears. As shown in FIG. 10A, topically applied Compound 100 dramatically inhibited allergen-induced inflammation compared to the vehicle control-treated ear. AQX-016A was also able to inhibit DNFB-induced inflammation in this model.

AQX-016A inhibited mast cell degranulation in CD1 mice sensitized to hapten DNP by cutaneous application of 25 μL of 0.5% (DNFB) in acetone to the shaved abdomen of mice for two consecutive days was also shown (FIG. 10B). 20 μCi of tritiated thymidine ([³H]-Tdr (GE Healthcare, Piscataway, N.J.) was injected IP one week after the first DNFB application. [³H]-Tdr labels rapidly dividing cells of the mouse, including neutrophils (30). 24 hrs later, test substances (dissolved in 10 μL of 1:2 DMSO:MeOH) were painted on the right ear while the left ear received vehicle control. 30 min after drug application, DNFB was applied to both ears to induce mast cell degranulation. The resulting inflammatory cell infiltration was quantified by taking a 6 mm diameter punch from the ear 1 hr later for dissolution in Solvable (Perkin Elmer-Packard, Woodbridge, Ont) and liquid scintillation counting as described. The ability of test substances to inhibit mast cell degranulation was then determined by calculating the ratio of [³H]-Tdr in the test (right) ear vs the control (left) ear as described (30). One group of mice had DNFB applied only to the left ear leaving the right ear noninflamed, in order to control for basal [³H]-Tdr incorporation into ear parenchymal cells.

### Example 20

### Construction of the SHIP ΔC2 Mutant and Isolated C2 Domain

A His6 tagged SHIP ΔC2 domain deletion mutant (deleting residues 725 to 863) in the mammalian expression vector pME18S was generated by a standard PCR-based methodology. An N-terminal His6 C2 domain construct was also generated by PCR inserted into the pET28C bacterial expression vector using EcoRI and NdeI restriction sites.

### Protein Lipid Overlay Assays

Protein lipid overlay (PLO) assays were performed essentially as described with minor modifications. Lyophilized phosphatidylinositol-3,4-bisphosphate diC16 (PIP₂, Echelon Biosciences, Salt Lake City, Utah) was reconstituted in a 2:1.8 solution of methanol and water. PVDF membranes (Millipore, Missisauga, Ont) were initially wetted in methanol for 1 minute, and washed 3×5 min with water, and gently agitated in TBST buffer (20 mM Tris pH 7.5, 0.15 M NaCl (TBS) with 0.05% Tween 20) at 23° C. overnight. Treated membranes were air-dried and dilutions of reconstituted lipids were spotted in 1 μl aliquots to give the indicated amount of PIP2 per membrane spot. Membranes were dried completely and blocked with blocking buffer (3% BSA in TBS with 0.05% NaN3) for 1 h at 23° C. Purified, recombinant C2 domain was diluted into blocking buffer (5 μM final) and treated with 4 μM Compound 100 or EtOH control for 30 min at 23° C. prior to overnight incubation with the PIP2 spotted membranes. Membranes were washed 10 times over 50 min in TBST buffer at 23° C. and incubated with anti-His₆ mouse IgG (Qiagen, Missisauga, Ont) for 1 h at 23° C. Membranes were washed as above and incubated with Alexa Fluor 660 anti-mouse goat anti-mouse IgG (Invitrogen, Burlington, Ont) for 1 h at 23° C. After washing, bound proteins were detected and quantified on a Li-Cor Odyssey scanner (Lincoln, Nebr.).

### SHIP is an Allosterically Activated Enzyme

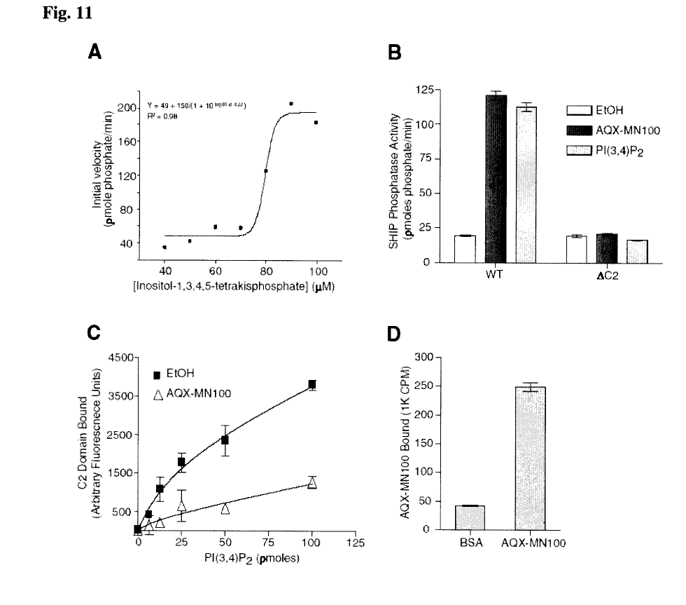

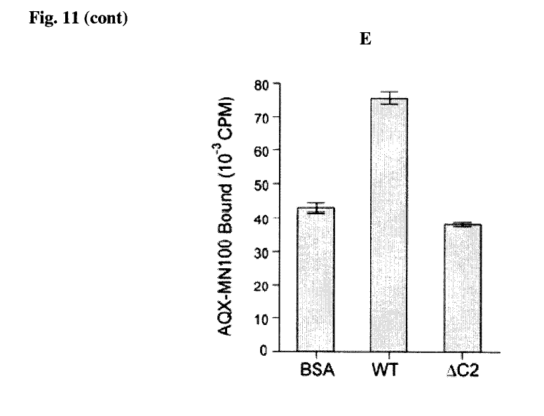

The allosteric regulation of enzymes has remained under-appreciated primarily because allosteric effectors are not easy to find. The molecular mechanism by which Compound 100 activated SHIP was investigated, first by performing classical enzyme kinetic analysis of its phosphatase activity. Activity measurements were performed with substrate concentrations from 10-100 μM. Plots of the initial reaction velocity at each substrate concentration is predicted to exhibit a hyperbolic profile if SHIP obeys conventional Michaelis-Menten kinetics. However, SHIP displayed sigmoidal reaction kinetics suggesting allosteric activation by its end-product (FIG. 11A). Addition of the SHIP product PI-3,4-P₂ to the enzyme reaction activated wild-type SHIP enzyme to a similar extent as Compound 100 (FIG. 11B).

The SHIP protein contains a C2 domain located at the carboxyterminal end of its phosphatase domain. C2 domains were first described in the protein kinase C family where it serves to bind Ca²⁺, but C2 domains have since been identified in other proteins where they have been shown to bind to a variety of ligands including lipids. SHIP lacking its the C2 domain (ΔC2 SHIP) was prepared. As shown in FIG. 11B, although ΔC2 SHIP was as active as the wild-type SHIP, its activity could not be enhanced by the addition of either PI-3,4-P₂ or Compound 100. This suggests that the C2 domain may be required for the allosteric activation of SHIP activity and that it may be the binding site for its allosteric activators such as PI-3,4-P₂ and Compound 100.

### Example 21

### Scintillation Proximity Assays

Compound 100 was radiolabelled with tritium by GE Healthcare (Piscataway, N.J.) to a specific activity of 42 Ci/mmole. Copper chelate (His-Tag) YSi SPA Scintillation Beads (GE ealthcare, Piscataway, N.J.) were diluted in 0.25% BSA/TBS to 1.5 mg/mL and recombinant, His₆-tagged protein added at the indicated concentrations: wild-type (1 pM), ΔC2 SHIP enzyme (1 pM) or C2 domain (10 nM). Protein was allowed to bind 1 h at 23° C., and 250 μg of beads were aliquoted per well of a 96-well plate. 5 μCi of [³H]-Compound 100 was added per well, the plate gently agitated for 30 min and the amount of bead associated radioactivity quantified by counting in a Wallac BetaPlate plate scintillation counter.

Isolated recombinant, His₆-tagged C2 domain was expressed and its PI-3,4-P₂ binding ability was determined using protein lipid overlay assays. Purified C2 domain was incubated with membrane strips spotted with PI-3,4-P₂ and bound protein detected using an anti-His6 antibody. As shown in FIG. 11C the C2 domain bound PI-3,4-P₂ and this binding was inhibited by Compound 100, consistent with both Compound 100 and PI-3,4-P₂ interacting with the C2 domain at a common binding site. Compound 100 was verified to directly bind the C2 domain using scintillation proximity assays (SPAs) in which SPA beads were coated with either the C2 domain or control protein (BSA) prior to incubation with [³H]-Compound 100. As shown in FIG. 11D, the C2 domain did interact with [³H]-Compound 100. In complementary studies, [³H]-Compound 100 bound to wild-type SHIP but not to SHIP lacking its C2 domain (FIG. 11E). Together, these data are consistent with Compound 100 directly binding to SHIP's C2 domain, resulting in allosteric activation of the enzyme.

### Example 22

A novel paradigm for inhibiting PI3K signaling through activation of the phosphatases that negatively regulate this pathway is provided. SHIP is a particularly good target for immune/hematopoietic disorders because of its restricted expression to hematopoietic cells. Because the relative activity of phosphatases present in a cell will influence the efficacy of kinase inhibitors, as discussed by Knight and Shokat, SHIP agonists may also be used to potentiate the activation of PI3K inhibitors and promote tissue targeting of PI3K inhibitors to the ematopoietic/immune cell compartment. Initial toxicology studies suggest both AQX-016A and Compound 100 are well tolerated and do not significantly affect peripheral blood cell counts or bone marrow progenitor numbers (data not shown).

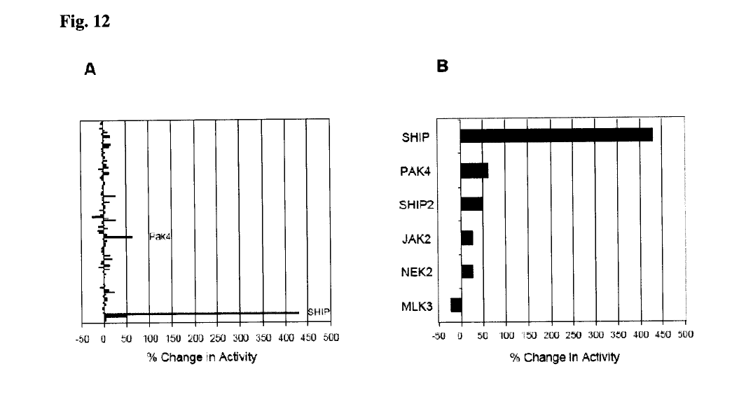

Compound 100 exhibits efficacy at a submicromolar EC50 (FIG. 7C) and this suggests that it possesses a low likelihood of off-target effects based on calculations by Knight and Shokat. Compound 100 had minimal off-target effects on a screen of 100 other kinases and phosphatases (FIGS. 12A and 12B). Compound profiling activity was undertaken using 100 protein kinase and phosphatase targets by SignalChem (Richmond, BC, Canada. www.signalchem.com) against compound Compound 100 (2 μM final concentration). Protein kinase assays were performed in the presence of 50 μM ATP at 30° C. for 15 min. Protein phosphatase activites were determined using pNPP as substrate and were also performed at 37° C. for 15 min. The activity of the enzymes in the presence of Compound 100 was compared to that in the vehicle control and expressed as a % change in activity relative to that observed in the vehicle control. Changes in activity of <25% were not considered significant. Enzymes affected by Compound 100 are plotted in an expanded graph in FIG. 12B.

### Example 23

MM Xenograft murine model. Mice were inoculated with at two sites each with 3×10⁶ luciferase expresseing OPM2 cells suspended in 50 μL of growth medium and 50 μL of Matrigel basement membrane matrix (Becton Dickenson; Bedford, Mass.). Tumors were injected subcutaneously in the upper and lower flanks of the mice and allowed to establish for 2 weeks. After 2 weeks, Compound 100 or control vehicle was administered in a subcutaneous oil depot at a dose of 50 mg/kg every 3 days.

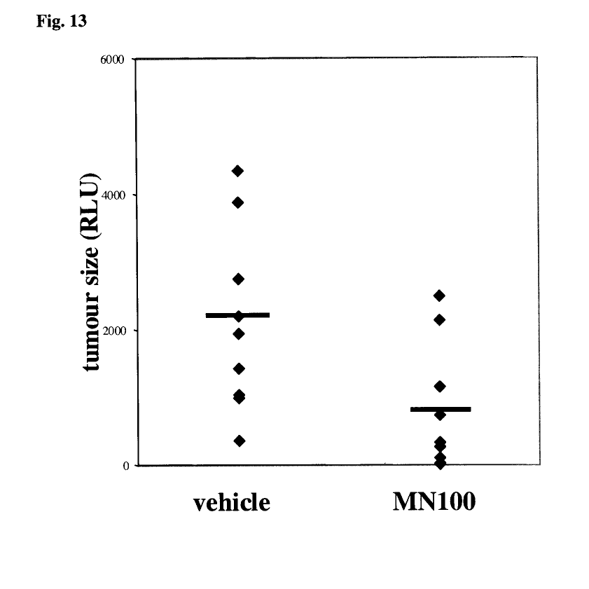

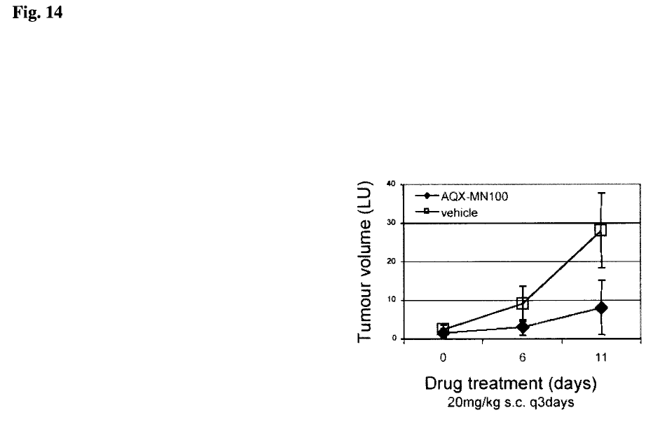

Tumors were measured using bioluminescence imaging on the Xenogen IVIS 200. Mice received intra-peritoneal injections of 200 μL of D-luciferin at 3.75 mg/mL in sterile PBS. Mice were then anesthetized with isofluorane and imaged 15 minutes post-injection of luciferin. Quantification of tumor size was performed using the Living Image™ software. The results are illustrated graphically in FIGS. 13 and 14.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

1-92. (canceled)

93. A compound of Formula II or a salt thereof: 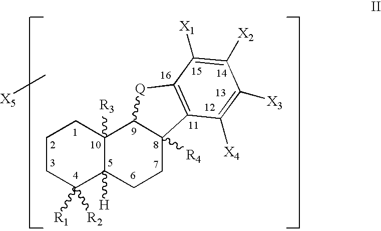
wherein:
R₁ and R₂ are independently selected from the group consisting of: —H, —CH₃, —CH₂CH₃, —CH₂OH, —CH₂OR₁′, —CHO, —CO₂H and —CO₂R₂′;
R₃ and R₄ are independently selected from the group consisting of: —H, —CH₃, —CH₂CH₃, —CH₂OH, —CH₂OR₃′, —CHO, —CO₂H and —CO₂R₄′;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂— and —CY₁Y₂CY₃Y₄CY₅Y₆—; where Y₁, Y₂, Y₃, Y₄, Y₅, and Y₆ are independently selected from the group consisting of: —H, —F, —Br, —Cl, —I, —OH, —OR₅′, —SH, any one group of Y₁/Y₂, Y₃/Y₄ and Y₅/Y₆ are ═O, and Y₁/Y₃ is an epoxide; and at least one of Y₁, Y₂, Y₃, Y₄, Y₅ and Y₆ when present, is not —H;
X₁, X₂, X₃, and X₄ are independently selected from the group consisting of: —H, —X₅, —R₆′, —OH, —O—(C₁-C₁₀ alkyl), —CO₂H, —CO₂R₇′, —F, —Br, —Cl, —I, —CN, —SO₃H, —OSO₃H, —NO₂, —NH₂, —NHR₈′ and —N(R₉′)₂; where R₆′, R₈′ and R₉′ are independently X₅, or a linear, branched or cyclic, saturated or unsaturated, one to ten carbon alkyl group that is unsubstituted or is substituted with one or more of: —X₅, —OH, ═O, —SH, —F, —Br, —Cl, —I, —NH₂, —NHR₁₀′, —N(R₁₁′)₂, —NO₂, —CO₂H, —CO₂R₁₂′ and epoxide;
R₁′, R₂′, R₃′, R₄′, R₅′, R₇′, R₁₀′, R₁₁′ and R₁₂′, are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group; and
X₅ is a prodrug moiety and at least one of R₁, R₂, R₃, R₄, X₁, X₂, X₃ and X₄ are X₅, comprise X₅ as a substituent, or X₅ is a substituent on any carbon atom in Q or in positions 1, 2, 3, 4, 5, 6, 7, 8, 9 and/or 10 of Formula II.

94. A compound of Formula III or a salt thereof: 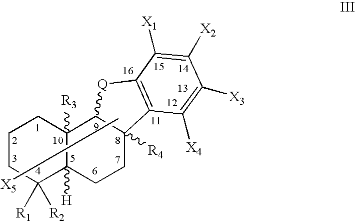
wherein:
R₁ and R₂ are independently selected from the group consisting of: —H, —CH₃, —CH₂CH₃, —CH₂OH, —CH₂OR₁′, —CHO, —CO₂H and —CO₂R₂′;
R₃ and R₄ are independently selected from the group consisting of: —H, —CH₃, —CH₂CH₃, —CH₂OH, —CH₂OR₃′, —CHO, —CO₂H and —CO₂R₄′;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂— and —CY₁Y₂CY₃Y₄CY₅Y₆—; where Y₁, Y₂, Y₃, Y₄, Y₅, and Y₆ are independently selected from the group consisting of: —H, —F, —Br, —Cl, —I, —OH, —OR_{S}', —SH, any one group of Y₁/Y₂, Y₃/Y₄ and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: —H, —R₆′, —OH, —O—(C₁-C₁₀ alkyl), —CO₂H, —CO₂R₇′, —F, —Br, —Cl, —I, —CN, —SO₃H, —OSO₃H, —NO₂, —NH₂, —NHR₈′ and —N(R₉′)₂; where R₆′, R₈′ and R₉′ are independently a linear, branched or cyclic, saturated or unsaturated, one to ten carbon alkyl group that is unsubstituted or is substituted with one or more of: —OH, ═O, —SH, —F, —Br, —Cl, —I, —NH₂, —NHR₁₀′, —N(R₁₁′)₂, —NO₂, —CO₂H, —CO₂R₁₂′ and epoxide;
R₁′, R₂′, R₃′, R₄′, R₅′, V, R₁₀′, R₁₁′ and R₁₂′ are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group; and
X₅ is a prodrug moiety and at least one of R₁, R₂, R₃ and R₄ are X₅, comprise X₅ as a substituent, or X₅ is a substituent on any carbon atom in Q or in positions 1, 2, 3, 4, 5, 6, 7, 8, 9 and/or 10 of Formula III.

95. A compound of Formula IV or a salt thereof: 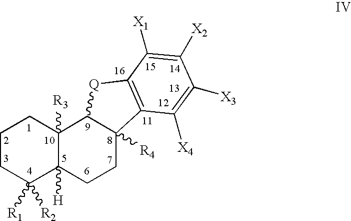
wherein:
R₁ and R₂ are independently selected from the group consisting of: —H, —CH₃, —CH₂CH₃, —CH₂OH, —CH₂OR₁′, —CHO, —CO₂H and —CO₂R₂′;
R₃ and R₄ are independently selected from the group consisting of: —H, —CH₃, —CH₂CH₃, —CH₂OH, —CH₂OR₃′, —CHO, —CO₂H and —CO₂R₄′;
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂— and —CY₁Y₂CY₃Y₄CY₅Y₆—; where Y₁, Y₂, Y₃, Y₄, Y₅, and Y₆ are independently selected from the group consisting of: —H, —F, —Br, —Cl, —I, —OH, —OR₅′, —SH, any one group of Y₁/Y₂, Y₃/Y₄ and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: —H, —X₅, —R₆′, —OH, —O—(C₁-C₁₀ alkyl), —CO₂H, —CO₂R₇′, —F, —Br, —Cl, —I, —CN, —SO₃H, —OSO₃H, —NO₂, —NH₂, —NHR₈′ and —N(R₉′)₂; where R₆′, R₈′ and R₉′ are independently X₅, or a linear, branched or cyclic, saturated or unsaturated, one to ten carbon alkyl group that is unsubstituted or is substituted with one or more of: —X₅, —OH, ═O, —SH, —F, —Br, —Cl, —I, —NH₂, —NHR₁₀′, —N(R₁₁′)₂, —NO₂, —CO₂H, —CO₂R₁₂′ and epoxide;
R₁′, R₂′, R₃′, R₄′, R₅′, R₇′, R₁₀′, R₁₁′ and R₁₂′ are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group; and
X₅ is a prodrug moiety and at least one of X₁, X₂, X₃ and X₄ are X₅ or comprise X₅ as a substituent.

96. The compound of any one of claims 93 to 95 wherein R₁ and R₂ are independently selected from the group consisting of: —CH₃, —CH₂CH₃, —CH₂OH and —CH₂OR_{1′}.

97. A compound of claim 93 having Formula V or a salt thereof: 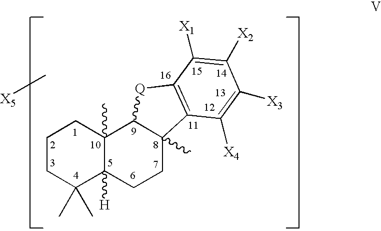
wherein:
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂— and —CY₁Y₂CY₃Y₄CY₅Y₆—; where Y₁, Y₂, Y₃, Y₄, Y₅ and Y₆ are independently selected from the group consisting of: —H, —F, —Br, —Cl, —I, —OH, —OR₅′, —SH, any one group of Y₁/Y₂, Y₃/Y₄ and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: —H, —X₅, —R₆′, —OH, —O—(C₁-C₁₀ alkyl), —CO₂H, —CO₂R₇′, —F, —Br, —Cl, —I, —CN, —SO₃H, —OSO₃H, —NO₂, —NH₂, —NHR₈′ and —N(R₉′)₂; where R₆′, R₈′ and R₉′ are independently X₅, or a linear, branched or cyclic, saturated or unsaturated, one to ten carbon alkyl group that is unsubstituted or is substituted with one or more of: —X₅, —OH, ═O, —SH, —F, —Br, —Cl, —I, —NH₂, —NHR₁₀′, —N(R₁₁′)₂, —NO₂, —CO₂H, —CO₂R₁₂′ and epoxide;
R₅′, R₇′, R₁₀′, R₁₁′ and R₁₂′ are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group; and
X₅ is a prodrug moiety and at least one of X₁, X₂, X₃ and X₄ are X₅, comprise X₅ as a substituent, or X₅ is a substituent on any carbon atom in Q or in positions 1, 2, 3, 4, 5, 6, 7, 8, 9 and/or 10 of Formula V.

98. A compound of claim 94 having Formula VI or a salt thereof: 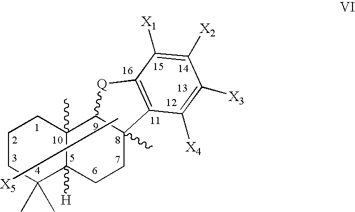
wherein:
Q is selected from the group consisting of: —CH₂—, —CY₁Y₂—, —CH₂CH₂—, —CH═CH—, —CY₁Y₂CY₃Y₄—, —CH₂CH₂CH₂—, —CH═CHCH₂—, —CH═CHCY₁Y₂— and —CY₁Y₂CY₃Y₄CY₅Y₆—; where Y₁, Y₂, Y₃, Y₄, Y₅, and Y₆ are independently selected from the group consisting of: —H, —F, —Br, —Cl, —I, —OH, —OR₅′, —SH, any one group of Y₁/Y₂, Y₃/Y₄ and Y₅/Y₆ are ═O, and Y₁/Y₃ is an epoxide; and at least one of Y₁, Y₂, Y₃, Y₄, Y₅ and Y₆ when present, is not H;
X₁, X₂, X₃, and X₄ are independently selected from the group consisting of: —H, —R₆′, —OH, —O—(C₁-C₁₀ alkyl), —CO₂H, —CO₂R₇′, —F, —Br, —Cl, —I, —CN, —SO₃H, —OSO₃H, —NO₂, —NH₂, —NHR₈′ and —N(R₉′)₂; where R₆′, R₈′ and R₉′ are independently a linear, branched or cyclic, saturated or unsaturated, one to ten carbon alkyl group that is unsubstituted or is substituted with one or more of: —OH, ═O, —SH, —F, —Br, —Cl, —I, —NH₂, —NHR₁₀′, —N(R₁₁′)₂, —NO₂, —CO₂H, —CO₂R₁₂′ and epoxide;
R₅′, R₇′, R₁₀′, R₁₁′ and R₁₂′ are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group; and
X₅ is a prodrug moiety or is a substituent on any carbon atom in Q or in positions 1, 2, 3, 4, 5, 6, 7, 8, 9 and/or 10 of Formula VI.

99. The compound of any one of claims 93 to 95 or a salt thereof wherein Q is selected from the group consisting of —CH₂—, —CY₁Y₂—, —CH₂CH₂—, —CY₁Y₂CY₃Y₄—, —CH₂CH₂CH₂— and —CY₁Y₂CY₃Y₄CY₅Y₆—; where Y₁, Y₂, Y₃, Y₄, Y₅ and Y₆ are independently selected from the group consisting of: —H, —F, —Br, —Cl, —I, —OH, —OR₅′, —SH, any one group of Y₁/Y₂, Y₃/Y₄ and Y₅/Y₆ are ═O, and Y₁/Y₃ is an epoxide; and, at least one of Y₁, Y₂, Y₃, Y₄, Y₅ and Y₆ when present, is not H; and R₅′ is a linear, branched or cyclic, saturated one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated one to ten carbon alkyl group.

100. The compound of any one of claims 93 to 95 or a salt thereof wherein Y₁, Y₂, Y₃, Y₄, Y₅ and Y₆ are H or halogen.

101. The compound of any one of claims 93 to 95 or a salt thereof wherein Q is selected from the group consisting of: —CH₂—, —CH₂—CH₂—, —CH═CH—, —CH₂—CH₂—CH₂— and —CH═CH—CH₂—.

102. A compound of claim 95 having Formula VII or a salt thereof: 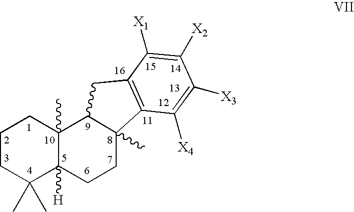
wherein:
X₁, X₂, X₃ and X₄ are independently selected from the group consisting of: —H, —X₅, —R₆′, —OH, —O—(C₁-C₁₀ alkyl), —CO₂H, —CO₂R₇′, —F, —Br, —Cl, —I, —CN, —SO₃H, —OSO₃H, —NO₂, —NH₂, —NHR₈′ and —N(R₉)₂; where R₆′, R₈′ and R₉′ are independently X₅, or a linear, branched or cyclic, saturated or unsaturated, one to ten carbon alkyl group that is unsubstituted or is substituted with one or more of: —X₅, —OH, ═O, —SH, —F, —Br, —Cl, —I, —NH₂, —NHR₁₀′, —N(R₁₁′)₂, —NO₂, —CO₂H, —CO₂R₁₂′ and epoxide;
R₇′, R₁₀′, R₁₁′ and R₁₂′ are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group; and
X₅ is a prodrug moiety and at least one of X₁, X₂, X₃ and X₄ are X₅, or comprise X₅.

103. A compound of claim 102 having Formula VIII or a salt thereof: 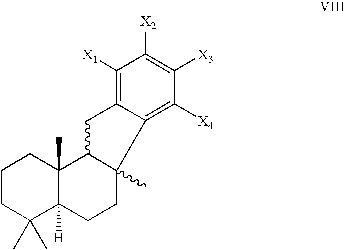
wherein:
X₁, X₂, X₃ and X₄ are independently selected from the group consisting of: —H, —X₅, —R₆′, —OH, —O—(C₁-C₁₀ alkyl), —CO₂H, —CO₂R₇′, —F, —Br, —Cl, —I, —CN, —SO₃H, —OSO₃H, —NO₂, —NH₂, —NHR₈′ and —N(R₉′)₂; where R₆′, R₈′ and R₉′ are independently X₅, or a linear, branched or cyclic, saturated or unsaturated, one to ten carbon alkyl group that is unsubstituted or is substituted with one or more of: —X₅, —OH, ═O, —SH, —F, —Br, —Cl, —I, —NH₂, —NHR₁₀′, —N(R₁₁)₂, —NO₂, —CO₂H, —CO₂R₁₂′ and epoxide;
R₇′, R₁₀′, R₁₁′ and R₁₂′ are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group; and
X₅ is a prodrug moiety and at least one of X₁, X₂, X₃ and X₄ are X₅, or comprise X₅.

104. The compound of any one of claims 93 to 95 or a salt thereof wherein R₆′, R₇′, R₈, and R₉, in at least one of X₁, X₂, X₃ and X₄ is selected from the group consisting of: unsubstituted methyl, unsubstituted ethyl, unsubstituted propyl and unsubstituted butyl.

105. The compound of any one of claims 93 to 95 or a salt thereof wherein at least one of X₁, X₂ and X₃ is selected from the group consisting of: —H, —X₅, —R₆′, —OH, —O—(C₁-C₁₀ alkyl), —F, —Br, —Cl, —I, —CONH₂, —CONHR₁₃′, —CO(R₁₄′)₂, —NHR₈′ and —N(R₉′)₂; where R₁₃′ and R₁₄′ are independently a linear, branched or cyclic, saturated or unsaturated, one to ten carbon alkyl unsubstituted or substituted with one or more of: —OH, ═O, —SH, —F, —Br, —Cl, —I, —NH₂, —NHR₃″, —N(R₄″)₂, —NO₂ and —CO₂H, where R₃″ and R₄″ are independently a linear, branched or cyclic, saturated or unsaturated, one to ten carbon alkyl group.

106. The compound of claim 105 or a salt thereof wherein R₆′, R₈′, R₉′, R₁₃′ and R₁₄′ are selected from the group consisting of: unsubstituted methyl, unsubstituted ethyl, unsubstituted propyl and unsubstituted butyl.

107. The compound of any one of claims 93 to 95 or a salt thereof wherein one or more of X₁, X₂ and X₃ are selected from the group consisting of: —H, —X₅, —OH, —O—(C₁-C₁₀ alkyl), —CONH₂, —CONHR₁₃′ and —CO(R₁₄′)₂, where R₁₃′ and R₁₄′ are independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₃″, —N(R₄″)₂, —NO₂ and —CO₂H, where R₃″ and R₄″ are independently a linear, branched or cyclic, saturated or unsaturated, one to ten carbon alkyl group.

108. The compounds of any one of claims 93 to 95 or salt thereof wherein one or more of X₁, X₂ and X₃ are selected from the group consisting of: —H, —X₅, —OH and —OCH₃.

109. The compound of any one of claims 93 to 95 or salt thereof wherein X₄ is selected from the group consisting of: —H, —X₅, —R₆′, —OH, —O—(C₁-C₁₀ alkyl), —CO₂H and —CO₂R₇′.

110. The compound of any one of claims 93 to 95 or a salt thereof wherein X₅ comprises (a) a solubilizing moiety selected from the group consisting of: a moiety having one or more ionic entities at physiological pH; a moiety having multiple hydrogen bonding functionalities; a monophosphate; a diphosphate; a triphosphate; a monosaccharide; an oligosaccharide; a polysaccharide; an oligopeptide; a polypeptide; an amino acid; an alpha amino acid; a polyether and a combination thereof; and (b) a linking moiety selected from the group consisting of: —O—, —O—C(═O)—Z—, —NH—C(═O)—Z—, —CH₂C(═O)—, —C(═O)O— and —C(═O)NH—, where Z is a linear, branched or cyclic, saturated or unsaturated, one to ten carbon alkyl group that is unsubstituted or is substituted with one or more of: —OH, ═O, —SH, —F, —Br, —Cl, —I, —NH₂, —NHR′, —NR′₂, —NO₂, —CO₂H, —CO₂R′ and epoxide, and individual carbon atoms may be replaced by S, O, N, NR′, or NR′₂ atoms; and each R′ is independently a linear, branched or cyclic, saturated or unsaturated, one to ten carbon alkyl group that is unsubstituted or substituted with one or more of: —OH, ═O, —SH, —F, —Br, —Cl, —I, —NH₂, —NHR₁″, —N(R₂″)₂, —NO₂ and —CO₂H, where R₁″ and R₂″ are independently a linear, branched or cyclic, saturated or unsaturated, one to ten carbon alkyl group.

111. The compound of any one of claims 93 to 95 or a salt thereof wherein X₅ comprises an amide linking moiety, an ester linking moiety, a solubilizing moiety comprising an NH₂ moiety, an amino acid, a phosphate or a polyethylene glycol moiety.

112. A compound having the structure: 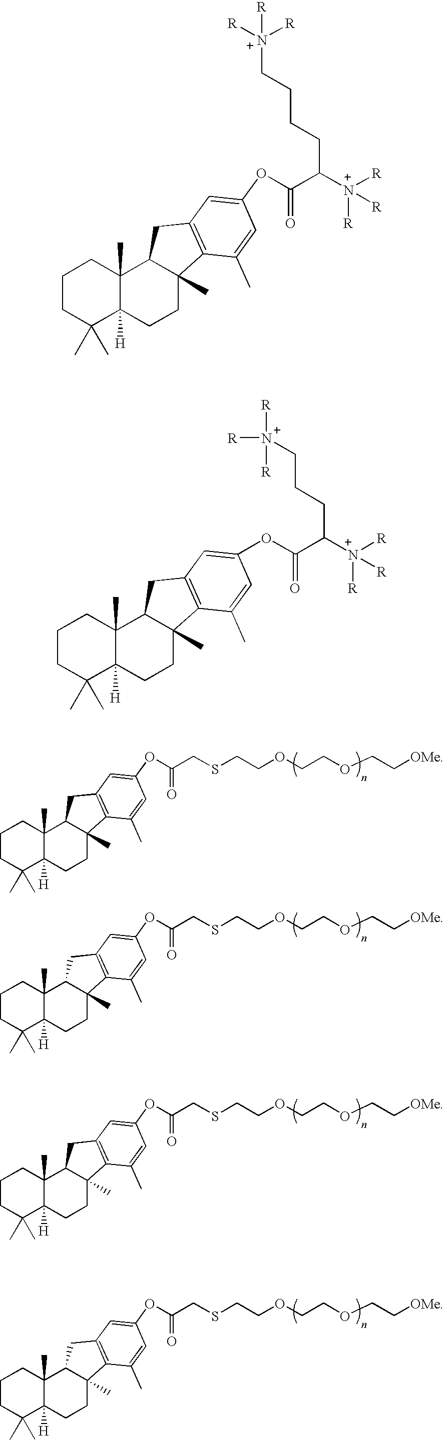 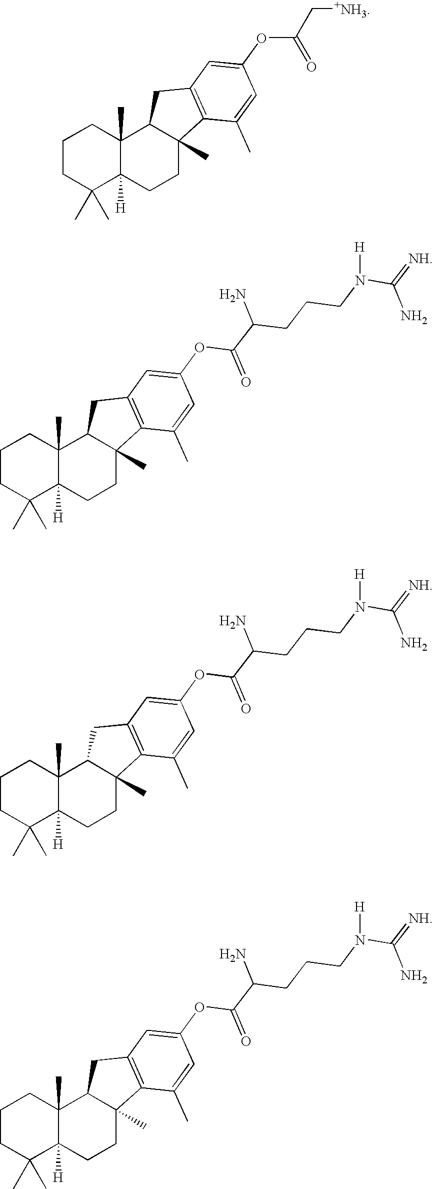 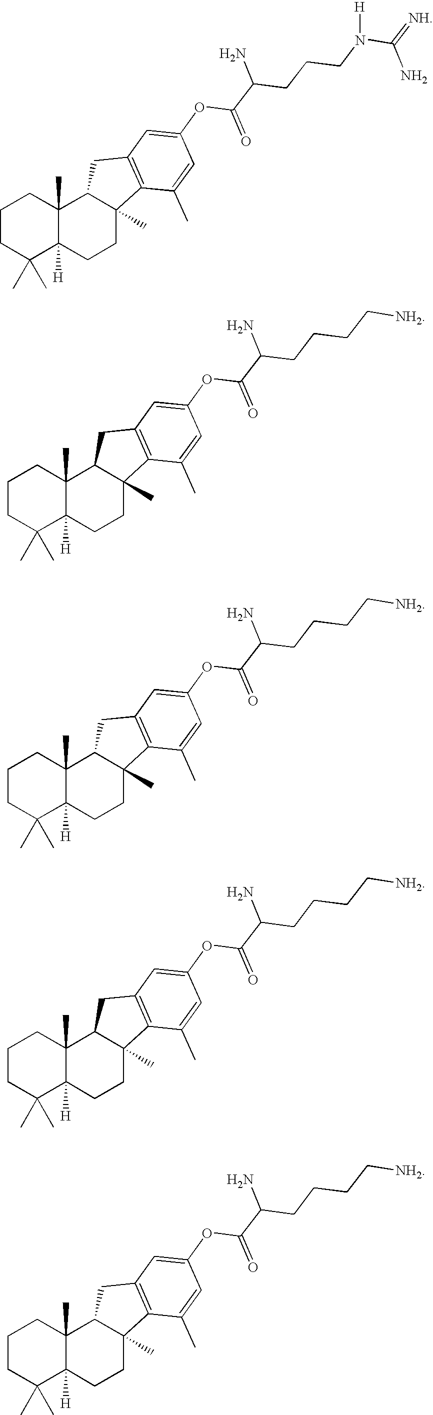 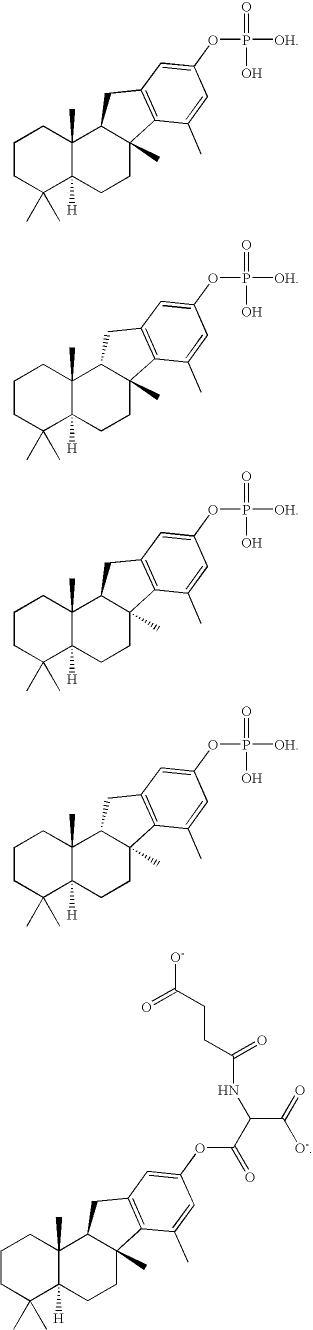 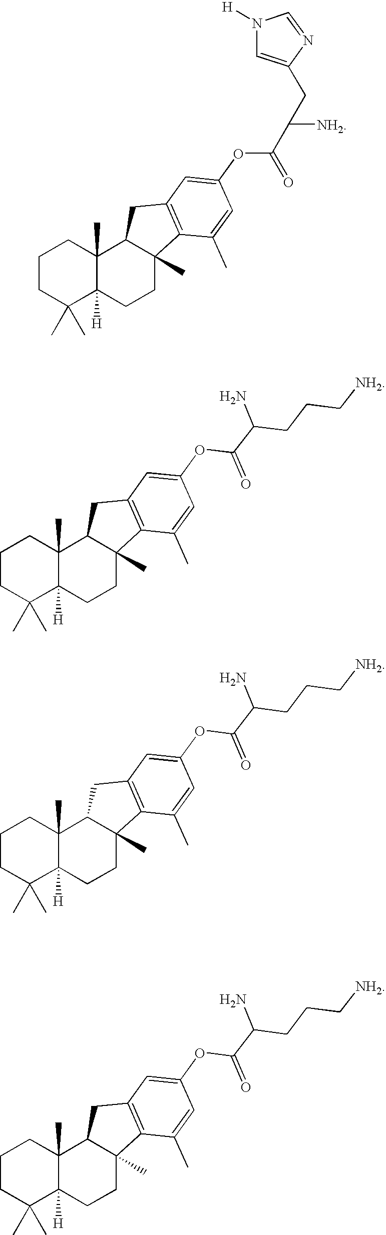 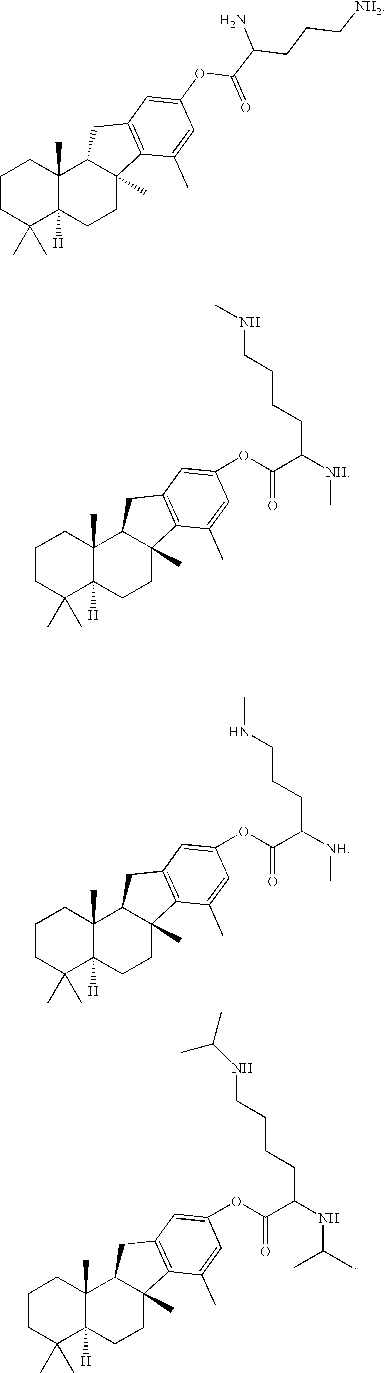 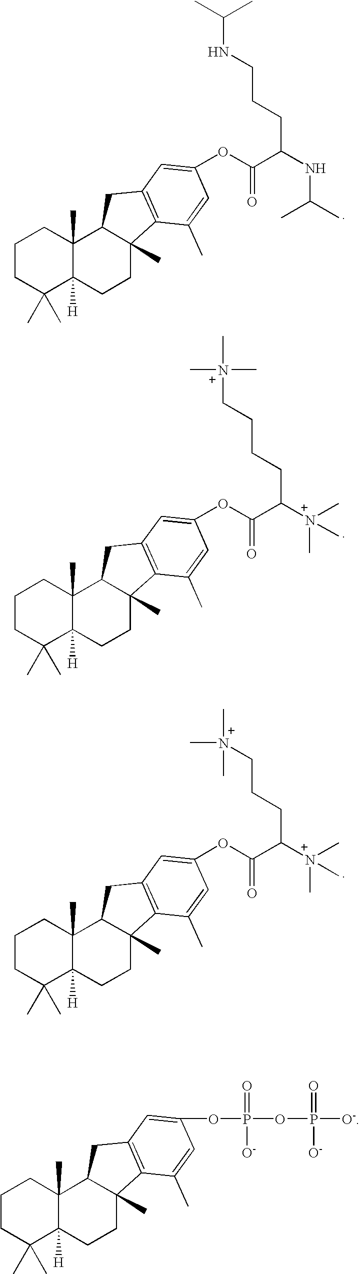 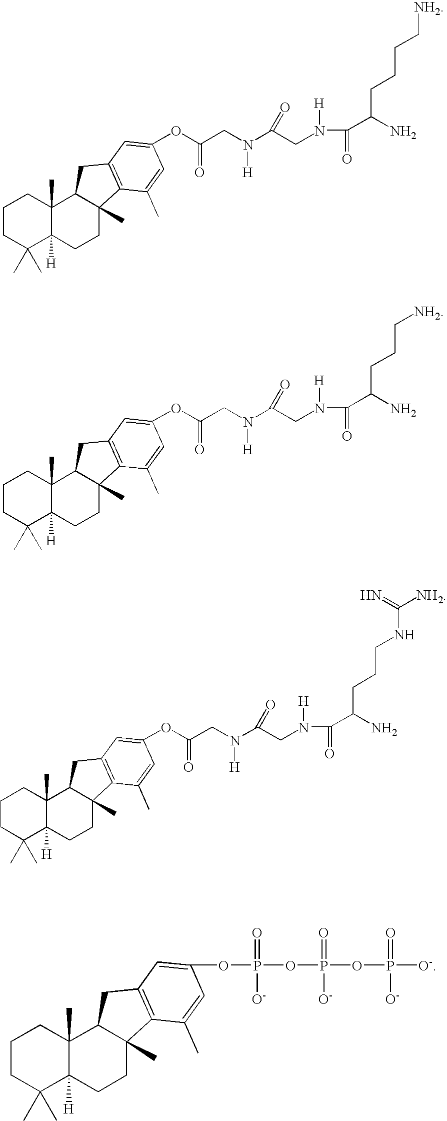
wherein each R is independently H or a C₁ to C₁ alkyl.

113. A pharmaceutical composition comprising a compound of any one of claims 93 to 95 or salt thereof and a pharmaceutically acceptable excipient.

114. A method of treatment or prophylaxis of an inflammatory, neoplastic, hematopoetic or immune disorder or condition, comprising administering to a patient in need of such treatment or prophylaxis an effective amount of the pharmaceutical composition of claim 113.

115. The method of claim 114, wherein the neoplastic disorder or condition is a blood cancer, multiple myeloma, chronic myeloid leukemia or acute myelogenous leukemia.

116. The method of claim 114 wherein the immune disorder or condition is an autoimmune disorder or condition.

117. The method of claim 114 wherein the disorder or condition is an inflammatory disorder or condition.

118. The method of claim 117 wherein the inflammatory disorder or condition is rheumatoid arthritis or inflammatory bowel syndrome.

119. The method of claim 117 wherein the inflammatory disorder or condition is allergy.
